(19) United States
(12) Patent Application Publication
(10) Pub. No.: US 2022/0265834 A1
(43) Pub. Date: Aug. 25, 2022

(54) BCL-2 PROTEIN INHIBITORS

(71) Applicant: Recurium IP Holdings, LLC, San Diego, CA (US)

(72) Inventors: Joseph Robert Pinchman, San Diego, CA (US); Kevin Duane Bunker, Escondido, CA (US); Peter Qinhua Huang, San Diego, CA (US)

(21) Appl. No.: 17/597,474

(22) PCT Filed: Jul. 8, 2020
(86) PCT No.: PCT/US2020/041175

§ 371 Date: Jan. 6, 2022

Related U.S. Application Data
(60) Provisional application No. 62/872,593, filed on Jul. 10, 2019.

(51) Int. Cl. A61K 47/55; A61K 47/54; A61P 35/00

(52) U.S. Cl. CPC A61K 47/552; A61K 47/54; A61P 35/00; A61K 47/545

(57) ABSTRACT

Various Bcl-2 protein inhibitors are described, along with methods of using them to treat conditions characterized by excessive cellular proliferation, such as cancer and tumors. In various embodiments the Bcl-2 protein inhibitors are compounds or pharmaceutically acceptable salts of the following Formula (I), where the variables in Formula (I) are defined herein.

### INCORPORATION BY REFERENCE TO ANY PRIORITY APPLICATIONS

This application claims priority to U.S. Provisional Application Ser. No. 62/872,593, filed Jul. 10, 2019, which is hereby incorporated by reference in its entirety.

### BACKGROUND

### Field

This application relates to compounds that inhibit and/or degrade proteins in the Bcl-2 family to treat conditions characterized by excessive cellular proliferation, such as cancer and tumors.

### Description of the Field

Proteins in the Bcl-2 family contain Bcl-2 homology (BH) domains and regulate apoptosis by modulating mitochondrial outer membrane permeabilization (MOMP). Members of the Bcl-2 family have up to four BH domains, referred to as BH1, BH2, BH3 and BH4. All four domains are conserved in the anti-apoptotic Bcl-2 family members Bcl-2, Bcl-xL, Bcl-W, Mcl-1 and A1/Bfl-1.

A number of compounds that inhibit anti-apoptotic Bcl-2 proteins have been evaluated for their ability to treat lymphomas and other types of cancer. Navitoclax, a dual Bcl-2/xL inhibitor, has been evaluated in Phase I/II clinical trials for the treatment of chronic lymphocytic leukemia (CLL). However, its efficacy in the study population was reduced by dosage limitations due to the occurrence of thrombocytopenia, a side effect of inhibiting Bcl-xL.

Venetoclax is the first Bcl-2 inhibitor to be approved by the FDA. It is available commercially from AbbVie Inc. under the tradename VENCLEXTA. It is currently indicated as a second line treatment for patients with CLL or small lymphocytic lymphoma (SLL).

The FDA approval of Venetoclax represents a milestone in the development of Bcl-2 protein inhibitors. However, there remains a need for improved compounds that inhibit and/or degrade proteins in the Bcl-2 family.

### SUMMARY

Various embodiments provide compounds of the Formula (I) and methods of using them as summarized in the claims below.

### BRIEF DESCRIPTION OF THE DRAWINGS

FIG. 1 illustrates a general synthetic scheme for preparing compounds of the Formula (I).

FIG. 2 illustrates a general multistep synthetic scheme for preparing compounds of the Formula (I).

FIG. 3 illustrates a general multistep synthetic scheme for preparing compounds of the Formula (I).

FIG. 4 illustrates results indicating that the compounds of Examples 2, 4, 5, 6, 9, and 10 induce Bcl-xL degradation in MOLT-4 cells at 100 nM concentrations.

FIG. 5 illustrates results indicating that the compounds of Examples 2, 4, 5, 6, 9, and 10 induce Bcl-xL degradation in MOLT-4 cells at 100 nM concentrations.

FIG. 6 illustrates results indicating that the compounds of Examples 2 and 3 can induce Bcl-xL degradation in MOLM-13 cells in a dose dependent manner.

FIG. 7 illustrates results indicating that Bcl-xL degradation induced by the compound of Examples 2, 3, and 4 can be inhibited by proteasome inhibitor MG132 in MOLM-13 cells.

### DETAILED DESCRIPTION

Bcl-2 is a critical regulator of programmed cell death (apoptosis). Bcl-2 belongs to the B cell lymphoma 2 (BCL-2) family of proteins, which includes both pro-apoptotic proteins (such as Bak, Bax, Bim, Bid, tBid, Bad, Bik, PUMA, Bnip-1, Hrk, Bmf and Noxa) and anti-apoptotic proteins (such as Bcl-2, Bcl-X_{L}, Bcl-W, Mcl-1 and Bcl-2A1). For example, under normal conditions, Bcl-2 inhibits apoptosis in part by preventing activation of Bak and Bax. Activation of the intrinsic apoptosis pathway (e.g., by cellular stress) inhibits Bcl-2, thus activating Bak and Bax. These proteins facilitate mitochondrial outer membrane permeabilization, releasing cytochrome c and Smac. This initiates the caspase signaling pathway, ultimately resulting in cell death. Dysregulation of Bcl-2 leads to sequestration of cell-death-promoting proteins, leading to evasion of apoptosis. This process contributes to malignancy, and facilitates cell survival under other disadvantageous conditions, such as during viral infection. Inhibition of Bcl-2 (e.g., by degrading Bcl-2 protein and/or by inhibiting binding) disrupts sequestration of pro-apoptotic proteins, restoring apoptotic signaling, and promoting damaged cells to undergo programmed cell death. Therefore, inhibition of proteins in the Bcl-2 family (e.g., by inhibition and/or degradation of Bcl-2 protein and/or Bcl-X_{L} protein) has the potential to ameliorate or treat cancers and tumors.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl) and a di-substituted amine(alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

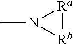

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

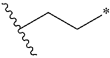 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group (e.g.,

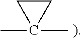

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SW” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The terms “amino” and “unsubstituted amino” as used herein refer to a
NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. A mono-substituted amine group can include, for example, a mono-alkylamine group, a mono-C₁-C₆ alkylamine group, a mono-arylamine group, a mono-C₆-C₁₀ arylamine group and the like. Examples of mono-substituted amine groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. A di-substituted amine group can include, for example, a di-alkylamine group, a di-C₁-C₆ alkylamine group, a di-arylamine group, a di-C₆-C₁₀ arylamine group and the like. Examples of di-substituted amine groups include, but are not limited to, —N(methyl)₂, —N(phenyl)(methyl), —N(ethyl)(methyl) and the like.

As used herein, “mono-substituted amine(alkyl)” group refers to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, a mono-arylamine(alkyl group), a mono-C₆-C₁₀ arylamine(C₁-C₆ alkyl) group and the like. Examples of mono-substituted amine(alkyl) groups include, but are not limited to, —CH₂NH(methyl), —CH₂NH(phenyl), —CH₂CH₂NH(methyl), —CH₂CH₂NH(phenyl) and the like.

As used herein, “di-substituted amine(alkyl)” group refers to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, a di-arylamine(alkyl) group, a di-C₆-C₁₀ arylamine(C₁-C₆ alkyl) group and the like. Examples of di-substituted amine(alkyl) groups include, but are not limited to, —CH₂N(methyl)₂, —CH₂N(phenyl)(methyl), —NCH₂(ethyl)(methyl), —CH₂CH₂N(methyl)₂, —CH₂CH₂N(phenyl)(methyl), —NCH₂CH₂(ethyl)(methyl) and the like.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

The term “Bcl protein inhibition” and similar terms refers to inhibiting the activity or function of a Bcl protein, e.g., by degrading the Bcl protein and/or by inhibiting the binding of an anti-apoptic Bcl protein (such as Bcl-2, Bcl-X_{L}, Bcl-W, Mcl-1 and Bcl-2A1) to a pro-apoptotic Bcl protein (such as Bak, Bax, Bim, Bid, tBid, Bad, Bik, PUMA, Bnip-1, Hrk, Bmf and Noxa). Similarly, the term “Bcl protein inhibitor” refers to an agent (including small molecules and proteins) that inhibit the binding of an anti-apoptic Bcl protein (such as Bcl-2, Bcl-X_{L}, Bcl-W, Mcl-1 and Bcl-2A1) to a pro-apoptotic Bcl protein (such as Bak, Bax, Bim, Bid, tBid, Bad, Bik, PUMA, Bnip-1, Hrk, Bmf and Noxa). In addition to its binding inhibition function, a Bcl protein inhibitor may also have the function of degrading the Bcl protein. Such a Bcl protein inhibitor may be referred to herein as a Bcl protein degrader, particularly when degradation is the predominant mechanism of Bcl protein inhibition. See, e.g., WO 2019144117 (disclosing Bcl protein degraders that are bivalent compounds that connect a Bcl-2 small molecule inhibitor or ligand to an E3 ligase binding moiety). Bcl protein inhibitors include, but are not limited to venetoclax, navitoclax, obatoclax, S55746, APG-2575, ABT-737, AMG176, AZD5991 and APG-1252. Additional Bcl protein inhibitors include, but are not limited to, compounds disclosed in PCT Application Publication Nos. WO2017/132474, WO 2014/113413 and WO 2013/110890, U.S. Patent Application Publication No. 2015/0051189 and Chinese Patent Application No. CN 106565607, which are each incorporated herein by reference for the limited purpose of disclosing additional Bcl protein inhibitors. As will be understood by those of skill in the art, there are numerous methods of evaluating protein binding interactions, including, but not limited to co-immunoprecipitation, fluorescence resonance energy transfer (FRET), surface plasmon resonance (SPR) and fluorescence polarization/anisotropy.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to a compound of Formula (I), or a pharmaceutically acceptable salt thereof, having the structure:

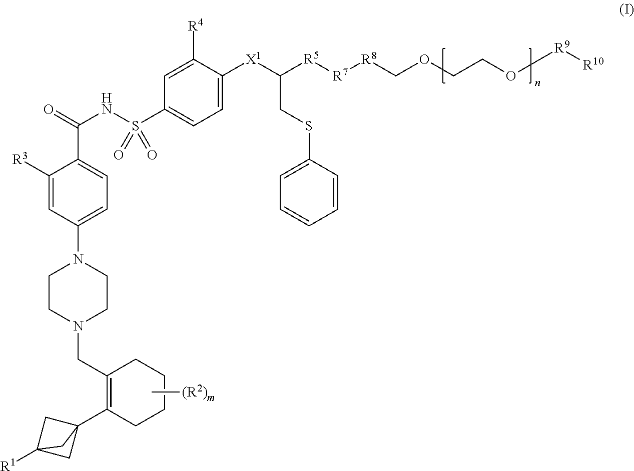

In various embodiments, the variables in Formula (I) are defined as follows:

R¹ can be selected from hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine.

Each R² can be independently selected from a halogen, a substituted or unsubstituted C₁-C₆ alkyl, a substituted or unsubstituted C₁-C₆ haloalkyl and a substituted or unsubstituted C₃-C₆ cycloalkyl; or when m is 2 or 3, each R² can be independently selected from a halogen, a substituted or unsubstituted C₁-C₆ alkyl, a substituted or unsubstituted C₁-C₆ haloalkyl and a substituted or unsubstituted C₃-C₆ cycloalkyl, or two R² groups taken together with the atom(s) to which they are attached can form a substituted or unsubstituted C₃-C₆ cycloalkyl or a substituted or unsubstituted 3 to 6 membered heterocyclyl.

R³ can be hydrogen or halogen.

R⁴ can be selected from NO₂, S(O)R⁶, SO₂R⁶, halogen, cyano and an unsubstituted C₁-C₆ haloalkyl.

R⁵ can be a substituted or unsubstituted C₁-C₆ alkylene, a substituted or unsubstituted —(C₁-C₆ alkylene)-Het-, a substituted or unsubstituted —(C₁-C₆ alkylene)-O—, a substituted or unsubstituted —(C₁-C₆ alkylene)-NH—, a substituted or unsubstituted —(C₁-C₆ alkylene)-Het-O—, a substituted or unsubstituted —(C₁-C₆ alkylene)-Het-NH—, a substituted or unsubstituted —(C₁-C₆ alkylene)-N(C₁-C₆ alkyl)-, a substituted or unsubstituted —(C₁-C₆ alkylene)-Het-N(C₁-C₆ alkyl)-, a substituted or unsubstituted —(C₁-C₆ alkylene)-(C═O)—O— or a substituted or unsubstituted —(C₁-C₆ alkylene)-Het-(C═O)—O—, where Het is a substituted or unsubstituted 3 to 10 membered heterocyclyl.

R⁶ can be a substituted or unsubstituted C₁-C₆ alkyl, a substituted or unsubstituted C₁-C₆ haloalkyl or a substituted or unsubstituted C₃-C₆ cycloalkyl.

R⁷ can be absent, a substituted or unsubstituted C₁-C₆ alkylene, —(C═O)—, —(C═S)—, —(C═O)—NH—, —(C═O)—O—, —(C═S)—NH—, a substituted or unsubstituted (C₁-C₆ alkylene)-O—, or a substituted or unsubstituted (C₁-C₆ alkylene)-NH—.

R⁸ can be absent, a substituted or unsubstituted C₁-C₆ alkylene, a substituted or unsubstituted —(C₁-C₆ alkylene)-(C₆-C₁₂ aryl)-, a substituted or unsubstituted —(C₁-C₆ alkylene)-(C₃-C₁₀ cycloalkyl)-, a substituted or unsubstituted —(C₁-C₆ alkylene)-(C₃-C₁₀ heterocyclyl)-, or a substituted or unsubstituted —(C₁-C₆ alkylene)-(5 to 10 membered heteroaryl)-.

X¹ can be —O— or —NH—; m can be 0, 1, 2 or 3; and n can be 0, 1, 2, 3, 4 or 5.

R⁹ can be a substituted or unsubstituted C₁-C₁₀ alkylene, a substituted or unsubstituted —(C₁-C₆ alkylene)-O—, a substituted or unsubstituted —(C₁-C₆ alkylene)-NH—, a substituted or unsubstituted —(C₁-C₆ alkylene)-NH—(C₁-C₆ alkylene)-, a substituted or unsubstituted —(C₁-C₆ alkylene)-(C═O)NH—, a substituted or unsubstituted —(C₁-C₆ alkylene)-NH—(C₁-C₆ alkylene)-NH—, a substituted or unsubstituted —(C₁-C₆ alkylene)-NH—(C₁-C₆ alkylene)-O—, a substituted or unsubstituted —(C₁-C₆ alkylene)-NH(C═O)—(C₁-C₆ alkylene)-NH—, a substituted or unsubstituted —(C₁-C₆ alkylene)-NH(C═O)—(C₁-C₆ alkylene)-O—, a substituted or unsubstituted —(C₁-C₆ alkylene)-NH(C═O)—(C₁-C₆ alkylene)-, a substituted or unsubstituted —(C₁-C₆ alkylene)-NH—(C₁-C₆ alkylene)-NH(C═O)—(C₁-C₆ alkylene)-NH—, a substituted or unsubstituted —(C₁-C₆ alkylene)-NH—(C₁-C₆ alkylene)-NH(C═O)—(C₁-C₆ alkylene)-O—, a substituted or unsubstituted —(C₁-C₆ alkylene)-NH—(C₁-C₆ alkylene)-NH(C═O)—(C₁-C₆ alkylene)-, a substituted or unsubstituted —(C₁-C₆ alkylene)-(C═O)NH—(C₁-C₆ alkylene)-, a substituted or unsubstituted —(C₁-C₆ alkylene)-(C═O)NH—(C₁-C₆ alkylene)-NH—, a substituted or unsubstituted —(C₁-C₆ alkylene)-(C═O)NH—(C₁-C₆ alkylene)-O—, a substituted or unsubstituted —(C₁-C₆ alkylene)-NH(C═O)—(C₁-C₆ alkylene)-(C═O)NH—, a substituted or unsubstituted —(C₁-C₆ alkylene)-NH—(C₁-C₆ alkylene)-(C═O)NH—, or a substituted or unsubstituted —(C₁-C₆ alkylene)-NH—(C₁-C₆ alkylene)-(C═O)NH—(C₁-C₆ alkylene)-.

R¹⁰ can be selected from the following:

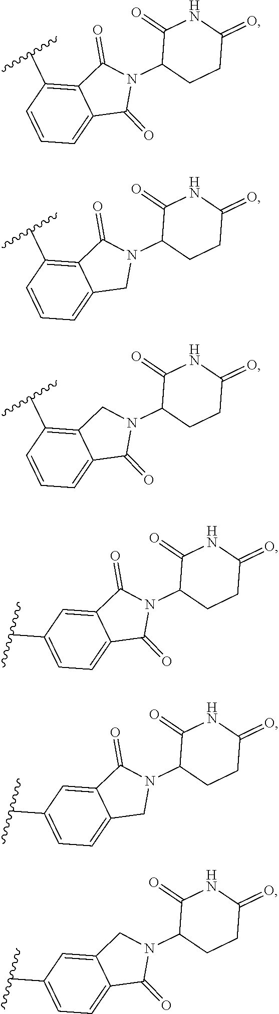 [SMILES: *c1cccc2c1C(=O)N(C1CCC(=O)NC1=O)C2=O ; *c1cccc2c1C(=O)N(C1CCC(=O)NC1=O)C2 ; *c1cccc2c1CN(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O]

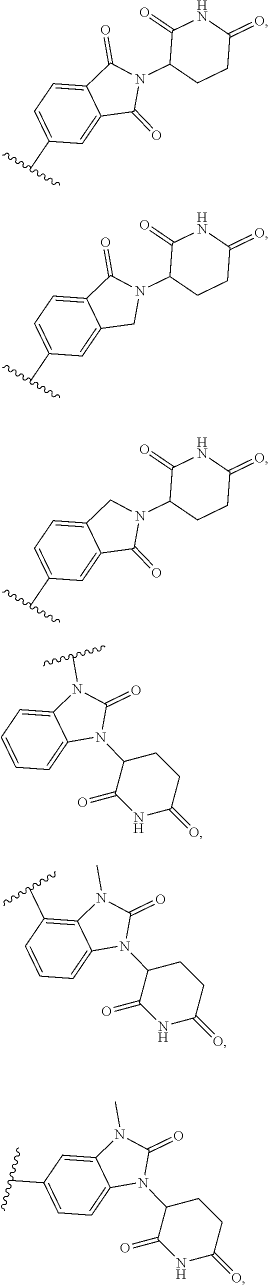 [SMILES: *c1ccc2c(c1)C(=O)N(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *n1c(=O)n(C2CCC(=O)NC2=O)c2ccccc21 ; *c1cccc2c1n(C)c(=O)n2C1CCC(=O)NC1=O ; *c1ccc2c(c1)n(C)c(=O)n2C1CCC(=O)NC1=O]

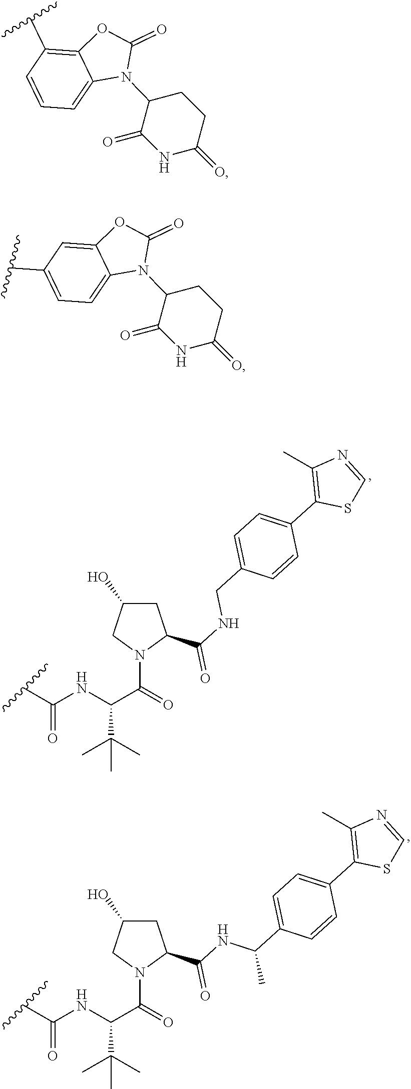 [SMILES: *c1cccc2c1oc(=O)n2C1CCC(=O)NC1=O ; *c1ccc2c(c1)oc(=O)n2C1CCC(=O)NC1=O ; *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

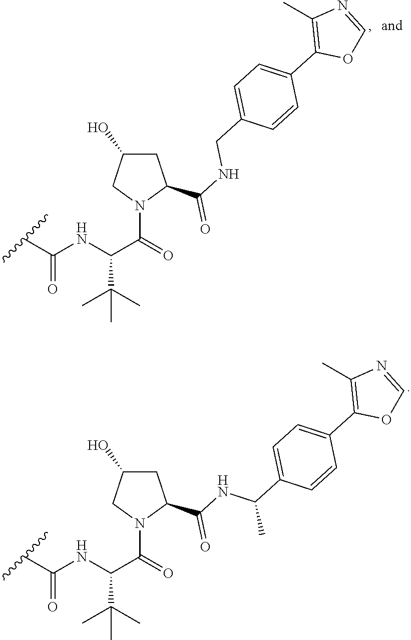 [SMILES: *C(=O)N[C@H](C(=O)N1C[C@H](O)C[C@H]1C(=O)NCc1ccc(-c2ocnc2C)cc1)C(C)(C)C ; *C(=O)N[C@H](C(=O)N1C[C@H](O)C[C@H]1C(=O)N[C@@H](C)c1ccc(-c2ocnc2C)cc1)C(C)(C)C]

In some embodiments, R¹ can be halogen, for example, fluoro, chloro, bromo or iodo. In some embodiments, R¹ can be fluoro. In some embodiments, R¹ can be chloro. In some embodiments, R¹ can be hydrogen.

In some embodiments, R¹ can be a substituted or unsubstituted C₁-C₆ alkyl. For example, in some embodiments, R¹ can be a substituted C₁-C₆ alkyl. In other embodiments, R¹ can be an unsubstituted C₁-C₆ alkyl. Examples of suitable C₁-C₆ alkyl groups include, but are not limited to methyl, ethyl, n-propyl, isopropyl, n-butyl, isobutyl, tert-butyl, pentyl (branched and straight-chained) and hexyl (branched and straight-chained). In some embodiments, R¹ can be an unsubstituted methyl or an unsubstituted ethyl.

In some embodiments, R¹ can be a substituted or unsubstituted C₁-C₆ haloalkyl, for example, a substituted or unsubstituted mono-halo C₁-C₆ alkyl, a substituted or unsubstituted di-halo C₁-C₆ alkyl, a substituted or unsubstituted tri-halo C₁-C₆ alkyl, a substituted or unsubstituted tetra-halo C₁-C₆ alkyl or a substituted or unsubstituted penta-halo C₁-C₆ alkyl. In some embodiments, R¹ can be an unsubstituted —CHF₂, —CF₃, —CH₂CF₃, —CF₂CF₃, or —CF₂CH₃. In some embodiments, R¹ is —CH₂F, —CHF₂ or —CF_{3.}

In some embodiments, R¹ can be a substituted or unsubstituted monocyclic or bicyclic C₃-C₆ cycloalkyl. For example, in some embodiments, R¹ can be a substituted monocyclic C₃-C₆ cycloalkyl. In other embodiments, R¹ can be an unsubstituted monocyclic C₃-C₆ cycloalkyl. Examples of suitable monocyclic or bicyclic C₃-C₆ cycloalkyl groups include, but are not limited to cyclopropyl, cyclobutyl, cyclopentyl, [1.1.1]bicyclopentyl and cyclohexyl.

In some embodiments, R¹ can be a substituted or unsubstituted C₁-C₆ alkoxy. For example, in some embodiments, R¹ can be a substituted C₁-C₆ alkoxy. In other embodiments, R¹ can be an unsubstituted C₁-C₆ alkoxy. Examples of suitable C₁-C₆ alkoxy groups include, but are not limited to methoxy, ethoxy, n-propoxy, isopropoxy, n-butoxy, isobutoxy, tert-butoxy, pentoxy (branched and straight-chained) and hexoxy (branched and straight-chained). In some embodiments, R¹ can be an unsubstituted methoxy or an unsubstituted ethoxy.

In some embodiments, R¹ can be an unsubstituted mono-C₁-C₆ alkylamine, for example, methylamine, ethylamine, n-propylamine, isopropylamine, n-butylamine, isobutylamine, tert-butylamine, pentylamine (branched and straight-chained) and hexylamine (branched and straight-chained). In some embodiments, R¹ can be methylamine or ethylamine.

In some embodiments, R¹ can be an unsubstituted di-C₁-C₆ alkylamine. In some embodiments, each C₁-C₆ alkyl in the di-C₁-C₆ alkylamine is the same. In other embodiments, each C₁-C₆ alkyl in the di-C₁-C₆ alkylamine is different. Examples of suitable di-C₁-C₆ alkylamine groups include, but are not limited to di-methylamine, di-ethylamine, (methyl)(ethyl)amine, (methyl)(isopropyl)amine and (ethyl)(isopropyl)amine.

In some embodiments, m can be 0. When m is 0, those skilled in the art understand that the ring to which R² is attached is unsubstituted. In some embodiments, m can be 1. In some embodiments, m can be 2. In some embodiments, m can be 3.

In some embodiments, one R² can be an unsubstituted C₁-C₆ alkyl (for example, methyl, ethyl, n-propyl, isopropyl, n-butyl, isobutyl, tert-butyl, pentyl (branched and straight-chained) and hexyl (branched and straight-chained) and any other R², if present, can be independently selected from halogen (for example, fluoro or chloro), a substituted or unsubstituted C₁-C₆ alkyl (such as those described herein), a substituted or unsubstituted C₁-C₆ haloalkyl (such as those described herein) and a substituted or unsubstituted monocyclic or bicyclic C₃-C₆ cycloalkyl (such as those described herein). In some embodiments, each R² can be independently selected from an unsubstituted C₁-C₆ alkyl, such as those described herein.

In some embodiments, m can be 2; and each R² can be geminal. In some embodiments, m can be 2; and each R² can be vicinal. In some embodiments, m can be 2; and each R² can be an unsubstituted methyl. In some embodiments, m can be 2; and each R² can be a geminal unsubstituted methyl.

In some embodiments, two R² groups can be taken together with the atom(s) to which they are attached to form a substituted or unsubstituted monocyclic C₃-C₆ cycloalkyl. For example, in some embodiments, two R² groups can be taken together with the atom(s) to which they are attached to form a substituted monocyclic C₃-C₆ cycloalkyl, such as those described herein. In other embodiments, two R² groups can be taken together with the atom(s) to which they are attached to form an unsubstituted monocyclic C₃-C₆ cycloalkyl, such as those described herein. In some embodiments, two R² groups can be taken together with the atom to which they are attached to form an unsubstituted cyclopropyl. In some embodiments, two R² groups can be taken together with the atom to which they are attached to form an unsubstituted cyclobutyl.

In some embodiments, two R² groups can be taken together with the atom(s) to which they are attached to form a substituted or unsubstituted monocyclic 3 to 6 membered heterocyclyl. For example, in some embodiments, two R² groups can be taken together with the atom(s) to which they are attached to form a substituted monocyclic 3 to 6 membered heterocyclyl. In other embodiments, two R² groups can be taken together with the atom(s) to which they are attached to form an unsubstituted monocyclic 3 to 6 membered monocyclic heterocyclyl. In some embodiments, the substituted monocyclic 3 to 6 membered heterocyclyl can be substituted on one or more nitrogen atoms. Examples of suitable substituted or unsubstituted monocyclic 3 to 6 membered heterocyclyl groups include, but are not limited to azidirine, oxirane, azetidine, oxetane, pyrrolidine, tetrahydrofuran, imidazoline, pyrazolidine, piperidine, tetrahydropyran, piperazine, morpholine, thiomorpholine and dioxane.

In some embodiments, R³ can be hydrogen. In some embodiments, R³ can be halogen. In some embodiments, R³ can be fluoro or chloro.

In some embodiments, R⁴ can be NO₂. In some embodiments, R⁴ can be cyano. In some embodiments, R⁴ can be halogen.

In some embodiments, R⁴ can be an unsubstituted C₁-C₆ haloalkyl, such as those described herein. In some embodiments, R⁴ can be —CF₃.

In some embodiments, R⁴ can be S(O)R⁶. In some embodiments, R⁴ can be SO₂R⁶. In some embodiments, R⁴ can be SO₂CF₃.

In some embodiments, R⁶ can be a substituted or unsubstituted C₁-C₆ alkyl. For example, in some embodiments, R⁶ can be a substituted C₁-C₆ alkyl, such as those described herein. In other embodiments, R⁶ can be an unsubstituted C₁-C₆ alkyl, such as those described herein.

In some embodiments, R⁶ can be a substituted or unsubstituted monocyclic or bicyclic C₃-C₆ cycloalkyl. For example, in some embodiments, R⁶ can be a substituted monocyclic or bicyclic C₃-C₆ cycloalkyl. In other embodiments, R⁶ can be an unsubstituted monocyclic or bicyclic C₃-C₆ cycloalkyl. Examples of suitable monocyclic or bicyclic C₃-C₆ cycloalkyl groups include, but are not limited to cyclopropyl, cyclobutyl, cyclopentyl, [1.1.1]bicyclopentyl and cyclohexyl.

In some embodiments, R⁶ can be a substituted or unsubstituted C₁-C₆ haloalkyl, such as those described herein. In some embodiments, R⁶ can be —CF₃.

In some embodiments, R⁵ can be a substituted or unsubstituted C₁-C₆ alkylene. For example, in some embodiments R⁵ can be a —(CH₂)ₚ₁— group, where p1 is 1, 2, 3, 4, 5 or 6. In some embodiments, R⁵ can be a substituted or unsubstituted —(C₁-C₆ alkylene)-Het-, where Het is a substituted or unsubstituted 3 to 10 membered heterocyclyl. For example, in some embodiments R⁵ can be a —(CH₂)ₚ-Het group, where p is 1, 2, 3, 4, 5 or 6. Examples of suitable Het groups include 4 to 6 membered heterocyclyl groups such as azetidinyl, pyrrolidinyl, piperidinyl, or piperazinyl. In some embodiments, R⁵ can be a substituted or unsubstituted —(C₁-C₆ alkylene)-O— or a substituted or unsubstituted C₆ alkylene)-Het-O—. For example, in some embodiments R⁵ can be a —(CH₂)ₚ₁—O— group or a —(CH₂)ₚ₁-Het-O— group, where p1 is 1, 2, 3, 4, 5 or 6. In some embodiments, R⁵ can be a substituted or unsubstituted —(C₁-C₆ alkylene)-NH— or a substituted or unsubstituted —(C₁-C₆ alkylene)-Het-NH—. For example, in some embodiments R⁵ can be a —(CH₂)ₚ₁—NH— group or a —(CH₂)ₚ₁-Het-NH— group, where p1 is 1, 2, 3, 4, 5 or 6. In some embodiments, R⁵ can be a substituted or unsubstituted —(C₁-C₆ alkylene)-N(C₁-C₆ alkyl)- or a substituted or unsubstituted (C₁-C₆ alkylene)-Het-N(C₁-C₆ alkyl)-. For example, in some embodiments R⁵ can be a —(CH₂)ₚ₁—N(C₁-C₆ alkyl)- group or a —(CH₂)ₚ₁-Het-N(C₁-C₆ alkyl)- group, where p1 is 1, 2, 3, 4, 5 or 6. In some embodiments, R⁵ can be a substituted or unsubstituted —(C₁-C₆ alkylene)-(C═O)—O— or a substituted or unsubstituted —(C₁-C₆ alkylene)-Het-(C═O)—O—. For example, in some embodiments R⁵ can be a —(CH₂)ₚ₁—(C═O)—O— or —(CH₂)ₚ₁-Het-(C═O)—O—group, where p1 is 1, 2, 3, 4, 5 or 6.

In some embodiments, R⁷ can be absent, in which case R⁵ can be joined directly to R⁸, or if R⁸ is absent, directly to the next atom adjoining R⁸. In other embodiments, R⁷ can be a substituted or unsubstituted C₁-C₆ alkylene. For example, in some embodiments R⁷ can be a —(CH₂)ₚ₁— group, where p1 is 1, 2, 3, 4, 5 or 6. In other embodiments, R⁷ can be —(C═O)—, —(C═S)—, —(C═O)—NH—, —(C═O)—O—, or —(C═S)—NH—. In other embodiments, R⁷ can be a substituted or unsubstituted —(C₁-C₆ alkylene)-O— or a substituted or unsubstituted —(C₁-C₆ alkylene)-NH—. For example, R⁷ can be —(CH₂)ₚ₁—O— or —(CH₂)ₚ₁—NH—, where p1 is 1, 2, 3, 4, 5 or 6.

In various embodiments, R⁵ and R⁷ are selected together such that —R⁵-R⁷— is selected from:

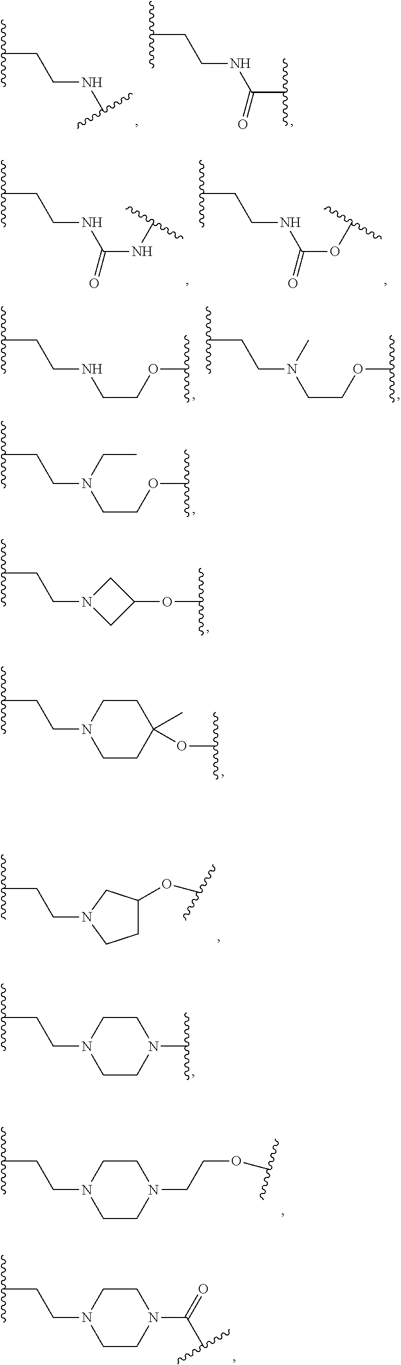

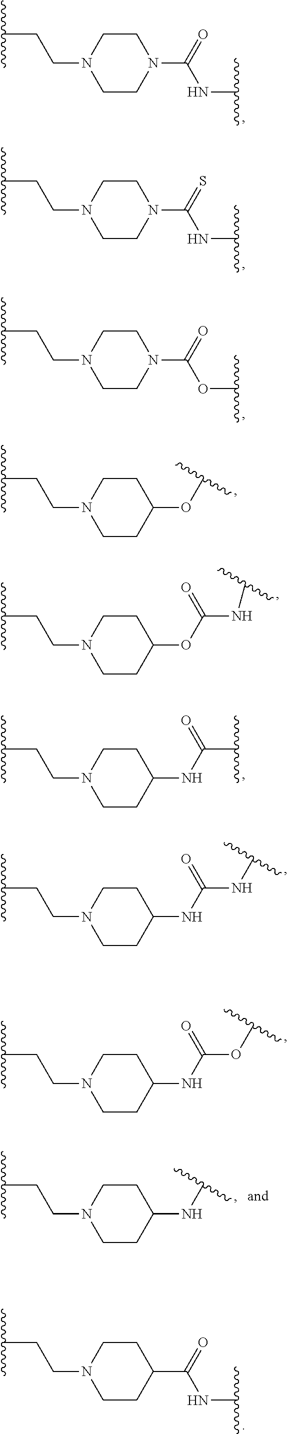

For example, in some embodiments, R⁵ and R⁷ are selected together such that —R⁵-R⁷— is selected from:

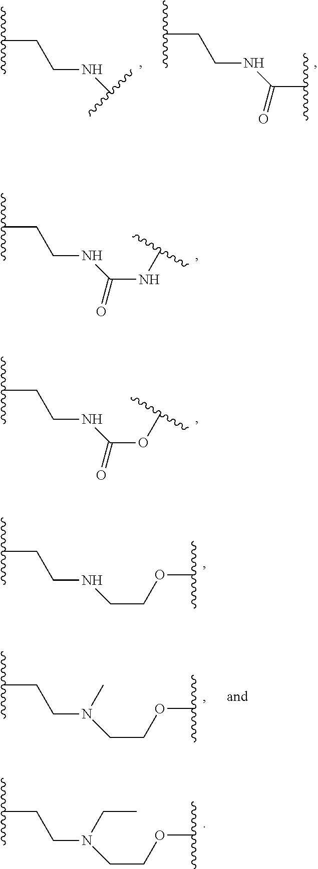

In other embodiments, R⁵ and R⁷ are selected together such that —R⁵-R⁷— is selected from:

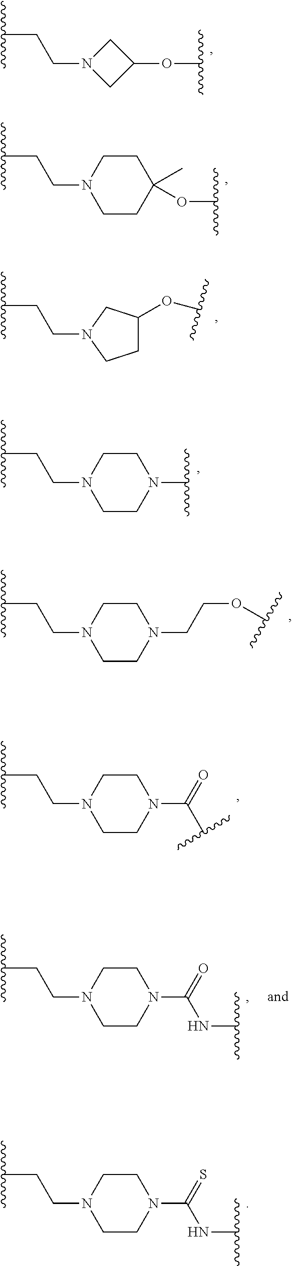

In other embodiments, R⁵ and R⁷ are selected together such that —R⁵-R⁷— is selected from:

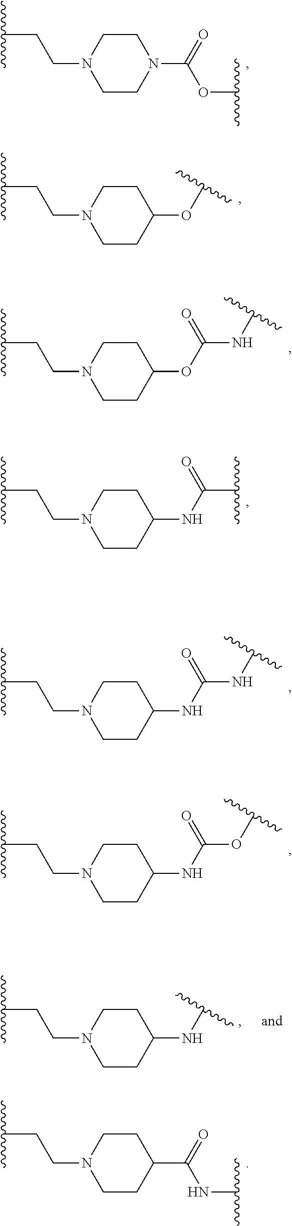

In some embodiments, R⁸ can be absent, in which case R⁷ (if present; if not, then R⁵) can be joined directly to the next atom adjoining R⁸. In other embodiments, R⁸ can be a substituted or unsubstituted C₁-C₆ alkylene. For example, in some embodiments R⁸ can be a —(CH₂)ₚ₁— group, where p1 is 1, 2, 3, 4, 5 or 6. In other embodiments, R⁸ can be a substituted or unsubstituted —(C₁-C₆ alkylene)-(C₆-C₁₂ aryl)-, a substituted or unsubstituted —(C₁-C₆ alkylene)-(C₃-C₁₀ cycloalkyl)-, a substituted or unsubstituted —(C₁-C₆ alkylene)-(C₃-C₁₀ heterocyclyl)-, or a substituted or unsubstituted —(C₁-C₆ alkylene)-(5 to 10 membered heteroaryl)-. For example, R⁸ can be a substituted or unsubstituted —(CH₂)ₚ₁—(C₆-C₁₂ aryl)-, a substituted or unsubstituted —(CH₂)ₚ₁—(C₃-C₁₀ cycloalkyl)-, a substituted or unsubstituted —(CH₂)ₚ₁—(C₃-C₁₀ heterocyclyl)-, or a substituted or unsubstituted —(CH₂)ₚ₁-(5 to 10 membered heteroaryl)-, where p1 is 1, 2, 3, 4, 5 or 6.

In various embodiments, X¹ can be —O—. In other embodiments, X¹ can be —NH—.

In some embodiments, n is zero, in which case the ethyleneoxy group of the formula —(CH₂CH₂O)ₙ— in Formula (I) is absent and the R⁹ group is joined directly to the oxygen atom adjoining the ethyleneoxy group. In other embodiments, n is 1, 2, 3, 4, or 5, in which case the ethyleneoxy group of the formula —(CH₂CH₂O)ₙ— in Formula (I) is present.

In various embodiments, R⁹ can be a substituted or unsubstituted C₁-C₁₀ alkylene, a substituted or unsubstituted —(C₁-C₆ alkylene)-O—, a substituted or unsubstituted —(C₁-C₆ alkylene)-NH—, a substituted or unsubstituted —(C₁-C₆ alkylene)-NH—(C₁-C₆ alkylene)-, a substituted or unsubstituted —(C₁-C₆ alkylene)-(C═O)NH—, a substituted or unsubstituted —(C₁-C₆ alkylene)-NH—(C₁-C₆ alkylene)-NH—, a substituted or unsubstituted —(C₁-C₆ alkylene)-NH—(C₁-C₆ alkylene)-O—, a substituted or unsubstituted —(C₁-C₆ alkylene)-NH(C═O)—(C₁-C₆ alkylene)-NH—, a substituted or unsubstituted —(C₁-C₆ alkylene)-NH(C═O)—(C₁-C₆ alkylene)-O—, a substituted or unsubstituted —(C₁-C₆ alkylene)-NH(C═O)—(C₁-C₆ alkylene)-, a substituted or unsubstituted —(C₁-C₆ alkylene)-NH—(C₁-C₆ alkylene)-NH(C═O)—(C₁-C₆ alkylene)-NH—, a substituted or unsubstituted —(C₁-C₆ alkylene)-NH—(C₁-C₆ alkylene)-NH(C═O)—(C₁-C₆ alkylene)-O—, a substituted or unsubstituted —(C₁-C₆ alkylene)-NH—(C₁-C₆ alkylene)-NH(C═O)—(C₁-C₆ alkylene)-, a substituted or unsubstituted —(C₁-C₆ alkylene)-(C═O)NH—(C₁-C₆ alkylene)-, a substituted or unsubstituted —(C₁-C₆ alkylene)-(C═O)NH—(C₁-C₆ alkylene)-NH—, or a substituted or unsubstituted —(C₁-C₆ alkylene)-(C═O)NH—(C₁-C₆ alkylene)-O—, a substituted or unsubstituted —(C₁-C₆ alkylene)-NH(C═O)—(C₁-C₆ alkylene)-(C═O)NH—, a substituted or unsubstituted —(C₁-C₆ alkylene)-NH—(C₁-C₆ alkylene)-(C═O)NH—, or a substituted or unsubstituted —(C₁-C₆ alkylene)-NH—(C₁-C₆ alkylene)-(C═O)NH—(C₁-C₆ alkylene)-.

For example, in various embodiments R⁹ can be a substituted or unsubstituted C₁-C₁₀ alkylene, a substituted or unsubstituted —(C₁-C₆ alkylene)-O—, a substituted or unsubstituted —(C₁-C₆ alkylene)-NH—, a substituted or unsubstituted —(C₁-C₆ alkylene)-NH—(C₁-C₆ alkylene)-, or a substituted or unsubstituted —(C₁-C₆ alkylene)-(C═O)NH—. In other embodiments R⁹ can be a substituted or unsubstituted —(C₁-C₆ alkylene)-NH—(C₁-C₆ alkylene)-NH—, a substituted or unsubstituted —(C₁-C₆ alkylene)-NH—(C₁-C₆ alkylene)-O—, a substituted or unsubstituted —(C₁-C₆ alkylene)-NH(C═O)—(C₁-C₆ alkylene)-NH—, a substituted or unsubstituted —(C₁-C₆ alkylene)-NH(C═O)—(C₁-C₆ alkylene)-O—, or a substituted or unsubstituted —(C₁-C₆ alkylene)-NH(C═O)—(C₁-C₆ alkylene)-.

In other embodiments R⁹ can be a substituted or unsubstituted —(C₁-C₆ alkylene)-NH—(C₁-C₆ alkylene)-NH(C═O)—(C₁-C₆ alkylene)-NH—, a substituted or unsubstituted —(C₁-C₆ alkylene)-NH—(C₁-C₆ alkylene)-NH(C═O)—(C₁-C₆ alkylene)-O—, a substituted or unsubstituted —(C₁-C₆ alkylene)-NH—(C₁-C₆ alkylene)-NH(C═O)—(C₁-C₆ alkylene)-, a substituted or unsubstituted —(C₁-C₆ alkylene)-(C═O)NH—(C₁-C₆ alkylene)-, a substituted or unsubstituted —(C₁-C₆ alkylene)-(C═O)NH—(C₁-C₆ alkylene)-NH—, a substituted or unsubstituted —(C₁-C₆ alkylene)-(C═O)NH—(C₁-C₆ alkylene)-O—, a substituted or unsubstituted —(C₁-C₆ alkylene)-NH(C═O)—(C₁-C₆ alkylene)-(C═O)NH—, a substituted or unsubstituted —(C₁-C₆ alkylene)-NH—(C₁-C₆ alkylene)-(C═O)NH—, or a substituted or unsubstituted —(C₁-C₆ alkylene)-NH—(C₁-C₆ alkylene)-(C═O)NH—(C₁-C₆ alkylene)-.

In various embodiments variables are described herein, such as R⁹, that contain a C₁-C₆ alkylene group or a group containing one or more C₁-C₆ alkylene groups. Such C₁-C₆ alkylene groups as described herein can be a —(CH₂)ₚ₁— group, where p1 is 1, 2, 3, 4, 5 or 6.

In various embodiments, R¹⁰ can be a group selected from

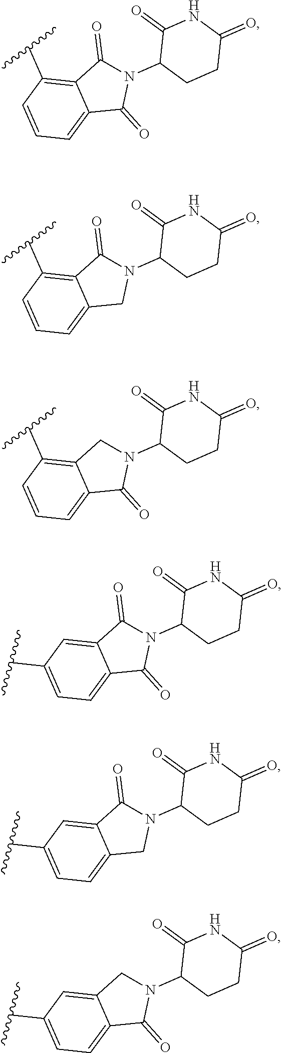 [SMILES: *c1cccc2c1C(=O)N(C1CCC(=O)NC1=O)C2=O ; *c1cccc2c1C(=O)N(C1CCC(=O)NC1=O)C2 ; *c1cccc2c1CN(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O]

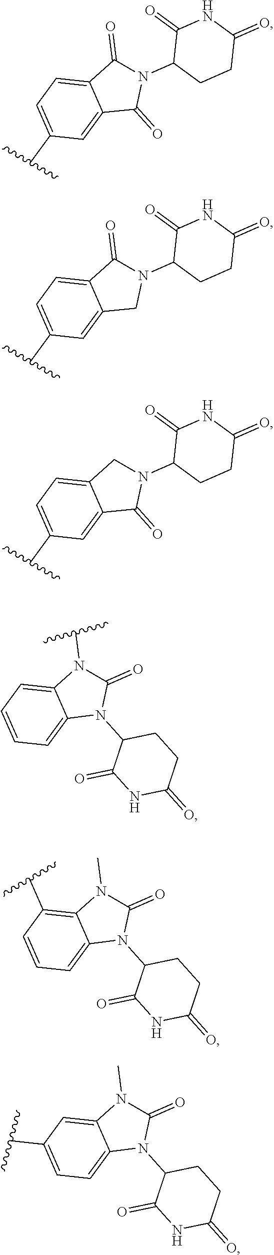 [SMILES: *c1ccc2c(c1)C(=O)N(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *n1c(=O)n(C2CCC(=O)NC2=O)c2ccccc21 ; *c1cccc2c1n(C)c(=O)n2C1CCC(=O)NC1=O ; *c1ccc2c(c1)n(C)c(=O)n2C1CCC(=O)NC1=O]

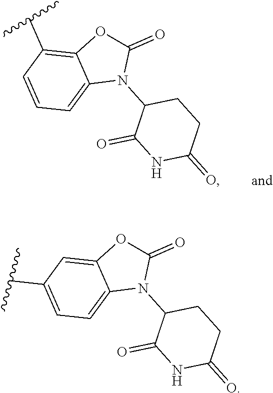 [SMILES: *c1cccc2c1oc(=O)n2C1CCC(=O)NC1=O ; "and" ; *c1ccc2c(c1)oc(=O)n2C1CCC(=O)NC1=O]

In other embodiments, R¹⁰ can be a group selected from

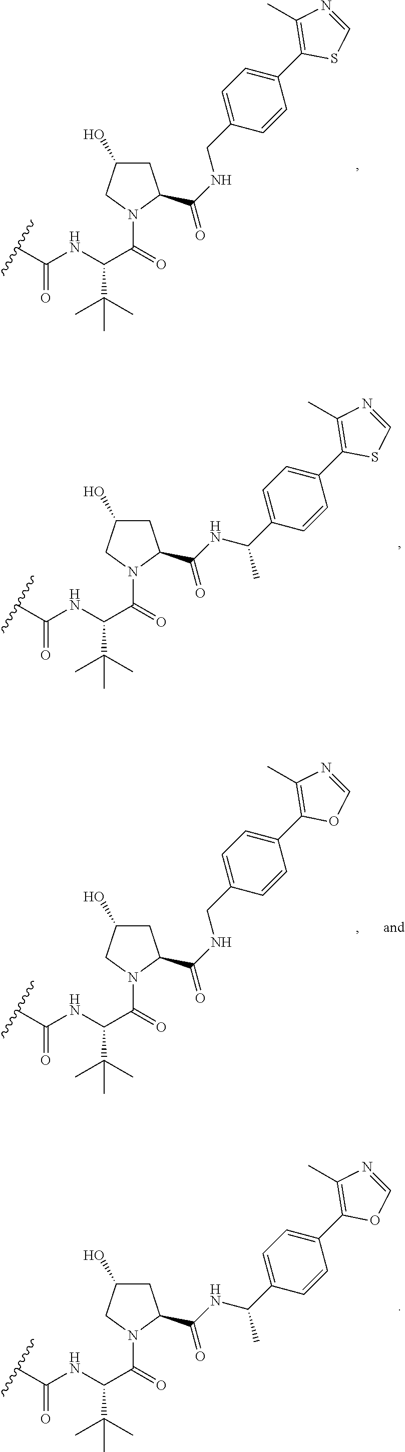 [SMILES: *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)NCc1ccc(-c2ocnc2C)cc1)C(C)(C)C ; *C(=O)N[C@H](C(=O)N1C[C@H](O)C[C@H]1C(=O)N[C@@H](C)c1ccc(-c2ocnc2C)cc1)C(C)(C)C]

In various embodiments, compounds of the Formula (I) are selected from those described in the claims below.

### Synthesis

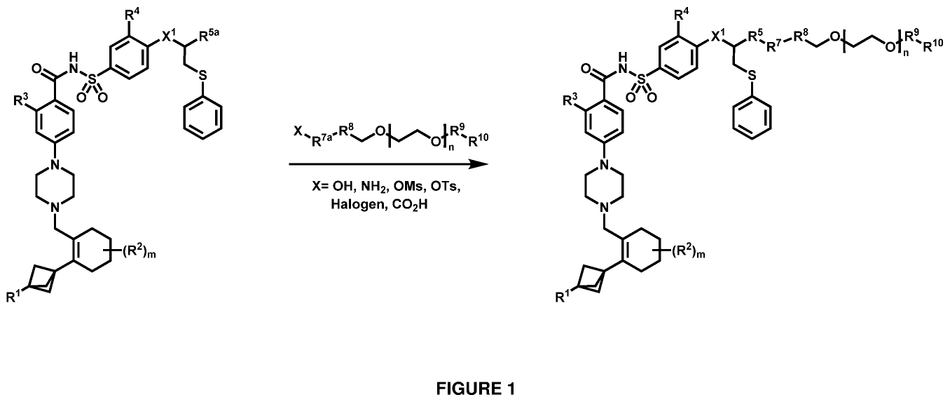

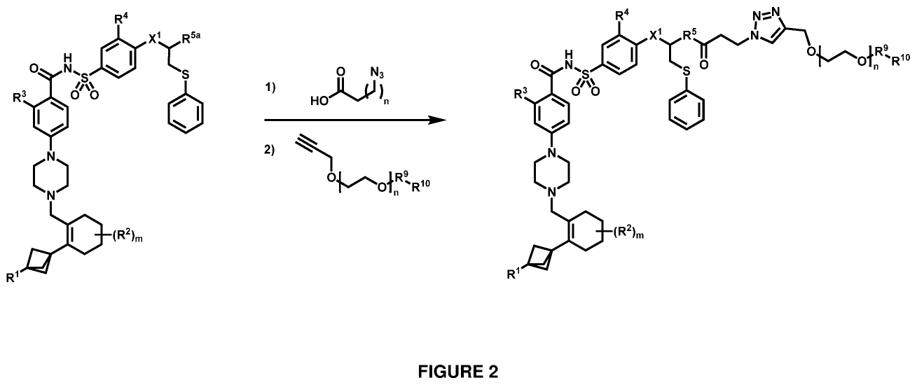

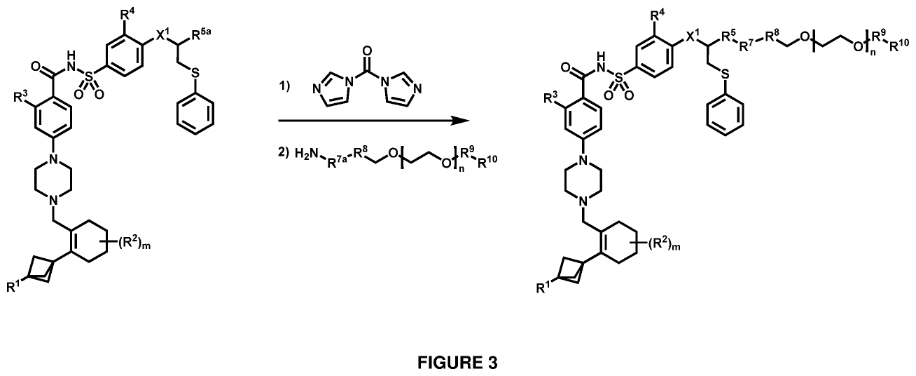

Compounds of the Formula (I), or pharmaceutically acceptable salts thereof, can be made in various ways by those skilled using known techniques as guided by the detailed teachings provided herein, including the Examples provided below. For example, in an embodiment, compounds of the Formula (I) are prepared in accordance with the general scheme illustrated in FIG. 1. For example, compounds of the Formula (I) can be prepared in multiple steps as illustrated in FIGS. 2 and 3. In various embodiments, intermediate compounds useful for making compounds of the Formula (I), or pharmaceutically acceptable salts thereof, can be made as described in PCT Publication Nos. WO 2019/139899, WO 2019/139900, WO 2019/139902, and WO 2019/139907, each of which is hereby incorporated herein by reference and particularly for the purpose of describing intermediate compounds useful for making compounds of the Formula (I), pharmaceutically acceptable salts thereof, and methods of making them. Any preliminary reaction steps required to form starting compounds or other precursors, can be carried out by those skilled in the art. In FIGS. 1-3, the variables R¹, R², R³, R⁴, R⁵, R⁶, R⁷, R⁸, R⁹, R¹⁰, X¹, m and n can be as described elsewhere herein, taking into consideration the synthetic conversions involved as understood by those of skill in the art. R^{5a} and R^{7a} are understood by those of skill in the art to be synthetic precursors of R⁵ and R⁷, respectively, as further illustrated in the Examples below. The descriptions of the various chemical groups that can be represented by R^{5a} and R^{7a} are generally the same as for R⁵ and R⁷, respectively, as described elsewhere herein.

### Pharmaceutical Compositions

Some embodiments described herein relate to a pharmaceutical composition, that can include an effective amount of one or more compounds described herein (for example, a compound of Formula (I), or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound of Formula (I), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container and labeled for treatment of an indicated condition.

### Uses and Methods of Treatment

Some embodiments described herein relate to a method for treating a cancer or a tumor described herein that can include administering an effective amount of a compound described herein (for example, a compound of Formula (I), or a pharmaceutically acceptable salt thereof) or a pharmaceutical composition that includes a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes a compound described herein (for example, a compound of Formula (I), or a pharmaceutically acceptable salt thereof) in the manufacture of a medicament for treating a cancer or a tumor described herein. Still other embodiments described herein relate to an effective amount of a compound described herein (for example, a compound of Formula (I), or a pharmaceutically acceptable salt thereof) or a pharmaceutical composition that includes a compound described herein (for example, a compound of Formula (I), or a pharmaceutically acceptable salt thereof) for treating a cancer or a tumor described herein.

Some embodiments described herein relate to a method for inhibiting replication of a malignant growth or a tumor described herein that can include contacting the growth or the tumor with an effective amount of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) in the manufacture of a medicament for inhibiting replication of a malignant growth or a tumor described herein. In some embodiments, the use can include contacting the growth or the tumor with the medicament. Still other embodiments described herein relate to an effective amount of a compound described herein (for example, a compound of Formula (I), or a pharmaceutically acceptable salt thereof) for inhibiting replication of a malignant growth or a tumor described herein.

Some embodiments described herein relate to a method for treating a cancer described herein that can include contacting a malignant growth or a tumor described herein with an effective amount of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) in the manufacture of a medicament for treating a cancer described herein. In some embodiments, the use can include contacting the malignant growth or a tumor described herein with the medicament. Still other embodiments described herein relate to an effective amount of a compound described herein (for example, a compound of Formula (I), or a pharmaceutically acceptable salt thereof) for contacting a malignant growth or a tumor described herein, wherein the malignant growth or tumor is due to a cancer described herein.

Examples of suitable malignant growths, cancers and tumors include, but are not limited to: bladder cancers, brain cancers, breast cancers, bone marrow cancers, cervical cancers, colorectal cancers, esophageal cancers, hepatocellular cancers, lymphoblastic leukemias, follicular lymphomas, lymphoid malignancies of T-cell or B-cell origin, melanomas, myelogenous leukemias, Hodgkin's lymphoma, Non-Hodgkin's lymphoma, head and neck cancers (including oral cancers), ovarian cancers, non-small cell lung cancer, chronic lymphocytic leukemias, myelomas (including multiple myelomas), prostate cancer, small cell lung cancer, spleen cancers, polycythemia vera, thyroid cancers, endometrial cancer, stomach cancers, gallbladder cancer, bile duct cancers, testicular cancers, neuroblastomas, osteosarcomas, Ewings's tumor and Wilm's tumor.

As described herein, a malignant growth, cancer or tumor, can become resistant to one or more anti-proliferative agents. In some embodiments, a compound described herein (for example, a compound of Formula (I), or a pharmaceutically acceptable salt thereof) or a pharmaceutical composition that includes a compound described herein (for example, a compound of Formula (I), or a pharmaceutically acceptable salt thereof) can be used to treat and/or ameliorate a malignant growth, cancer or tumor, that has become resistant to one or more anti-proliferative agents (such as one or more Bcl-2 inhibitors). Examples of anti-proliferative agents that a subject may have developed resistance to include, but are not limited to, Bcl-2 inhibitors (such as venetoclax, navitoclax, obatoclax, 555746, APG-1252, APG-2575 and ABT-737). In some embodiments, the malignant growth, cancer or tumor, that has become resistant to one or more anti-proliferative agents can be a malignant growth, cancer or tumor, described herein.

Some embodiments described herein relate to a method for inhibiting the activity of Bcl-2 (such as by, for example, inhibiting the activity of a Bcl-2 protein and/or a Bcl-xL protein) that can include administering an effective amount of a compound described herein (for example, a compound of Formula (I), or a pharmaceutically acceptable salt thereof) or a pharmaceutical composition that includes a compound described herein (for example, a compound of Formula (I), or a pharmaceutically acceptable salt thereof) to a subject and can also include contacting a cell that expresses Bcl-2 with an effective amount of a compound described herein (for example, a compound of Formula (I), or a pharmaceutically acceptable salt thereof) or a pharmaceutical composition that includes a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) in the manufacture of a medicament for inhibiting the activity of Bcl-2 in a subject (such as by, for example, inhibiting the activity of a Bcl-2 protein and/or a Bcl-xL protein) or, in the manufacture of a medicament for inhibiting the activity of Bcl-2 (such as by, for example, inhibiting the activity of a Bcl-2 protein and/or a Bcl-xL protein), wherein the use comprises contacting with a cell that expresses Bcl-2. Still other embodiments described herein relate to an effective amount of a compound described herein (for example, a compound of Formula (I), or a pharmaceutically acceptable salt thereof) for inhibiting the activity of Bcl-2 in a subject (such as by, for example, inhibiting the activity of a Bcl-2 protein and/or a Bcl-xL protein); or for inhibiting the activity of Bcl-2 (such as by, for example, inhibiting the activity of a Bcl-2 protein and/or a Bcl-xL protein) by contacting with a cell that expresses Bcl-2.

In some embodiments, the Bcl protein inhibitor of Formula (I) can be a selective Bcl-2 inhibitor, a selective Bcl-X_{L} inhibitor, a selective Bcl-W inhibitor, a selective Mcl-1 inhibitor or a selective Bcl-2A1 inhibitor. In some embodiments, the Bcl protein inhibitor of Formula (I) can inhibit more than one Bcl protein. In some embodiments, the Bcl protein inhibitor can be an inhibitor of the activity of Bcl-2 and one, two or three of Bcl-X_{L}, Bcl-W, Mcl-1 and Bcl-2A1. In some embodiments, the Bcl protein inhibitor can be an inhibitor of the activity of Bcl-X_{L} and one, two or three of Bcl-W, Mcl-1 and Bcl-2A1. In some embodiments, the Bcl protein inhibitor of Formula (I) can inhibit Bcl-2 and/or Bcl-X_{L}. In some embodiments, the Bcl protein inhibitor of Formula (I) can inhibit both Bcl-2 and Bcl-X_{L}.

Several known Bcl-2 inhibitors can cause one or more undesirable side effects in the subject being treated. Examples of undesirable side effects include, but are not limited to, thrombocytopenia, neutropenia, anemia, diarrhea, nausea and upper respiratory tract infection. In some embodiments, a compound described herein (for example, a compound of Formula (I), or a pharmaceutically acceptable salt thereof) can decrease the number and/or severity of one or more side effects associated with a known Bcl-2 inhibitors. In some embodiments, a compound of Formula (I), or a pharmaceutically acceptable salt thereof, can result in a severity of a side effect (such as one of those described herein) that is 25% less than compared to the severity of the same side effect experienced by a subject receiving a known Bcl-2 inhibitors (such as venetoclax, navitoclax, obatoclax, ABT-737, S55746, AT-101, APG-1252 and APG-2575). In some embodiments, a compound of Formula (I), or a pharmaceutically acceptable salt thereof, results in a number of side effects that is 25% less than compared to the number of side effects experienced by a subject receiving a known Bcl-2 inhibitors (for example, venetoclax, navitoclax, obatoclax, ABT-737, S55746, AT-101, APG-1252 and APG-2575). In some embodiments, a compound of Formula (I), or a pharmaceutically acceptable salt thereof, results in a severity of a side effect (such as one of those described herein) that is less in the range of about 10% to about 30% compared to the severity of the same side effect experienced by a subject receiving a known Bcl-2 inhibitors (for example, venetoclax, navitoclax, obatoclax, ABT-737, S55746, AT-101, APG-1252 and APG-2575). In some embodiments, a compound of Formula (I), or a pharmaceutically acceptable salt thereof, results in a number of side effects that is in the range of about 10% to about 30% less than compared to the number of side effects experienced by a subject receiving a known Bcl-2 inhibitors (for example, venetoclax, navitoclax, obatoclax, ABT-737, S55746, APG-1252 and APG-2575).

The one or more compounds of Formula (I), or a pharmaceutically acceptable salt thereof, that can be used to treat, ameliorate and/or inhibit the replication of a cancer, malignant growth, or tumor wherein inhibiting the activity of Bcl-2 is beneficial is provided in any of the embodiments described above under the heading titled “Compounds.” For example, in various embodiments, the methods and uses described above in the Uses and Methods of Treatment section of this disclosure are carried out in the described manner (generally involving cancer, malignant growth, and/or tumor) using a compound of Formula (I), or a pharmaceutically acceptable salt thereof.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, that elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer), a therapeutically effective amount is that amount that alleviates or eliminates cough, shortness of breath and/or pain. As another example, an effective amount, or a therapeutically effective amount of a Bcl-2 inhibitor is the amount which results in the reduction in Bcl-2 activity and/or an increase in apoptosis. Methods for measuring reductions in Bcl-2 activity are known to those skilled in the art and can be determined by the analysis of Bcl-2 binding and/or degradation, and/or relative levels of cells undergoing apoptosis.

The amount of the compound of Formula (I), or a pharmaceutically acceptable salt thereof, required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of Formula (I), or pharmaceutically acceptable salts thereof, can be determined by comparing their in vitro activity and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Intermediate 1

### 2-(Diethoxymethyl)-5,5-dimethylcyclohexan-1-one

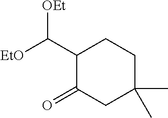 [SMILES: CCOC(OCC)C1CCC(C)(C)CC1=O]

To a solution of triethyl orthoformate (1.32 L, 7.923 mol) in DCM (8.0 L) at −30° C. was added BF₃.OEt₂ (1.244 L, 9.9 mmol) dropwise over 30 min. The reaction mixture was warmed to 0° C. and stirred for 30 min. The reaction mixture was then cooled to −78° C. and 3,3-dimethylcyclohexanone (500 g, 3.96 mol) and N,N-diisopropylethylamine (2.08 L, 11.9 mol) were added dropwise and the reaction was stirred for 2 h at the same temperature. The reaction was then carefully poured into a mixture of sat. aq. NaHCO₃ (25 L) and DCM (10 L). The resulting mixture was stirred for 15 min at rt and the organic layer was separated. The aqueous layer was extracted with DCM (2×10 L) and the combined organic layers were washed with 10% NaCl(aq.) (5 L), dried over Na₂SO₄, filtered and concentrated. The crude product was purified by column chromatography (SiO₂, EtOAc/pet. ether) to afford Intermediate 1 (750 g, 83% yield) as a pale yellow oil. ¹H NMR (400 MHz, CDCl₃) δ 4.83 (d, J=6.0 Hz, 1H), 3.73-3.57 (m, 4H), 2.56-2.53 (m, 1H), 2.20-2.14 (m, 2H), 2.11-2.10 (m, 1H), 1.81 (m, 1H), 1.62-1.56 (m, 2H), 1.21-1.17 (m, 6H), 1.01 (s, 3H), 0.91 (s, 3H).

### Intermediate 2

### 4,4-dimethyl-2-(3-methylbicyclo[1.1.1]pentan-1-yl)cyclohex-1-ene-1-carbaldehyde

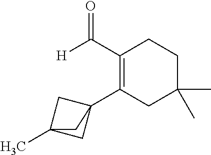

Step 1: A solution of 1-iodo-3-methylbicyclo[1.1.1]pentane (30 g, 144.20 mmol) in THF (225 mL) was cooled to −78° C. and sec-butyllithium (1.4M in cyclohexane, 154.50 mL, 216.30 mmol) was added drop wise over 1 h. The resulting pale yellow suspension was stirred at −78° C. for 10 min and then warmed to 0° C. and stirred for 80 min. The reaction mixture was then cooled to −78° C., and a solution of Intermediate 1 (24.67 g, 108.15 mmol) in THF (75 mL) was added drop wise over 20 min. After 10 min, the reaction was warmed to 0° C. for 1 h. The reaction mixture was then quenched with sat. aq. NH₄C₁ (300 mL) and extracted with Et₂O (2×450 mL). The combined organic layers were dried over Na₂SO₄, filtered and concentrated to afford 2-(diethoxymethyl)-5,5-dimethyl-1-(3-methylbicyclo[1.1.1]pentan-1-yl)cyclohexan-1-ol (Intermediate 2-1) (31 g, crude) as a pale yellow oil. This was used in the next step without further purification.

Step 2: A solution of Intermediate 2-1 (62 g, 199.69 mmol) in 1,4-dioxane (1.24 L), was treated with 2N HCl(aq.) (299.5 mL, 599.2 mmol) at rt and then warmed to 70° C. After 16 h, the reaction was cooled to rt, poured into water (1.24 L) and extracted with Et₂O (2×750 mL). The combined organic layers were dried over Na₂SO₄, filtered and concentrated. The crude product was purified by column chromatography (SiO₂, EtOAc/pet. ether) to provide Intermediate 2 (23 g, 36% yield over 2 steps) as a yellow oil. ¹H NMR (400 MHz, CDCl₃): δ 10.28 (s, 1H), 2.25-2.22 (m, 2H), 1.94 (s, 6H), 1.92 (br s, 2H), 1.35-1.32 (m, 2H), 1.19 (s, 3H), 0.90 (s, 6H).

### Intermediate 3

### 2-(3-(difluoromethyl)bicyclo[1.1.1]pentan-1-yl)-4,4-dimethylcyclohex-1-ene-1-carbaldehyde

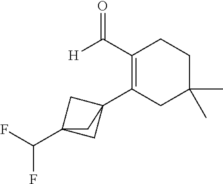 [SMILES: CC1(C)CCC(C=O)=C(C23CC(C(F)F)(C2)C3)C1]

Step 1: Preparation of CF₂HI (based on a procedure from Cao, P. et. al. J. Chem. Soc., Chem. Commun. 1994, 737-738): performed in two parallel batches: A mixture of KI (94 g, 568 mol), MeCN (228 ml) and water (18 mL) was heated to 45° C. and treated with, 2,2-difluoro-2-(fluorosulfonyl)acetic acid (50 g, 284 mmol) in MeCN (50 mL) dropwise over 4 h. The reaction mixture was then cooled to 0° C., and diluted with pentane (150 mL) and water (125 mL). The aqueous layer was washed with pentane (150 mL), and the combined organic layers from both reactions were washed with sat. aq. NaHCO₃ (200 mL), and dried over Na₂SO₄ to obtain 500 mL of difluoromethyl iodide solution. The solution was washed with additional water (2×200 mL) to remove residual acetonitrile, and dried over Na₂SO₄ to obtain difluoroiodomethane (Intermediate 3-1) (0.15 M in pentane, 400 mL, 11% yield). ¹H NMR (400 MHz, CDCl₃) δ 7.67 (t, J=56.0 Hz, 1H).

Step 2: To a stirred solution of [1.1.1]propellane (0.53 M in Et₂O, 52 mL, 27.56 mmol) at −40° C. was added Intermediate 3-1 (0.15 M in pentane, 200 mL, 30 mmol). The reaction mixture was warmed to rt, protected from light, and stirred for 2 days. The reaction was then concentrated at 0-10° C. to obtain 1-(difluoromethyl)-3-iodobicyclo[1.1.1]pentane (Intermediate 3-2) (5 g, 20.5 mmol, 74% yield) as a white solid. ¹H NMR (400 MHz, CDCl₃) δ 5.65 (t, J=56.0 Hz, 1H), 2.40 (s, 6H).

Step 3: A solution of Intermediate 3-2 (30 g, 122.94 mmol) in THF (225 mL) was cooled to −78° C. and sec-butyllithium (1.4M in cyclohexane, 219 mL, 306.7 mmol) was added drop-wise for 1 h. The resulting pale yellow suspension was stirred at −78° C. for 10 min and temperature was raised to 0° C. and stirred for 80 min. The reaction mixture was then cooled to −78° C., and a solution of Intermediate 1 (21 g, 92.20 mmol) in THF (75 mL) was added drop wise to the reaction over 20 min. After 10 min, the reaction was warmed to 0° C. for 1 h. The reaction mixture was quenched with sat. aq. NH₄C₁ (450 mL) and extracted with Et₂O (2×300 mL). The combined organic layers were dried over Na₂SO₄, filtered and concentrated to afford 2-(diethoxymethyl)-1-(3-(difluoromethyl)bicyclo[1.1.1]pentan-1-yl)-5,5-dimethylcyclohexan-1-ol (Intermediate 3-3) (31 g, crude) as pale yellow oil. The crude product was used in the next step without further purification.

Step 4: Intermediate 3 was prepared following the procedure described in Step 2 for Intermediate 2 using Intermediate 3-3 in place of Intermediate 2-1 (38% over 2 steps). ¹H NMR (400 MHz, CDCl₃): δ 10.26 (s, 1H), 5.73 (t, J=56.0 Hz, 1H), 2.29-2.25 (m, 2H), 2.18 (s, 6H), 1.94-1.93 (m, 2H), 1.37 (t, J=6.8 Hz, 2H), 0.91 (s, 6H).

### Intermediate 4

### 2-(3-ethylbicyclo[1.1.1]pentan-1-yl)-4,4-dimethylcyclohex-1-ene-1-carbaldehyde

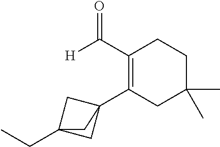 [SMILES: CCC12CC(C3=C(C=O)CCC(C)(C)C3)(C1)C2]

Step 1: To a stirred solution of [1.1.1]propellane (0.19M in Et₂O/pentane), 128.6 mmol) at −78° C. was added EtI (18.7 g, 257.38 mmol). The reaction was warmed to rt and stirred for 3 days in the dark. The reaction was then concentrated at 0° C. to afford 1-ethyl-3-iodobicyclo[1.1.1]pentane (Intermediate 4-1) (21.2 g, 74% yield) as yellow oil. ¹H NMR (400 MHz, CDCl₃) δ 2.17 (s, 6H), 1.52 (q, J=8.0 Hz, 2H), 0.84 (t, J=7.2 Hz, 3H).

Step 2: To a stirred solution of Intermediate 4-1 (10.90 g, 49.1 mmol) in Et₂O (75 mL) at −78° C. was added sec-BuLi (1.4 M in cyclohexane, 50 mL, 70.0 mmol). After 10 min, the reaction was warmed to rt and stirred for 1 h. The reaction mixture was then cooled to −78° C. and treated with a solution of 2-(diethoxymethyl)-5,5-dimethylcyclohexan-1-one (8 g, 35.0 mmol) in Et₂O (25 mL). After 1 h, the reaction was warmed to 0° C. and stirred for 2 h. The reaction was quenched with sat. aq. NH₄C₁ (20 mL) and extracted with EtOAc (3×70 mL). The combined organic layers were then dried over Na₂SO₄, filtered and concentrated to provide 8.5 g of crude 2-(diethoxymethyl)-1-(3-ethylbicyclo[111]pentan-1-yl)-5,5-dimethylcyclohexan-1-ol (Intermediate 4-2). This was used in the next step without further purification.

Step 3: A solution of Intermediate 4-2 (8.5 g, crude) in acetone (80 mL), was treated with 2N HCl(aq.) (20 mL) at rt and then warmed to 75° C. After 24 h, the reaction was concentrated and then diluted with water (50 mL) and extracted with Et₂O (3×250 mL). The combined organic layers were washed with sat. aq. NaHCO₃, dried over Na₂SO₄ and concentrated. The crude product was purified by column chromatography (SiO₂, Et₂O/pet. ether) to provide Intermediate 4 (3.9 g, 48% yield over 2 steps) as a brown oil. ¹H NMR (400 MHz, CDCl₃) δ 10.30 (s, 1H), 2.26-2.22 (m, 2H), 1.93-1.92 (m, 2H), 1.89 (s, 6H), 1.49 (q, J=7.2 Hz, 2H), 1.33 (t, J=6.4 Hz, 2H), 0.89 (s, 6H), 0.87 (t, J=7.6 Hz, 3H).

### Intermediate 5

### 4-(4-((4,4-Dimethyl-2-(3-methylbicyclo[1.1.1]pentan-1-yl)cyclohex-1-en-1-yl)methyl)piperazin-1-yl)benzoic acid

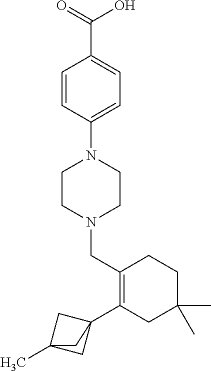

Step 1: To a stirred solution of methyl 4-(piperazin-1-yl)benzoate (1.68 g, 7.6 mmol) and Intermediate 2 (2.0 g, 9.15 mmol) in THF (20 mL) was added Na(OAc)₃BH (4.8 g, 22.8 mmol) at rt. After 16 h, the reaction was put in an ice batch and quenched with sat. aq. NaHCO₃ (25 mL). The reaction mixture was extracted with EtOAc (3×50 mL), dried over Na₂SO₄, filtered, and concentrated. The crude product was purified by column chromatography (SiO₂, EtOAc/pet. ether) to obtain methyl 4-(4-((4,4-dimethyl-2-(3-methylbicyclo[1.1.1]pentan-1-yl)cyclohex-1-en-1-yl)methyl)piperazin-1-yl)benzoate (Intermediate 5-1) as a white solid (1.5 g, 46% yield). LC/MS (ESI) m/z 423.2[M+H]⁺.

Step 2: Step 2: To a stirred solution of Intermediate 5-1 (500 mg, 1.18 mmol) in MeOH:THF:H₂O (1:1:1) (6 mL) was added LiOH.H₂O (148 mg, 3.4 mmol) at rt. The reaction was heated to 30° C. and stirred for 16 h. The volatile solvents were then removed, and the reaction was neutralized with 1N HCl and extracted with 95:5 DCM:MeOH (3×25 mL). The combined organic layers were dried over Na₂SO₄, filtered and concentrated to provide Intermediate 5 (350 mg, 73% yield) as a white solid. ¹H NMR (300 MHz, DMSO-d₆) δ 12.25 (br s, 1H), 7.75 (d, J=9.0 Hz, 2H), 6.95 (d, J=9.0 Hz, 2H), 3.32-3.25 (m, 4H), 3.03 (s, 2H), 2.45-2.35 (m, 4H), 2.06-2.04 (m, 2H), 1.79 (s, 6H), 1.68 (s, 2H), 1.26 (t, J=6.3 Hz, 2H), 1.12 (s, 3H), 0.85 (s, 6H); LC/MS (ESI) m/z 409.5 [M+H]⁺.

### Intermediate 6

### 4-(4-((2-(3-(Difluoromethyl)bicyclo[1.1.1]pentan-1-yl)-4,4-dimethylcyclohex-1-en-1-yl)methyl)piperazin-1-yl)benzoic acid

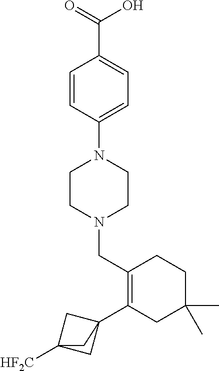

Step 1: Methyl 4-(4-((2-(3-(difluoromethyl)bicyclo[1.1.1]pentan-1-yl)-4,4-dimethylcyclohex-1-en-1-yl)methyl)piperazin-1-yl)benzoate (Intermediate 6-1) was prepared following the procedure described in Step 1 for Intermediate 5 using Intermediate 3 in place of Intermediate 2. LC/MS (ESI) m/z 459.6 [M+H]⁺.

Step 2: Intermediate 6 was prepared following the procedure described in Step 2 for Intermediate 5 using Intermediate 6-1 in place of Intermediate 5-1. LC/MS (ESI) m/z 445.6 [M+H]⁺.

### Intermediate 7

### 4-(4-((2-(3-ethylbicyclo[1.1.1]pentan-1-yl)-4,4-dimethylcyclohex-1-en-1-yl)methyl)piperazin-1-yl)benzoic acid

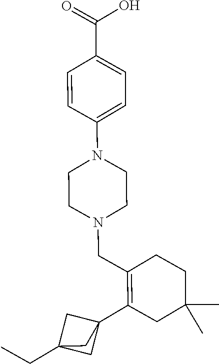 [SMILES: CCC12CC(C3=C(CN4CCN(c5ccc(C(=O)O)cc5)CC4)CCC(C)(C)C3)(C1)C2]

Step 1: Methyl 4-(4-((2-(3-ethylbicyclo[1.1.1]pentan-1-yl)-4,4-dimethylcyclohex-1-en-1-yl)methyl)piperazin-1-yl)benzoate (Intermediate 7-1) was prepared following the procedure described in Step 1 for Intermediate 5 using Intermediate 4 in place of Intermediate 2. LC/MS (ESI) m/z 437.3 [M+H]⁺.

Intermediate 7 was prepared following the procedure described in Step 2 for Intermediate 5 using Intermediate 7-1 in place of Intermediate 5-1. LC/MS (ESI) m/z 423.3 [M+H]⁺.

### Intermediate 8

### (R)-4-(4-(4-hydroxypiperidin-1-yl)-1-(phenylthio)butan-2-ylamino)-3-(trifluoromethylsulfonyl)benzenesulfonamide

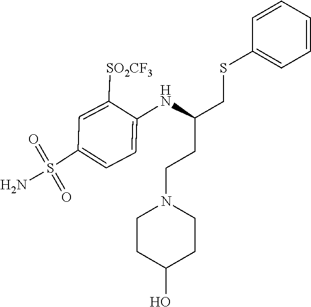

Step 1: To a stirred solution of (R)-3-((((9H-fluoren-9-yl)methoxy)carbonyl)amino)-4-(phenylthio)butanoic acid (6.8 g, 15.7 mmol) in DCM (70 mL) and DMF (10 mL) was added HATU (9.5 g, 25.12 mmol) followed by DIPEA (8.3 mL, 47.1 mmol) at 0° C. After 10 min, 4-hydroxypiperidine (2.4 g, 23.55 mmol) was added and temperature was raised to rt. After 16 h, the reaction was diluted with water and extracted with EtOAc. The combined organic layers were dried over Na₂SO₄, filtered, and concentrated. The crude product was purified by column chromatography (SiO₂ MeOH/DCM) to afford (R)-(9H-fluoren-9-yl)methyl-4-(4-hydroxypiperidin-1-yl)-4-oxo-1-(phenylthio)butan-2-ylcarbamate (Intermediate 8-1) (5.5 g, 68% yield) as a brown oil. LC/MS (ESI) m/z 517.6 [M+H]⁺.

Step 2: To a stirred solution of Intermediate 8-1 (2.75 g, 5.32 mmol) in CH₃CN (20 mL) at rt was added diethylamine (3.3 mL, 31.92 mmol) and stirred at rt. After 16 h, the reaction was concentrated and purified by column chromatography (neutral alumina, MeOH/DCM) to afford (R)-3-amino-1-(4-hydroxypiperidin-1-yl)-4-(phenylthio)butan-1-one (Intermediate 8-2) (900 mg, 57% yield) as a brown liquid. LC/MS (ESI) m/z 295.1 [M+H]⁺.

Step 3: To a stirred solution of Intermediate 8-2 (0.9 g, 3.06 mmol) in anhydrous THF (12 mL) at 0° C. was added BH3 (1 M in THF, 9.18 mL, 9.18 mmol) and the temperature was raised to 45° C. After 16 h, the reaction was cooled to 0° C. and MeOH (30 ml) was added. After 1 hour, the reaction was concentrated and purified by column chromatography (C₁₈, CH₃CN/Water) to afford (R)-1-(3-amino-4-(phenylthio)butyl)piperidin-4-ol (Intermediate 8-3) (305 mg, 36% yield) as an off-white semi solid. LC/MS (ESI) m/z 281.2 [M+H]⁺.

Step 4: To a stirred solution of Intermediate 8-3 (100 mg, 0.357 mmol) in DMF (1 mL) was added 4-fluoro-3-(trifluoromethylsulfonyl)benzenesulfonamide (99 mg, 0.32 mmol) followed by DIPEA (140 mg, 1.07 mmol) and the resulting reaction mixture was stirred at rt. After 16 h, the reaction was concentrated, diluted with water and extracted with 9:1 DCM:MeOH (2×10 mL). The combined organic layers were dried over Na₂SO₄, filtered and concentrated. The crude product was purified by trituration with EtOAc/Et₂O to afford Intermediate 8 (105 mg, 51% yield) as a white solid. LC/MS (ESI) m/z 568.1 [M+H]⁺.

### Intermediate 9

### tert-butyl (R)-4-(4-(phenylthio)-3-((4-sulfamoyl-2-((trifluoromethyl)sulfonyl)phenyl)amino)butyl)piperazine-1-carboxylate

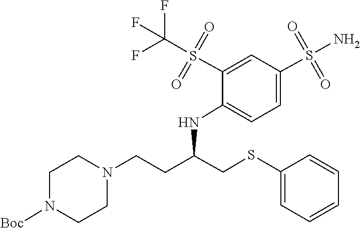 [SMILES: CC(C)(C)OC(=O)N1CCN(CC[C@H](CSc2ccccc2)Nc2ccc(S(N)(=O)=O)cc2S(=O)(=O)C(F)(F)F)CC1]

Step 1: To a stirred solution of (R)-4-(phenylthio)-3-((4-sulfamoyl-2-((trifluoromethyl)sulfonyl)phenyl)amino)butanoic acid (prepared following a procedure described in patent WO2012017251A1) (500 mg, 1.0 mmol), DMAP (122 mg, 1.0 mmol), and EDC.HCl (288 mg, 1.50 mmol) in DCM (10 mL) was added tert-butyl piperazine-1-carboxylate (220 mg, 1.20 mmol) and Et₃N (0.28 mL, 2.00 mmol) at rt. After 15 min, the reaction was heated to 35° C. and stirred for 16 h. The reaction mixture was cooled to rt, diluted with DCM (50 mL) and MeOH (5 mL) and washed with 10% CH₃CO₂H (aq.) (2×15 mL). The organic layer was washed with 5% NaHCO₃ (aq.) (2×10 mL) and 5% NaCl(aq.) (2×10 mL) and concentrated. The crude product was purified by column chromatography (SiO₂, DCM/MeOH) to afford (R)-tert-Butyl 4-(4-(phenylthio)-3-((4-sulfamoyl-2-((trifluoromethyl)sulfonyl)phenyl)-amino)butanoyl)piperazine-1-carboxylate (Intermediate 9-1) (420 mg, 62% yield). LC/MS (ESI) m/z 665.4 [M−H]⁻.

Step 2: To a stirred solution of Intermediate 9-1 (300 mg, 0.45 mmol) in THF (30 mL) was added BH3.THF (1M in THF, 2.25 mL, 2.25 mmol) at 0° C. The resulting reaction mixture was heated to 55° C. for 16 h in a sealed tube. The reaction was then cooled to 0° C., and treated with MeOH (4 mL) and heated to 40° C. After 12 h. the reaction was concentrated and the crude product was purified by column chromatography (C18, DCM/MeOH) to afford Intermediate 9 (150 mg, 51% yield). LC/MS (ESI) m/z 653.2 [M+H]⁺.

### General Procedure A: Acyl Sulfonamide Formation

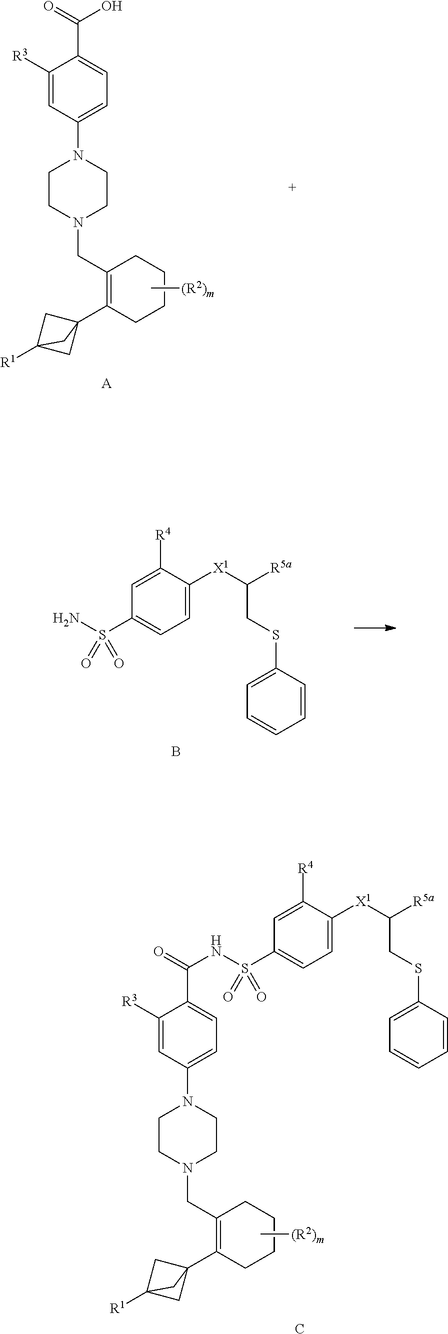

To a solution of corresponding sulfonamide B (1.0 equiv) in DCM (0.01-0.1 M) at rt was added EDC.HCl (1.5-1.75 equiv.) and DMAP (1-2.5 equiv.). In a separate flask, the appropriate acid A (1-1.1 equiv.) was dissolved in DCM (0.02-0.1M) and treated with Et₃N (2-2.5 equiv). (Notes #1 and 2). The acid solution was added to the sulfonamide suspension and either stirred at rt and/or heated to 35° C. Upon completion as determined by LCMS, N,N-dimethylethylenediamine (2-2.5 equiv., Note #3) was added to the reaction mixture and the reaction was stirred for 90 min. The reaction mixture was then washed with 10% aq. AcOH (Note #4), 5% NaHCO₃ (aq.) and then with 5% NaCl (aq.). The organic layer was dried, filtered and concentrated. The crude product C was either purified by 1) column chromatography (SiO₂), 2) HPLC (10 mM NH₄CO₃H(aq): CH₃CN or MeOH), or 3) trituration with an organic solvent.

Note #1: In some instances, DCM was not added.

Note #2: In some instances, Et₃N was added to the flask containing sulfonamide B.

Note #3: In some instances, N,N-dimethylethylenediamine was not added during the workup.

Note #4: In some instances, the organic layer was diluted with DCM and MeOH to solubilize the crude product.

### Intermediate 10

### (R)-4-(4-((4,4-dimethyl-2-(3-methylbicyclo[1.1.1]pentan-1-yl)cyclohex-1-en-1-yl)methyl)piperazin-1-yl)-N-((4-((4-(4-hydroxypiperidin-1-yl)-1-(phenylthio)butan-2-yl)amino)-3-((trifluoromethyl) sulfonyl)phenyl) sulfonyl)benzamide

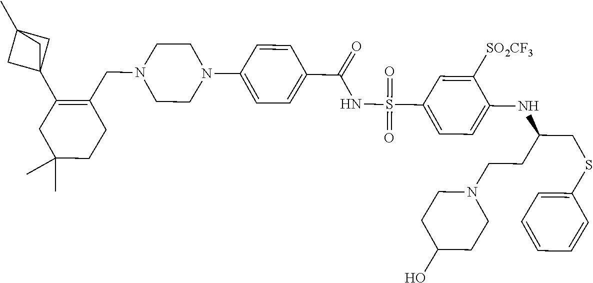

Representative example of General Procedure A: To a stirred solution of Intermediate 8 (138.9 mg, 0.24 mmol), DMAP (29.9 mg, 0.245 mmol), EDC.HCl (70.3 mg, 0.37 mmol) in DCM (5 mL), was added a mixture of Intermediate 5 (100 mg, 0.24 mmol) and Et₃N (68 μL, 0.49 mmol) at rt. The resulting reaction mixture was stirred at rt and then heated to 35° C. and stirred for 16 h. The reaction mixture was cooled to rt, diluted with DCM (48.5 mL) and MeOH (2.5 mL), washed with 10% AcOH(aq.) (2×100 mL), 5% NaHCO₃ (aq.) (2×10 mL), and 5% NaCl(aq.) (2×10 mL). The organic layer was dried over Na₂SO₄, filtered and concentrated. The crude product was purified by HPLC (30:70 to 0:100 10 mM NH₄CO₃H(aq.)/CH₃CN) to afford Intermediate 10 (55 mg, 23% yield) as an off-white solid. LC/MS (ESI) m/z 958.2 [M+H]⁺.

### Intermediate 11

### (R)-4-(4-((2-(3-(Difluoromethyl)bicyclo[1.1.1]pentan-1-yl)-4,4-dimethylcyclohex-1-en-1-yl)methyl)piperazin-1-yl)-N-((4-((4-(4-hydroxypiperidin-1-yl)-1-(phenylthio)butan-2-yl)amino)-3-((trifluoromethyl) sulfonyl)phenyl) sulfonyl)benzamide

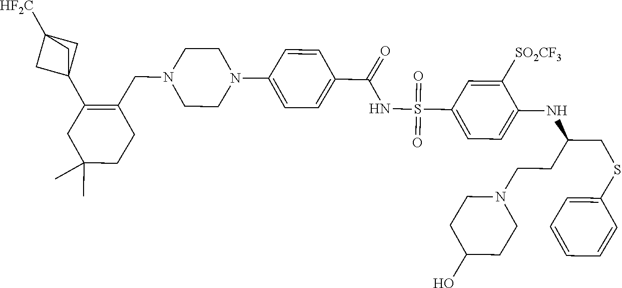

Intermediate 11 was prepared following General Procedure A using Intermediate 6 and Intermediate 8. LC/MS (ESI) m/z 994.6 M+Hr.

### Intermediate 12

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide

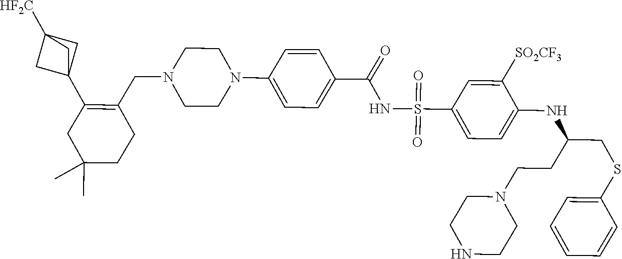

Step 1: (R)-tert-Butyl 4-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoro-methyl)sulfonyl)phenyl)amino)-4-(phenylthio)butyl)piperazine-1-carboxylate (Intermediate 12-1) was prepared following General Procedure A using Intermediate 6 and Intermediate 9. LC/MS (ESI) m/z 1079.3 [M+H]⁺

Step 2: To a stirred solution of Intermediate 12-1 (350 mg, 0.32 mmol) in Et₂O (5 mL) at 0° C., was added HCl (2M in Et₂O, 2.0 mL). The reaction was warmed to rt and stirred for 16 h. The reaction was concentrated, diluted with ice cold water, treated with sat. aq. NaHCO₃ (10 mL) and extracted with 10% MeOH in DCM (3×30 mL). The combined organic layers were dried over anhydrous Na₂SO₄, filtered and concentrated. The crude product was purified by HPLC (30:70 to 1:99 10 mM NH₄CO₃H(aq.)/CH₃CN) to provide Intermediate 12 (14 mg, 4% yield) as an off-white solid. ¹H NMR (400 MHz, DMSO-d₆) δ 8.32 (br s, 2H), 8.02 (s, 1H), 7.91 (d, J=8.8 Hz, 1H), 7.68 (d, J=8.8 Hz, 2H), 7.34-7.23 (m, 4H), 7.19-7.15 (m, 1H), 6.83-6.75 (m, 3H), 6.66 (d, J=8.8 Hz, 1H), 5.97 (t, J=56.8 Hz, 1H), 3.97 (br s, 1H), 3.26-3.23 (m, 2H), 3.15-3.10 (m, 4H), 3.02-2.90 (m, 6H), 2.52-2.50 (m, 2H), 2.40-2.23 (m, 8H), 2.10-1.83 (m, 9H), 1.67 (s, 3H), 1.23 (t, J=6.4 Hz, 2H), 0.82 (s, 6H); LC/MS (ESI) m/z 979.4 [M+H]⁺.

### Intermediate 13

### (R)-4-(4-((4,4-dimethyl-2-(3-methylbicyclo[1.1.1]pentan-1-yl)cyclohex-1-en-1-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide

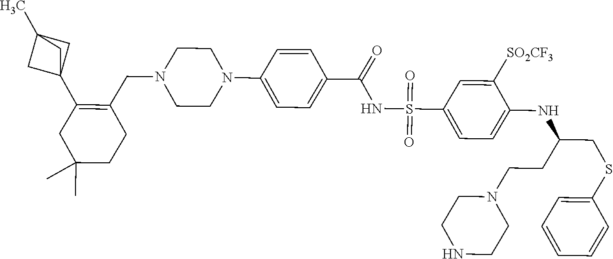

Step 1: tert-butyl (R)-4-(3-((4-(N-(4-(4-((4,4-dimethyl-2-(3-methylbicyclo[1.1.1]pentan-1-yl)cyclohex-1-en-1-yl)methyl)piperazin-1-yl)benzoyl)sulfamoyl)-2-((trifluoromethyl)sulfonyl)phenyl)amino)-4-(phenylthio)butyl)piperazine-1-carboxylate (Intermediate 13-1) was prepared following General Procedure A using Intermediate 5 and Intermediate 9. LC/MS (ESI) m/z 1043.6 [M+H]⁺.

Step 2: To a stirred solution of Intermediate 13-1 (800 mg, 0.767 mmol) in Et₂O (8 mL) was added 2M HCl in Et₂O (8 mL) at 0° C. and the reaction was warmed to rt. After 16 h, the reaction mixture was concentrated and then dissolved in 10% MeOH in DCM (50 mL). The organic layer was washed with sat. aq. NaHCO₃ (2×20 mL), brine (2×20 mL), dried over Na₂SO₄, filtered, and concentrated to afford Intermediate 13 (550 mg, 76% yield) as an off-white solid. ¹H NMR (400 MHz, CDCl₃) δ 8.05 (d, J=2.0 Hz, 1H), 7.94 (dd, J=9.2, 7.2 Hz, 1H), 7.72 (d, J=8.8 Hz, 2H), 7.37-7.35 (m, 2H), 7.31 (t, J=5.6 Hz, 2H), 7.22-7.20 (m, 1H), 6.85-6.79 (m, 3H), 6.69 (d, J=9.2 Hz, 1H), 4.00-3.99 (m, 1H), 3.31-3.23 (m, 4H), 3.15 (s, 4H), 3.01-2.97 (m, 6H), 2.49-2.33 (m, 9H), 2.03-1.99 (m, 3H), 1.79-1.67 (m, 9H), 1.26-1.23 (m, 3H), 1.11 (s, 3H), 0.84 (s, 6H); LC/MS (ESI) m/z 943.5 [M+H]⁺.

### Intermediate 14

### (R)-4-(4-((2-(3-ethylbicyclo[1.1.1]pentan-1-yl)-4,4-dimethylcyclohex-1-en-1-yl)methyl)piperazin-1-yl)-N-((4-((4-(4-hydroxypiperidin-1-yl)-1-(phenylthio)butan-2-yl)amino)-3-((trifluoromethyl)sulfonyl)phenyl)sulfonyl)benzamide

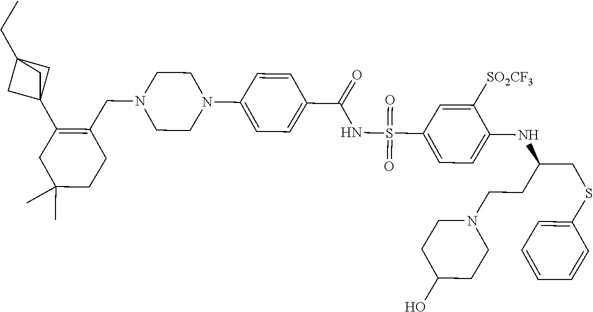

Intermediate 14 is prepared following General Procedure A using Intermediate 7 and Intermediate 8.

### Intermediate 15

### (R)-4-(4-((2-(3-ethylbicyclo[1.1.1]pentan-1-yl)-4,4-dimethylcyclohex-1-en-1-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide

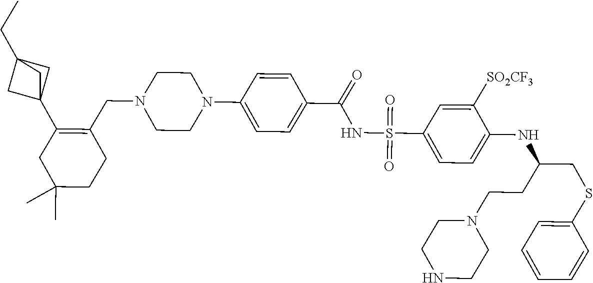

Step 1: tert-butyl (R)-4-(3-((4-(N-(4-(4-((2-(3-ethylbicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperazine-1-carboxylate (Intermediate 15-1) is prepared following General Procedure A using Intermediate 7 and Intermediate 9.

Step 2: Intermediate 15 is prepared following the procedure described in Step 2 for Intermediate 12 using Intermediate 15-1 in place of Intermediate 12-1.

### Intermediate 16

### 2-(2,6-dioxopiperidin-3-yl)-4-((2-(2-(prop-2-yn-1-yloxy)ethoxy)ethyl)amino)isoindoline-1,3-dione

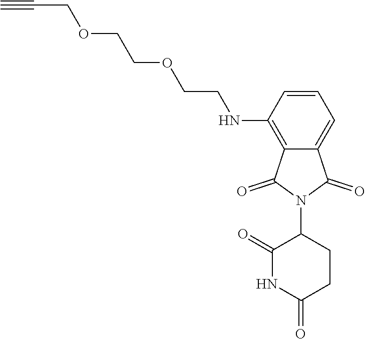 [SMILES: C#CCOCCOCCNc1cccc2c1C(=O)N(C1CCC(=O)NC1=O)C2=O]

To a solution of 2-[2-(2-propyn-1-yloxy)ethoxy]ethanamine (116.6 mg, 0.81 mmol) in 1-methyl-2-pyrrolidinone (3 mL) was added DIPEA (210.6 mg, 1.63 mmol) and 2-(2,6-dioxopiperidin-3-yl)-4-fluoroisoindoline-1,3-dione (150 mg, 0.54 mmol) at rt. The reaction mixture was then heated to 80° C. After 12 h, the reaction mixture was cooled to rt, and water (20 mL) was added to the reaction mixture. The reaction was extracted with EtOAc and the combined organic layers were dried over Na₂SO₄, filtered and concentrated. The crude product was purified by column chromatography (SiO₂) to provide Intermediate 16 (0.12 g, 55% yield). ¹H NMR (400 MHz, CDCl₃) δ 7.93 (s, 1H), 7.55-7.47 (m, 1H), 7.12 (d, J=7.0 Hz, 1H), 6.94 (d, J=8.5 Hz, 1H), 6.49 (s, 1H), 4.96-4.89 (m, 1H), 4.92 (dd, J=12.1, 5.4 Hz, 1H), 4.22 (d, J=2.3 Hz, 2H), 3.75-3.70 (m, 6H), 3.50 (q, J=5.4 Hz, 2H), 2.88-2.76 (m, 3H), 2.44 (t, J=2.4 Hz, 1H), 2.18-2.10 (m, 1H).

### Intermediate 17

### 4-((2-(2-(2-aminoethoxy)ethoxy)ethyl)amino)-2-(2,6-dioxopiperidin-3-yl)isoindoline-1,3-dione

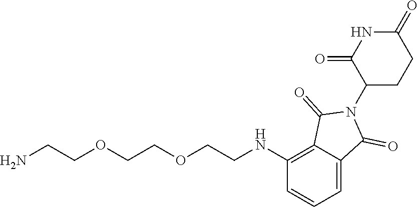 [SMILES: NCCOCCOCCNc1cccc2c1C(=O)N(C1CCC(=O)NC1=O)C2=O]

Step 1: To a solution of 2-(2,6-dioxopiperidin-3-yl)-4-fluoroisoindoline-1,3-dione (2.50, 9.05 mmol), and tert-butyl (2-(2-(2-aminoethoxy)ethoxy)ethyl)carbamate (2.69, 10.9 mmol) in DMSO (25 mL) at rt was added DIPEA (3.23 mL, 18.1 mmol) and the reaction mixture was heated to 90° C. After 16 h, the reaction was cooled to rt and water was added (25 mL). The reaction mixture was extracted with 10% MeOH in DCM (3×75 mL) and the combined organic layers were washed with brine (2×25 mL), dried over Na₂SO₄, filtered and concentrated to provide tert-butyl (2-(2-(2-((2-(2,6-dioxopiperidin-3-yl)-1,3-dioxoisoindolin-4-yl)amino)ethoxy)ethoxy)ethyl)carbamate (Intermediate 17-1) (950 mg, 20% yield) as a yellow solid. LC/MS (ESI) m/z 505.3 [M+H]⁺.

Step 2: A solution of Intermediate 17-1 (500 mg, 1.00 mmol) in DCM (5 mL) was treated with TFA (5 eq.) at 0° C. and then warmed to rt. After 2 h, the reaction mixture was concentrated and then triturated with 20% Et₂O in n-pentane to afford the TFA salt of Intermediate 18 (350 mg, 70% yield) as a colorless oil. LC/MS (ESI) m/z 405.5 [M+H]⁺.

### Intermediate 18

### 2-(2-((2-(2,6-dioxopiperidin-3-yl)-1,3-dioxoisoindolin-4-yl)amino)ethoxy)ethyl methanesulfonate

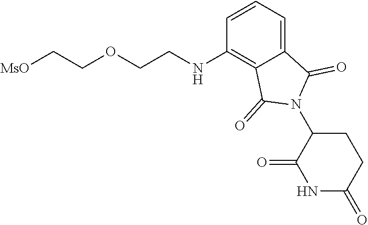 [SMILES: CS(=O)(=O)OCCOCCNc1cccc2c1C(=O)N(C1CCC(=O)NC1=O)C2=O]

Step 1: To a stirred solution of 2-(2,6-dioxopiperidin-3-yl)-4-fluoroisoindoline-1,3-dione (1.0 g, 3.62 mmol) in DMSO (5 mL) was added 2-(2-aminoethoxy)ethan-1-ol (0.571 mg, 5.43 mmol) followed by DIPEA (1.29 mL, 7.29 mmol) at rt. The reaction was heated to 90° C. and stirred for 16 h. The reaction mixture was cooled to rt and purified by column chromatography (SiO₂, MeOH/DCM) to afford 2-(2,6-Dioxopiperidin-3-yl)-4-((2-(2-hydroxyethoxy)ethyl)amino)isoindoline-1,3-dione (Intermediate 18-1) as a yellow solid (530 mg, 40% yield). LC/MS (ESI) m/z 362.3 [M+H]⁺

Step 2: To a stirred solution of Intermediate 18-1 (160 mg, 0.44 mmol) in DCM (4 mL) was added triethylamine (0.43 mL, 3.09 mmol) and MsCl (0.05 mL, 0.75 mmol) at 0° C. and the reaction was warmed to rt. After 3 h, the reaction mixture was diluted with ice cold water, and extracted with DCM (2×20 mL). The combined organic layers were washed with sat. aq. NaHCO₃ (2×5 mL), brine (10 mL), dried over Na₂SO₄ and concentrated to afford Intermediate 18 (190 mg, 97% crude) as a yellow oil. The crude product was used without further purification. ¹H NMR (400 MHz, CDCl₃) δ 8.00 (s, 1H), 7.53-7.49 (m, 1H), 7.12 (d, J=6.8 Hz, 1H), 6.93 (d, J=8.4 Hz, 1H), 6.48 (t, J=5.2 Hz, 1H), 4.93-4.89 (m, 1H), 4.39-4.36 (m, 2H), 3.79-3.72 (m, 4H), 3.51-3.47 (m, 2H), 3.04 (s, 3H), 2.76-2.72 (m, 2H), 2.17-2.12 (m, 2H); LC/MS (ESI) m/z 440.3 [M+H]⁺.

### Intermediate 19

### 2-(2,6-dioxopiperidin-3-yl)-4-((2-(2-(2-iodoethoxy)ethoxy)ethyl)amino)isoindoline-1,3-dione

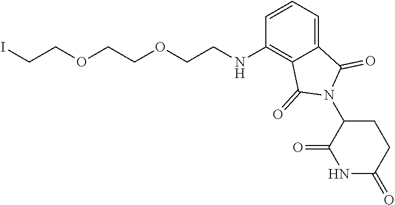 [SMILES: O=C1CCC(N2C(=O)c3cccc(NCCOCCOCCI)c3C2=O)C(=O)N1]

Step 1: 2-(2,6-dioxopiperidin-3-yl)-4-((2-(2-(2-hydroxyethoxy)ethoxy)ethyl)amino)isoindoline-1,3-dione (Intermediate 19-1) was prepared following the procedure described in Step 1 for Intermediate 18 using 2-(2-(2-aminoethoxy)ethoxy)ethanol in place of 2-(2-aminoethoxy)ethan-1-ol. LC/MS (ESI) m/z 406.2 [M+H]⁺.

Step 2: 2-(2-(2-((2-(2,6-dioxopiperidin-3-yl)-1,3-dioxoisoindolin-4-yl)amino)ethoxy)ethoxy)ethyl methanesulfonate (Intermediate 19-2) was prepared following the procedure described in Step 2 for Intermediate 18 using Intermediate 19-1 in place of Intermediate 18-1. LC/MS (ESI) m/z 484.5 [M+H]⁺.

Step 3: A stirred solution of Intermediate 19-2 (350 mg, 0.72 mmol) in CH₃CN (3 mL) was treated with NaI (130 mg, 0.86 mmol) at rt and then heated to 90° C. for 16 h. The reaction mixture was cooled to rt and filtered through Celite. The Celite was washed EtOAc (3×25 mL) and the combined organic layers were concentrated. The crude product was purified by column chromatography (SiO₂, EtOAc/pet. ether) to afford Intermediate 19 (230 mg, 60% yield over two steps) as a yellow solid. LC/MS (ESI) m/z 516.1 [M+H]⁺.

### Intermediate 20

### 2-(2,6-dioxopiperidin-3-yl)-4-((2-(2-(2-(2-iodoethoxy)ethoxy)ethoxy)ethyl)amino)isoindoline-1,3-dione

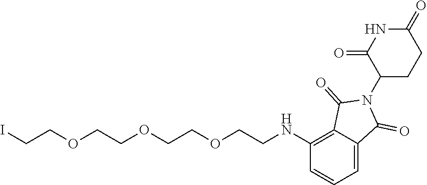 [SMILES: O=C1CCC(N2C(=O)c3cccc(NCCOCCOCCOCCI)c3C2=O)C(=O)N1]

Step 1: 2-(2,6-dioxopiperidin-3-yl)-4-((2-(2-(2-(2-hydroxyethoxy)ethoxy)ethoxy)ethyl)amino)isoindoline-1,3-dione (Intermediate 20-1) was prepared following the procedure described in Step 1 for Intermediate 18 using 2-(2-(2-(2-aminoethoxy)ethoxy)ethoxy)ethan-1-ol in place of 2-(2-aminoethoxy)ethan-1-ol. LC/MS (ESI) m/z 450.1 [M+H]⁺.

Step 2: 2-(2-(2-(2-((2-(2,6-dioxopiperidin-3-yl)-1,3-dioxoisoindolin-4-yl)amino)ethoxy)ethoxy)ethoxy)ethyl methanesulfonate (Intermediate 20-2) was prepared following the procedure described in Step 2 for Intermediate 18 using Intermediate 20-1 in place of Intermediate 18-1. LC/MS (ESI) m/z 528.3 [M+H]⁺.

Step 3: Intermediate 20 was prepared following the procedure described in Step 3 for Intermediate 19 using Intermediate 20-2 in place of Intermediate 19-2. LC/MS (ESI) m/z 560.2 [M+H]⁺.

### Intermediate 21

### tert-butyl (R)-1-(4-(phenylthio)-3-((4-sulfamoyl-2-(trifluoromethyl)sulfonyl)phenyl)amino)butyl)piperidine-4-carboxylate

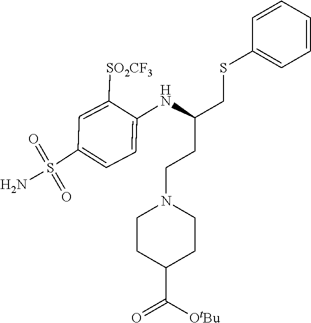

To a stirred solution of tert-butyl piperidine-4-carboxylate (365.2 mg, 1.97 mmol) in THF (15 mL) was added a solution of (R)-4-((4-oxo-1-(phenylthio)butan-2-yl)amino)-3-((trifluoromethyl)sulfonyl)benzenesulfonamide (prepared following a procedure described in WO2012017251A1) (950 mg, 1.97 mmol) in THF (10 mL) at rt. After 1 h, the reaction was cooled to 0° C., and Na(OAc)₃BH (1.25 g, 5.91 mmol) was added, and the reaction was warmed to rt and stirred for 16 h. The reaction mixture was quenched with sat. aq. NaHCO₃ (15 mL) and extracted with EtOAc (2×20 mL). The combined organic layers were washed with brine (15 mL), dried over Na₂SO₄, and concentrated. The crude product was purified by column chromatography (SiO₂, MeOH/DCM) to afford Intermediate 21 (500 mg, 49% yield) as an off-white solid. ¹H NMR (400 MHz, DMSO-d₆) δ 7.98 (d, J=2.4 Hz, 1H), 7.83 (dd, J=9.2, 2.0 Hz, 1H), 7.39-7.29 (m, 6H), 7.22-7.18 (m, 1H), 7.03 (d, J=9.6 Hz, 1H), 6.93 (d, J=8.8 Hz, 1H), 4.09-4.08 (m, 1H), 3.40-3.22 (m, 2H), 2.78-2.75 (m, 1H), 2.55-2.51 (m, 1H), 2.31-2.08 (m, 3H), 1.97-1.89 (m, 2H), 1.92-1.46 (m, 4H), 1.50-1.44 (m, 2H), 1.46 (s, 9H); LC/MS (ESI) m/z 652.1 [M+H]⁺.

### Intermediate 22

### (R)-1-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperidine-4-carboxylic acid

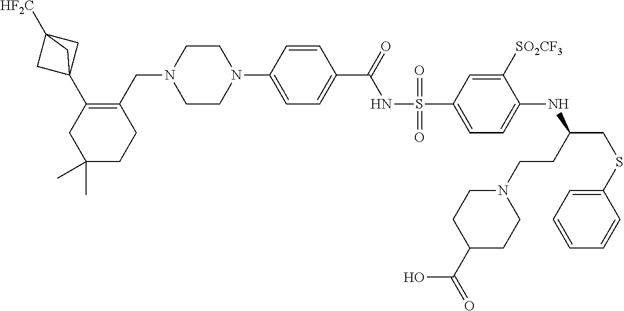

Step 1: tert-butyl (R)-1-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperidine-4-carboxylate (Intermediate 22-1) was prepared following General Procedure A using Intermediate 6 and Intermediate 21. ¹H NMR (400 MHz, CDCl3) δ 8.35 (s, 1H), 8.06 (dd, J=8.8, 1.6 Hz, 1H), 7.72 (d, J=8.4 Hz, 2H), 7.35-7.28 (m, 2H), 7.24-7.22 (m, 3H), 7.01 (d, J=5.6 Hz, 1H), 6.82 (d, J=8.4 Hz, 2H), 6.61 (d, J=9.6 Hz, 1H), 5.81 (t, J=56.8 Hz, 1H), 3.93-3.89 (m, 1H), 3.30 (br s, 4H), 3.06 (br s, 4H), 2.90-2.87 (m, 1H), 2.77-2.74 (m, 1H), 2.50-2.45 (m, 7H), 2.23-2.10 (m, 7H), 2.05 (s, 6H), 1.98-1.89 (m, 3H), 1.78-1.73 (m, 3H), 1.44 (s, 9H), 1.32-1.29 (m, 2H), 0.9 (s, 6H); LC/MS (ESI) m/z 1078.5 [M+H]⁺.

Step 2: To a stirred solution of Intermediate 22-1 (900 mg, 0.834 mmol) in DCM (10 mL) was added TFA (10 mL) at 0° C. The reaction was warmed to rt and stirred for 5 h. The reaction mixture was concentrated and triturated with Et₂₀ and pentane to afford the TFA salt of Intermediate 22 (900 mg) as an off-white solid. LC/MS (ESI) m/z 1022.5 [M+H]⁺.

### Intermediate 23

### 2-(2-((2-(2,6-Dioxopiperidin-3-yl)-1,3-dioxoisoindolin-5-yl)amino)ethoxy)ethyl methanesulfonate

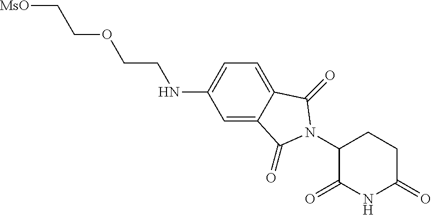 [SMILES: CS(=O)(=O)OCCOCCNc1ccc2c(c1)C(=O)N(C1CCC(=O)NC1=O)C2=O]

Step 1: To a stirred solution of 2-(2,6-dioxopiperidin-3-yl)-5-fluoroisoindoline-1,3-dione(700 mg, 2.53 mmol) in NMP (9 mL) and DMSO (1 mL) at rt was added 2-(2-aminoethoxy)ethan-1-ol (266 mg, 2.53 mmol) followed by DIPEA (652 mg, 5.06 mmol). The reaction was heated to 90° C. and stirred for 12 h. The reaction mixture was cooled to rt, diluted with ice-cold water and extracted with EtOAc (3×50 mL). The combined organic layers were washed with water (2×50 mL), brine (2×10 mL), dried over Na₂SO₄, filtered and concentrated. The crude product was purified by column chromatography (SiO₂, MeOH/DCM) to afford 2-(2,6-dioxopiperidin-3-yl)-5-((2-(2-hydroxyethoxy)ethyl)amino)isoindoline-1,3-dione (Intermediate 23-1) (200 mg, 22% yield) as a off-white solid. ¹H NMR (400 MHz, CDCl₃) δ 7.94 (s, 1H), 7.62 (d, J=8.4 Hz, 1H), 7.01-6.99 (m, 1H), 6.78 (d, J=8.4, 2.0 Hz, 1H), 4.95-4.87 (m, 2H), 3.81-3.74 (m, 4H), 3.64-3.62 (m, 2H), 3.44 (q, J=5.2 Hz, 2H), 2.84-2.73 (m, 3H). 2.17-2.11 (m, 1H), 1.83 (t, J=6.0 Hz, 1H); LC/MS (ESI) m/z 362.5 [M+H]⁺.

Step 2: To a stirred solution of Intermediate 23-1 (160 mg, 0.44 mmol) in DCM (10 mL) was added methanesulfonyl chloride (0.04 mL, 0.531 mmol) and TEA (0.25 mL, 1.77 mmol) at 0° C. The reaction was then warmed to rt and stirred for 2 h. The reaction was quenched with ice-cold water and extracted with DCM (2×30 mL). The combined organic layers were washed with brine (2×10 mL), dried over Na₂SO₄, filtered and concentrated to afford Intermediate 23 (150 mg, 77% crude yield) as a yellow oil. The crude product was used without further purification. LC/MS (ESI) m/z 440.1 [M+H]⁺.

### Intermediate 24

### 2-(2,6-dioxopiperidin-3-yl)-4-(2-(2-iodoethoxy)ethoxy)isoindoline-1,3-dione

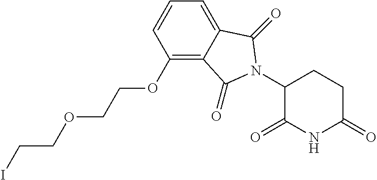 [SMILES: O=C1CCC(N2C(=O)c3cccc(OCCOCCI)c3C2=O)C(=O)N1]

Step 1: To a stirred solution of 2-(2,6-dioxopiperidin-3-yl)-4-hydroxyisoindoline-1,3-dione (0.5 g, 1.82 mmol) in DMF (5 mL) was added NaHCO₃ (0.3 g, 3.64 mmol) followed by KI (0.06 g, 0.364 mmol). After 10 min, 2-(2-chloroethoxy)ethan-1-ol (0.35 g, 2.73 mmol) was added and the resulting reaction mixture was heated to 70° C. and stirred for 12 h. The reaction mixture was then cooled to rt, diluted with ice-cold water, and extracted with 10% MeOH in DCM (3×100 mL). The combined organic layers were washed with water (2×30 mL), brine (2×25 mL), dried over Na₂SO₄, filtered and concentrated. The crude product was purified by purified by column chromatography (SiO₂, EtOAc/pet. ether) to afford 2-(2,6-dioxopiperidin-3-yl)-4-(2-(2-hydroxyethoxy)ethoxy)isoindoline-1,3-dione (Intermediate 24-1) (230 mg, 034% yield) as an off-white oil. LC/MS (ESI) m/z 363.3 [M+H]⁺.

Step 2: 2-(2-((2-(2,6-Dioxopiperidin-3-yl)-1,3-dioxoisoindolin-5-yl)oxy)ethoxy)ethyl methanesulfonate (Intermediate 24-2) was prepared following the procedure described in Step 2 for Intermediate 23 using Intermediate 24-1 in place of Intermediate 23-1. LC/MS (ESI) m/z 441.2 [M+H]⁺.

Step 3: To a stirred solution of Intermediate 24-2 (250 mg, 0.52 mmol) in CH₃CN (5 mL) was added NaI (0.154 mg, 1.04 mmol) and the resulting reaction mixture was heated to 90° C. and stirred for 2 h. The reaction was then cooled to rt, quenched with ice-cold water (50 mL) and extracted with EtOAc (2×100 mL). The combined organic layers were washed with brine (2×25 mL), dried over Na₂SO₄, filtered and concentrated to afford Intermediate 24 (200 mg, 74% crude yield) as a yellow oil. The crude product was used without further purification. LC/MS (ESI) m/z 473.2 [M+H]⁺.

### Intermediate 25

### 2-(2-((2-(2,6-Dioxopiperidin-3-yl)-1,3-dioxoisoindolin-5-yl)oxy)ethoxy)ethyl methanesulfonate

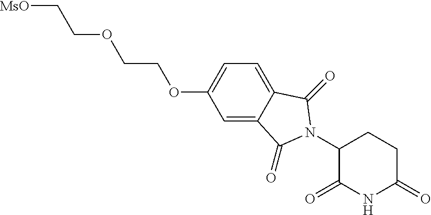 [SMILES: CS(=O)(=O)OCCOCCOc1ccc2c(c1)C(=O)N(C1CCC(=O)NC1=O)C2=O]

Step 1: 2-(2,6-dioxopiperidin-3-yl)-5-(2-(2-hydroxyethoxy)ethoxy)isoindoline-1,3-dione (Intermediate 25-1) was prepared following the procedure described in Step 1 for Intermediate 24 using 2-(2,6-dioxopiperidin-3-yl)-5-hydroxyisoindoline-1,3-dione in place of 2-(2,6-dioxopiperidin-3-yl)-4-hydroxyisoindoline-1,3-dione. LC/MS (ESI) m/z 363.3 [M+H]⁺.

Step 2: Intermediate 25 was prepared following the procedure described in Step 2 for Intermediate 23 using Intermediate 25-1 in place of Intermediate 23-1. LC/MS (ESI) m/z 441.2 [M+H]⁺.

### Intermediate 26

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-(methylamino)-1-(phenylthio)butan-2-yl)amino)-3-((trifluoromethyl)sulfonyl)phenyl)sulfonyl)benzamide

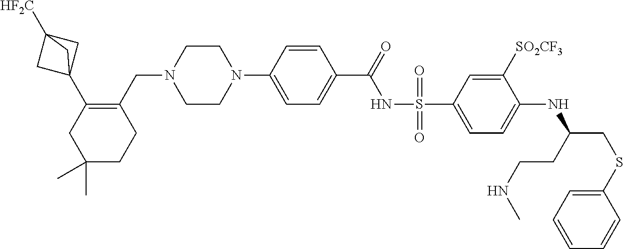

Step 1: Methyl (R)-3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl) sulfonyl)phenyl)amino)-4-(phenylthio)butanoate (Intermediate 26-1) was prepared following General Procedure A using Intermediate 6 and methyl (R)-4-(phenylthio)-3-((4-sulfamoyl-2-((trifluoromethyl)sulfonyl)phenyl)amino)butanoate (prepared following a procedure described in patent WO2012017251A1). ¹H NMR (400 MHz, CDCl₃) δ 8.39 (d, J=2.4 Hz, 1H), 8.15 (dd, J=9.2, 2.0 Hz, 1H), 7.64 (d, J=8.8 Hz, 2H), 7.42 (m, 3H), 7.39-7.28 (m, 3H), 6.81 (d, J=8.8 Hz, 2H), 6.52 (d, J=9.2 Hz, 1H), 5.67 (t, J=56.4 Hz, 1H), 4.10-4.03 (m, 1H), 3.68 (s, 3H), 3.33-3.31 (m, 4H), 3.14-3.08 (m, 4H), 2.80-2.78 (m, 2H), 2.51 (t, J=4.8 Hz, 4H), 2.10-2.04 (m, 2H), 2.01 (s, 6H), 1.70 (s, 2H), 1.32-1.24 (m, 2H), 0.88 (s, 6H), NH proton was not observed; LC/MS (ESI) m/z 939.5 [M+H]⁺.

Step 2: To a stirred solution of Intermediate 26-1 (480 mg, 0.51 mmol) in DCM (40 mL) at −78° C. was added DIBAL-H (1.0 M in toluene, 1.53 mL, 1.53 mmol) drop-wise. After 3 h, the reaction mixture was quenched with MeOH (3 mL) at −78° C., warmed to 0° C., and treated with sat. aq. potassium sodium tartrate (10 mL) and DCM (20 mL). After 1 hr, the organic layer was separated, dried over Na₂SO₄ and concentrated to afford (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-oxo-1-(phenylthio)butan-2-yl)amino)-3-((trifluoromethyl)sulfonyl)phenyl)sulfonyl)benzamide (Intermediate 26-2) (420 mg, 90% crude yield) as an off-white solid. The crude product was used without further purification LC/MS (ESI) m/z 909.52 [M+H]⁺.

Step 3: To a stirred solution of methanamine hydrochloride (62 mg, 0.92 mmol) in THF (10 mL) was added Intermediate 26-2 (420 mg, 0.46 mmol) at rt. After 2 h, the reaction was cooled to 0° C., and Na(OAc)₃BH (293 mg, 1.38 mmol) was added. The reaction mixture was warmed to rt and stirred for 16 h. The reaction mixture was quenched with sat. aq. NaHCO₃ (10 mL), and extracted with EtOAc (3×25 mL). The combined organic layers were dried over Na₂SO₄ and concentrated. The crude product was purified by column chromatography (SiO₂, MeOH/DCM) to afford Intermediate 26 (140 mg, 33% yield) as an off-white solid. LC/MS (ESI) m/z 924.6[M+H]⁺.

### Intermediate 27

### 4-(4-((2-(2-((2-(2,6-dioxopiperidin-3-yl)-1,3-dioxoisoindolin-4-yl)amino)ethoxy)ethoxy)methyl)-1H-1,2,3-triazol-1-yl)butanoic acid

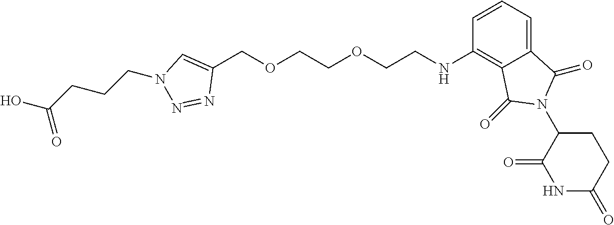 [SMILES: O=C(O)CCCn1cc(COCCOCCNc2cccc3c2C(=O)N(C2CCC(=O)NC2=O)C3=O)nn1]

Step 1: To a solution of Intermediate 16 (220 mg, 0.55 mmol) in MeOH (3 mL) was added CuI (104.9 mg, 0.55 mmol) and tert-butyl 4-azidobutanoate (122.5 mg, 0.66 mmol) at rt and the reaction was stirred at 60° C. for 12 h. The reaction mixture was then cooled to rt and was partitioned between EtOAc (50 mL) and water (10 mL) and the organic phase was concentrated. The residue was purified by column chromatography (SiO₂, EtOAc/pet. ether) to provide tert-butyl 4-(4-((2-(2-((2-(2,6-dioxopiperidin-3-yl)-1,3-dioxoisoindolin-4-yl)amino)ethoxy)ethoxy)methyl)-1H-1,2,3-triazol-1-yl)butanoate (Intermediate 27-1) (0.18 g, 56% yield) as a yellow oil. ¹H NMR (400 MHz, CDCl₃) δ 8.19 (br s, 1H), 7.59 (s, 1H), 7.53-7.43 (m, 1H), 7.11 (d, J=6.8 Hz, 1H), 6.99 (s, 1H), 6.93 (d, J=8.6 Hz, 1H), 4.93 (dd, J=12.0, 5.4 Hz, 1H), 4.75-4.65 (m, 2H), 4.48-4.32 (m, 2H), 3.76-3.67 (m, 6H), 3.49-3.48 (m, 2H), 2.91-2.76 (m, 3H), 2.31-2.15 (m, 5H), 1.45 (s, 9H).

Step 2: A solution of Intermediate 27-1 in 4M HCl in dioxane (20 mL) was stirred at rt for 2 h. The reaction mixture was concentrated to give a residue which was purified by HPLC (85:15 to 65:35 H₂O (0.075% TFA))/CH₃CN) to afford Intermediate 27 (0.08 g, 74% yield) as a yellow oil. ¹H NMR (400 MHz, CDCl₃) δ=8.99 (br s, 1H), 7.62 (s, 1H), 7.50 (t, J=7.8 Hz, 1H), 7.12 (d, J=6.8 Hz, 1H), 6.91 (d, J=8.2 Hz, 1H), 4.98-4.91 (m, 1H), 4.72 (s, 2H), 4.51-4.42 (m, 2H), 3.77-3.67 (m, 6H), 3.47 (br s, 2H), 2.89-2.76 (m, 3H), 2.40-2.14 (m, 5H).

### Example 1

### 4-((R)-3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)-N-(2-(2-(2-((2-(2,6-dioxopiperidin-3-yl)-1,3-dioxoisoindolin-4-yl)amino)ethoxy)ethoxy)ethyl)piperazine-1-carboxamide

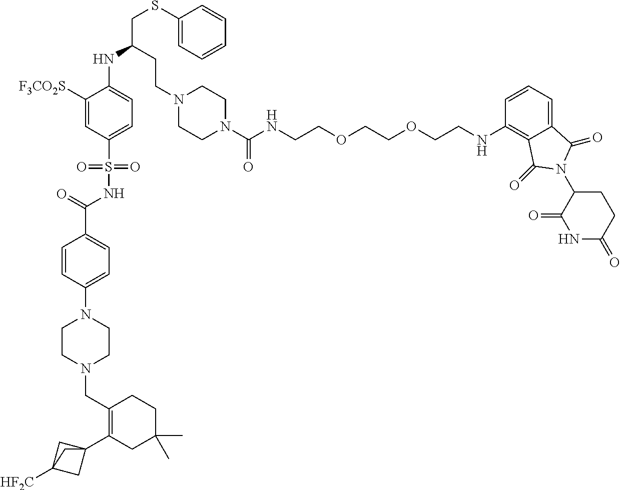

To a stirred solution of Intermediate 17 (153 mg, 0.30 mmol) in DCM (5 mL) at rt was added TEA (85 μL, 0.612 mmol) and carbonyldiimidazole (99.2 mg, 0.612 mmol). After 2 h, Intermediate 12 (100 mg, 0.102 mmol) and DIPEA (7.5 μL, 0.06 mmol) were added and the reaction mixture was stirred at rt for 16 h. The reaction mixture was quenched with sat. aq. NH₄C₁ (5 mL) and extracted with DCM (3×25 mL). The combined organic layers were washed with brine (2×25 mL), dried over Na₂SO₄, filtered and concentrated. The crude product was purified by column chromatography (SiO₂, MeOH/DCM) to afford Example 9 as a yellow solid. ¹H NMR (400 MHz, DMSO-d₆) δ 11.08 (s, 1H), 8.11 (s, 1H), 7.94 (d, J=8.4 Hz 1H), 7.73 (d, J=8.8 Hz, 2H), 7.57 (t, J=7.2 Hz, 1H), 7.38-7.25 (m, 4H), 7.20-7.16 (m, 2H), 7.05-6.75 (m, 4H), 6.60 (t, J=5.6 Hz, 1H), 6.58-6.40 (m, 1H), 6.00 (t, J=56.8 Hz, 1H), 5.06-5.02 (m, 1H), 4.10-4.00 (m, 1H), 3.65-3.42 (m, 12H), 3.35-3.00 (m, 12H), 3.90-2.75 (m, 2H), 2.65-2.53 (m, 4H), 2.50-2.20 (m, 7H), 2.10-1.95 (m, 10H), 1.90-1.70 (m, 3H), 1.35-1.20 (m, 4H), 0.86 (s, 6H); LC/MS (ESI) m/z 1409.4 [M+H]⁺.

### Example 2

### 4-(4-((2-(3-(Difluoromethyl)bicyclo[1.1.1]pentan-1-yl)-4,4-dimethylcyclohex-1-en-1-yl)methyl)piperazin-1-yl)-N-((4-(((2R)-4-(4-(2-(2-((2-(2,6-dioxopiperidin-3-yl)-1,3-dioxoisoindolin-4-yl)amino)ethoxy)ethyl)piperazin-1-yl)-1-(phenylthio)butan-2-yl)amino)-3-((trifluoromethyl)sulfonyl)phenyl)sulfonyl)benzamide

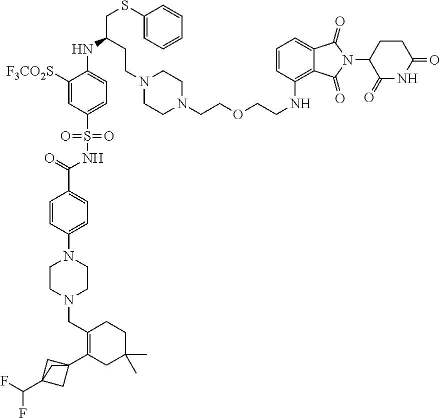

To a stirred solution of Intermediate 12 (220 mg, 0.224 mmol) in 1,4-dioxane (5 mL) was added Intermediate 18 (157.0 mg, 0.359 mmol) followed by NaI (3.0 mg, 0.022 mmol) and DIPEA (0.11 mL, 0.674 mmol) at rt. The reaction mixture was heated to 90° C. and stirred for 2 days. The reaction mixture was then cooled to rt, diluted with ice cold water, and extracted with 10% MeOH in DCM (3×45 mL). The combined organic layers were dried over Na₂SO₄, filtered, and concentrated. The crude product was purified by HPLC (20:80 to 5:95 H₂O (0.05% TFA))/CH₃CN) to afford Example 2 (20 mg, 6% yield) as a yellow solid. ¹H NMR (400 MHz, DMSO-d₆) δ 11.10 (s, 1H), 9.03 (br s, 1H), 8.08 (s, 1H), 7.96 (d, J=8.4 Hz, 1H), 7.72 (d, J=8.8 Hz, 2H), 7.58 (t, J=8.2 Hz, 1H), 7.35-7.25 (m, 4H), 7.20-7.13 (m, 2H), 7.04 (d, J=6.8 Hz, 1H), 6.95-6.65 (m, 4H), 6.63-6.58 (m, 1H), 6.01 (t, J=56.8 Hz, 1H), 5.10-5.00 (m, 1H), 4.01 (br s, 1H), 3.70-3.60 (m, 4H), 3.55-3.45 (m, 2H), 3.33-2.50 (m, 19H), 2.40-1.90 (m, 18H), 1.71 (s, 3H), 1.80-1.70 (m, 2H), 0.86 (s, 6H); LC/MS (ESI) m/z 1322.9 [M+H]⁺.

### Example 3

### (4-(4-((2-(3-(difluoromethyl)bicyclo[1.1.1]pentan-1-yl)-4,4-dimethylcyclohex-1-en-1-yl)methyl)piperazin-1-yl)-N-((4-(((2R)-4-(4-(2-(2-(2-((2-(2,6-dioxopiperidin-3-yl)-1,3-dioxoisoindolin-4-yl)amino)ethoxy)ethoxy)ethyl)piperazin-1-yl)-1-(phenylthio)butan-2-yl)amino)-3-((trifluoromethyl) sulfonyl)phenyl) sulfonyl)benzamide

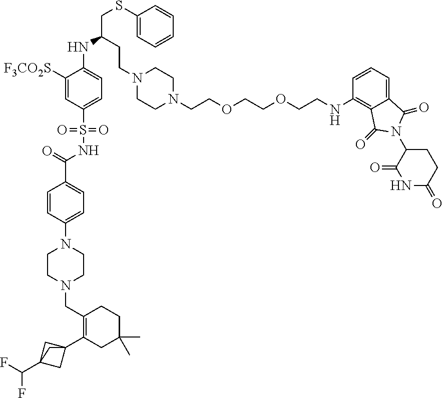

To a stirred solution of Intermediate 12 (130 mg, 0.132 mmol) in DMF (2 mL) was added Intermediate 19 (102 mg, 0.199 mmol) and DIPEA (70 μL, 0.396 mmol) at rt. The reaction was then heated to 40° C. and stirred for 2 days. The reaction mixture was cooled to rt, diluted with water (50 mL) and extracted with 10% MeOH in DCM (3×50 mL). The combined organic layers were washed with 5% NaCl(aq.) (50 mL), dried over Na₂SO₄, filtered and concentrated. The crude product was purified by column chromatography (SiO₂, CH₃CN then MeOH/DCM) followed by HPLC (10 mM NH₄CO₃H(aq.):CH₃CN) to afford Example 3 (16 mg, 6% yield) as a yellow solid. LC/MS (ESI) m/z 1366.3 [M+H]⁺.

### Example 4

### 4-(4-((2-(3-(difluoromethyl)bicyclo[1.1.1]pentan-1-yl)-4,4-dimethylcyclohex-1-en-1-yl)methyl)piperazin-1-yl)-N-((4-(((2R)-4-(4-(2-(2-(2-(2-((2-(2,6-dioxopiperidin-3-yl)-1,3-dioxoisoindolin-4-yl)amino)ethoxy)ethoxy)ethoxy)ethyl)piperazin-1-yl)-1-(phenylthio)butan-2-yl)amino)-3-((trifluoromethyl)sulfonyl)phenyl)sulfonyl)benzamide

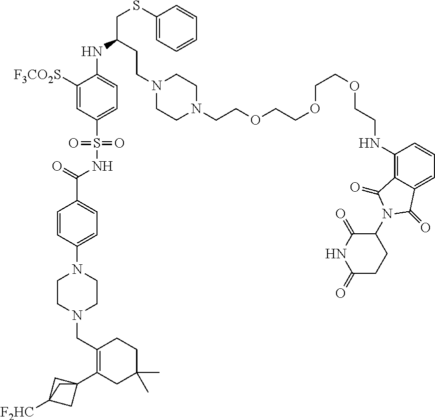

Example 4 was prepared following the procedure described for Example 3 using Intermediate 20 in place of Intermediate 19. ¹H NMR (400 MHz, DMSO-d₆) δ 11.15 (s, 1H), 8.01 (s, 1H), 7.95 (d, J=8.8 Hz, 1H), 7.73 (d, J=8.8 Hz, 2H), 7.59 (t, J=7.2 Hz, 1H), 7.34-7.26 (m, 4H), 7.20-7.17 (m, 1H), 7.14 (d, J=8.8 Hz, 1H), 7.04 (d, J=6.8 Hz, 1H), 6.88-6.81 (m, 3H), 6.70-6.58 (m, 1H), 6.60 (t, J=5.6 Hz, 1H), 6.14 (t, J=56.4 Hz, 1H), 5.08-5.02 (m, 1H), 4.10-4.01 (m, 1H), 3.62-3.46 (m, 14H), 3.46-3.18 (m, 10H), 2.99-2.56 (m, 6H), 2.60-2.32 (m, 12H), 2.09-2.02 (m, 4H), 1.90 (s, 6H), 1.70-1.69 (m, 2H), 1.26-1.22 (m, 2H), 0.86 (s, 6H); LC/MS (ESI) m/z 1410.5 [M+H]⁺.

### Example 5

### 1-((R)-3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)-N-(2-(2-(2-((2-(2,6-dioxopiperidin-3-yl)-1,3-dioxoisoindolin-4-yl)amino)ethoxy)ethoxy)ethyl)piperidine-4-carboxamide

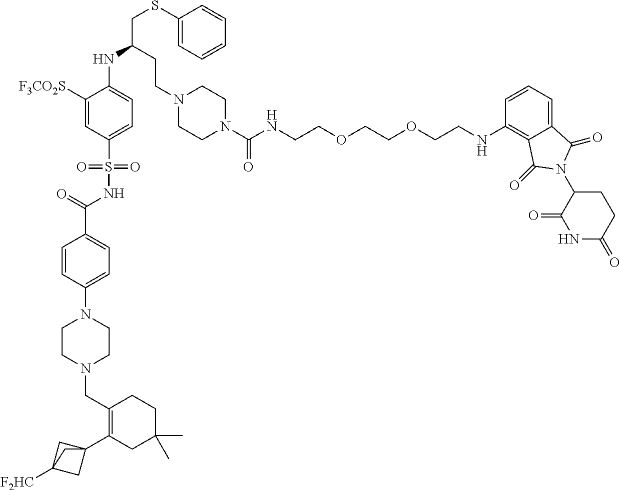

To a stirred solution of Intermediate 22 (120 mg, 0.107 mmol) in DMF (4 mL) at rt was added Intermediate 17 (59.0 mg, 0.117 mmol), HATU (48.9 mg, 0.128 mmol) and DIPEA (0.114 mL, 0.643 mmol). After 16 h, the reaction mixture was quenched with water (10 mL), and extracted with EtOAc (3×10 mL). The combined organic layers were washed with cold water (2×10 mL), brine (10 mL) dried over Na₂SO₄ and concentrated. The crude product was purified by column chromatography (SiO₂, MeOH/DCM) to afford Example 5 as a yellow solid. LC/MS (ESI) m/z 1408.6 [M+H]⁺.

### Example 6

### 4-(4-((2-(3-(Difluoromethyl)bicyclo[1.1.1]pentan-1-yl)-4,4-dimethylcyclohex-1-en-1-yl)methyl)piperazin-1-yl)-N-((4-(((2R)-4-(4-(2-(2-((2-(2,6-dioxopiperidin-3-yl)-1,3-dioxoisoindolin-5-yl)amino)ethoxy)ethyl)piperazin-1-yl)-1-(phenylthio)butan-2-yl)amino)-3-((trifluoromethyl)sulfonyl)phenyl)sulfonyl)benzamide

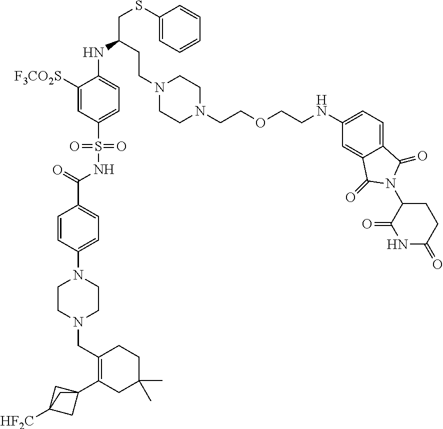

Example 6 was prepared following the procedure described for Example 2 using Intermediate 23 in place of Intermediate 18. LC/MS (ESI) m/z 1322.6 [M+H]⁺.

### Example 7

### 4-(4-((2-(3-(Difluoromethyl)bicyclo[1.1.1]pentan-1-yl)-4,4-dimethylcyclohex-1-en-1-yl)methyl)piperazin-1-yl)-N-((4-(((2R)-4-(4-(2-(2-((2-(2,6-dioxopiperidin-3-yl)-1,3-dioxoisoindolin-4-yl)oxy)ethoxy)ethyl)piperazin-1-yl)-1-(phenylthio)butan-2-yl)amino)-3-((trifluoromethyl)sulfonyl)phenyl)sulfonyl)benzamide

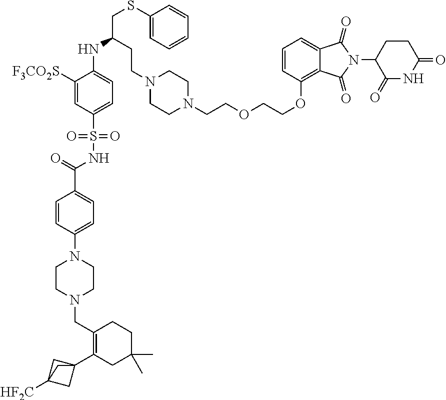

To a stirred solution of Intermediate 12 (150 mg, 0.15 mmol) in 1,4-dioxane (6 mL) was added Intermediate 24 (100.0 mg, 0.22 mmol) followed by DIPEA (0.08 mL, 0.45 mmol) and NaI (2.3 mg, 0.01 mmol) at rt. The reaction mixture was heated to 90° C. and stirred for 2 days. The reaction mixture was then cooled to rt and concentrated. The crude product was diluted with 10% MeOH in DCM (100 mL), washed with water (2×25 mL), brine (2×25 mL), dried over Na₂SO₄, and concentrated. The crude product was purified by HPLC (60:40 to 45:55 10 mM NH₄CO₃H(aq.)/CH₃CN) to afford Example 7 (6.5 mg, 3% yield) as a yellow solid. LC/MS (ESI) m/z 1323.6 [M+H]⁺.

### Example 8

### 4-(4-((2-(3-(Difluoromethyl)bicyclo[1.1.1]pentan-1-yl)-4,4-dimethylcyclohex-1-en-1-yl)methyl)piperazin-1-yl)-N-((4-(((2R)-4-(4-(2-(2-((2-(2,6-dioxopiperidin-3-yl)-1,3-dioxoisoindolin-5-yl)oxy)ethoxy)ethyl)piperazin-1-yl)-1-(phenylthio)butan-2-yl)amino)-3-((trifluoromethyl)sulfonyl)phenyl)sulfonyl)benzamide

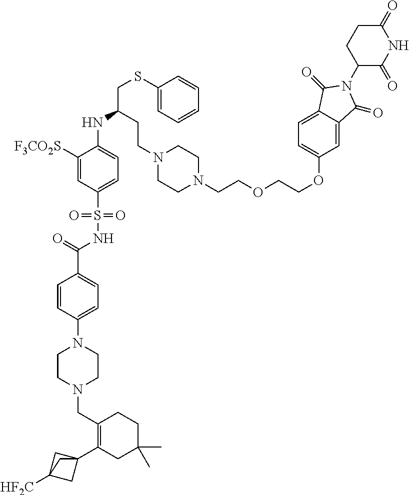

Example 8 was prepared following the procedure described for Example 7 using Intermediate 25 in place of Intermediate 24. ¹H NMR (400 MHz, DMSO-d₆) δ 11.11 (s, 1H, 8.07 (s, 1H), 7.93 (d, J=7.2 Hz, 1H), 7.83 (d, J=8.4 Hz, 1H), 7.72 (d, J=8.8 Hz, 2H), 7.45 (d, J=2.0 Hz, 1H), 7.37-7.26 (m, 5H), 7.20-7.16 (m, 1H), 6.88-6.81 (m, 3H), 6.70 (br s, 1H), 6.00 (t, J=56.4 Hz, 1H), 5.14-5.09 (m, 1H), 4.32 (s, 2H), 4.00 (br s, 1H), 3.79-3.54 (m, 4H), 3.32-3.19 (m, 6H), 3.02-2.85 (m, 5H), 2.67-2.55 (m, 4H), 2.50-2.32 (m, 11H), 2.05-1.99 (m, 6H), 1.99 (s, 6H), 1.70 (s, 3H), 1.26-1.23 (m, 2H), 0.85 (s, 6H); LC/MS (ESI) m/z 1323.6 [M+H]⁺.

### Example 9

### 4-(4-((4,4-Dimethyl-2-(3-methylbicyclo[1.1.1]pentan-1-yl)cyclohex-1-en-1-yl)methyl)piperazin-1-yl)-N-((4-(((2R)-4-(4-(2-(2-((2-(2,6-dioxopiperidin-3-yl)-1,3-dioxoisoindolin-4-yl)amino)ethoxy)ethyl)piperazin-1-yl)-1-(phenylthio)butan-2-yl)amino)-3-((trifluoromethyl)sulfonyl)phenyl)sulfonyl)benzamide

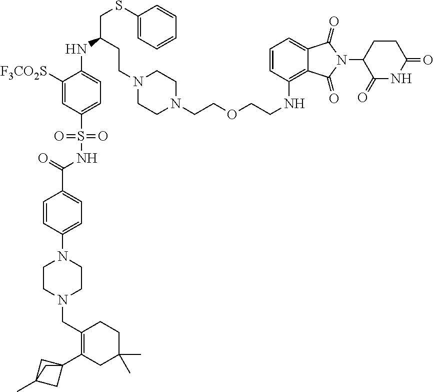

Example 9 was prepared following the procedure described for Example 7 using Intermediate 18 in place of Intermediate 24 and Intermediate 13 in place of Intermediate 12. LC/MS (ESI) m/z 1286.8 [M+H]⁺.

### Example 10

### 4-(4-((4,4-dimethyl-2-(3-methylbicyclo[1.1.1]pentan-1-yl)cyclohex-1-en-1-yl)methyl)piperazin-1-yl)-N-((4-(((2R)-4-(4-(2-(2-(2-(2-((2-(2,6-dioxopiperidin-3-yl)-1,3-dioxoisoindolin-4-yl)amino)ethoxy)ethoxy)ethoxy)ethyl)piperazin-1-yl)-1-(phenylthio)butan-2-yl)amino)-3-((trifluoromethyl)sulfonyl)phenyl)sulfonyl)benzamide

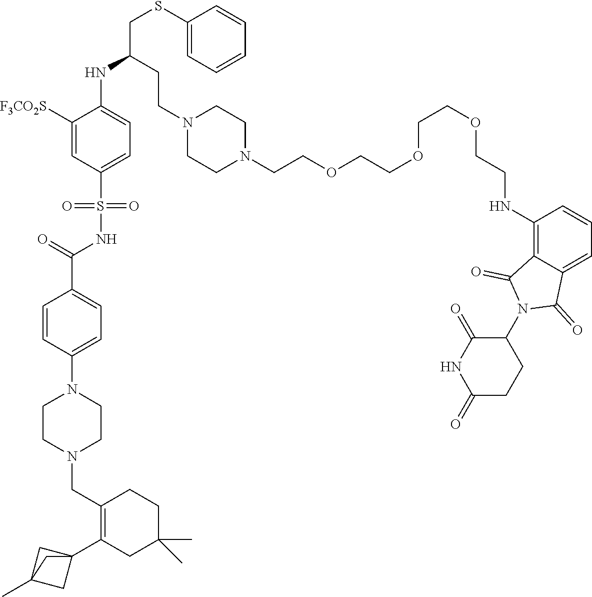

Example 10 was prepared following the procedure described for Example 7 using Intermediate 20-2 in place of Intermediate 24 and Intermediate 13 in place of Intermediate 12. LC/MS (ESI) m/z 1374.9 [M+H]⁺.

### Example 11

### 4-(4-((2-(3-(Difluoromethyl)bicyclo[1.1.1]pentan-1-yl)-4,4-dimethylcyclohex-1-en-1-yl)methyl)piperazin-1-yl)-N-((4-(((2R)-4-((2-(2-(2-((2-(2,6-dioxopiperidin-3-yl)-1,3-dioxoisoindolin-4-yl)amino)ethoxy)ethoxy)ethyl)(methyl)amino)-1-(phenylthio)butan-2-yl)amino)-3-((trifluoromethyl)sulfonyl)phenyl)sulfonyl)benzamide

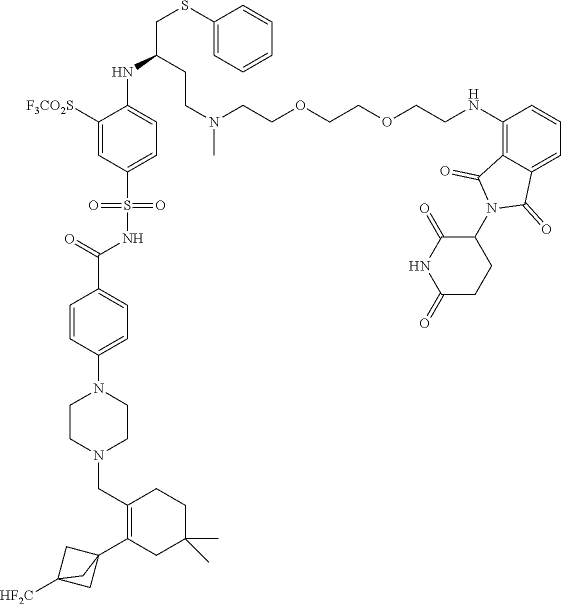

To a stirred solution of Intermediate 26 (120 mg, 0.129 mmol) in 1,4-dioxane (10 mL) was added Intermediate 19 (132 mg, 0.25 mmol), and DIPEA (0.1 mL, 0.51 mmol) at rt. The reaction mixture was heated to 90° C. and stirred for 2 days. The reaction mixture was then cooled to rt and concentrated. The crude product was diluted with 10% MeOH in DCM (50 mL), washed with water (2×15 mL), brine (2×10 mL), dried over Na₂SO₄, and concentrated. The crude product was purified by HPLC (10 mM NH₄CO₃H(aq.)/CH₃CN) to afford Example 11 (13 mg, 8% yield) as a yellow solid. ¹H NMR (400 MHz, DMSO-d₆) δ 11.09 (s, 1H), 8.09 (s, 1H), 7.96 (d, J=5.6 Hz, 1H), 7.72 (d, J=8.8 Hz, 2H), 7.56 (t, J=8.0 Hz, 1H), 7.33 (d, J=7.6 Hz, 2H), 7.27 (t, J=7.2 Hz, 2H), 7.20-7.18 (m, 1H), 7.10 (d, J=8.8 Hz, 1H), 7.03 (d, J=6.8 Hz, 1H), 6.83-6.73 (m, 4H), 6.56 (t, J=5.2 Hz, 1H), 6.00 (t, J=56.4 Hz, 1H), 5.07-5.02 (m, 1H), 4.01 (br s, 1H), 3.56-3.41 (m, 10H), 3.25-3.10 (m, 9H), 2.99-2.88 (br s, 2H), 2.92-2.83 (m, 2H), 2.67-2.55 (m, 5H), 2.50-2.45 (m, 4H), 2.15-2.05 (m, 5H), 1.98 (s, 6H), 1.70 (s, 2H), 1.25 (t, J=6.4 Hz, 2H), 0.86 (s, 6H); LC/MS (ESI) m/z 1311.2 [M+H]⁺.

### Example 12

### 4-(4-((2-(3-(difluoromethyl)bicyclo[1.1.1]pentan-1-yl)-4,4-dimethylcyclohex-1-en-1-yl)methyl)piperazin-1-yl)-N-((4-(((2R)-4-(4-(4-(4-((2-(2-((2-(2,6-dioxopiperidin-3-yl)-1,3-dioxoisoindolin-4-yl)amino)ethoxy)ethoxy)methyl)-1H-1,2,3-triazol-1-yl)butanoyl)piperazin-1-yl)-1-(phenylthio)butan-2-yl)amino)-3-((trifluoromethyl)sulfonyl)phenyl)sulfonyl)benzamide

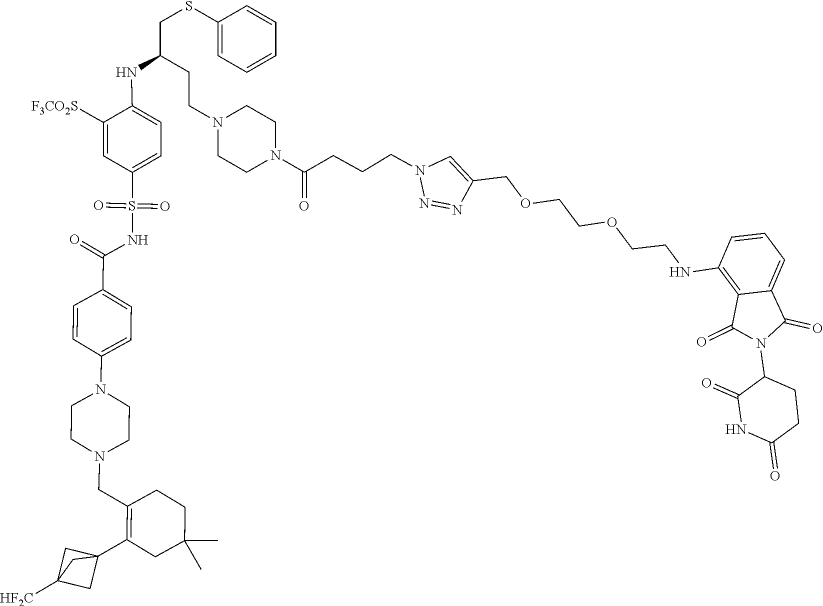

To a solution of Intermediate 12 (166.8 mg, 0.17 mmol), HATU (64.8 mg, 0.17 mmol), and Intermediate 27 (90 mg, 0.17 mmol) in DMF (1 mL) was added DIPEA (88.0 mg, 0.68 mmol) at rt. After 1 hour, the reaction was concentrated and purified by HPLC (53:47 to 0:100 10 mM NH₄CO₃H(aq.)/CH₃CN) to afford Example 12 (35 mg, 14% yield) as a light yellow solid. LC/MS (ESI) m/z 1487.5 (M−H)⁻.

### Example 13

### 4-(4-((2-(3-(difluoromethyl)bicyclo[1.1.1]pentan-1-yl)-4,4-dimethylcyclohex-1-en-1-yl)methyl)piperazin-1-yl)-N-((4-(((2R)-4-(4-(3-(2-(2-((2-(2,6-dioxopiperidin-3-yl)-1,3-dioxoisoindolin-4-yl)amino)ethoxy)ethoxy)propanoyl)piperazin-1-yl)-1-(phenylthio)butan-2-yl)amino)-3-((trifluoromethyl)sulfonyl)phenyl)sulfonyl)benzamide

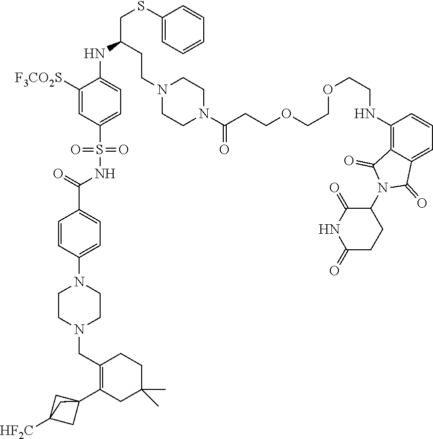

Example 13 was prepared following the procedure described for Example using 3-(2-(2-((2-(2,6-dioxopiperidin-3-yl)-1,3-dioxoisoindolin-4-yl)amino)ethoxy)ethoxy)propanoic acid in place of Intermediate 27. LC/MS (ESI) m/z 1392.4 [M+H]⁺.

### Example 14

### (2S,4R)-1-(2-(3-(2-(2-(4-((R)-3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)ethoxy)ethoxy)propanamido)-3,3-dimethylbutanoyl)-4-hydroxy-N—((S)-1-(4-(4-methylthiazol-5-yl)phenyl)ethyl)pyrrolidine-2-carboxamide

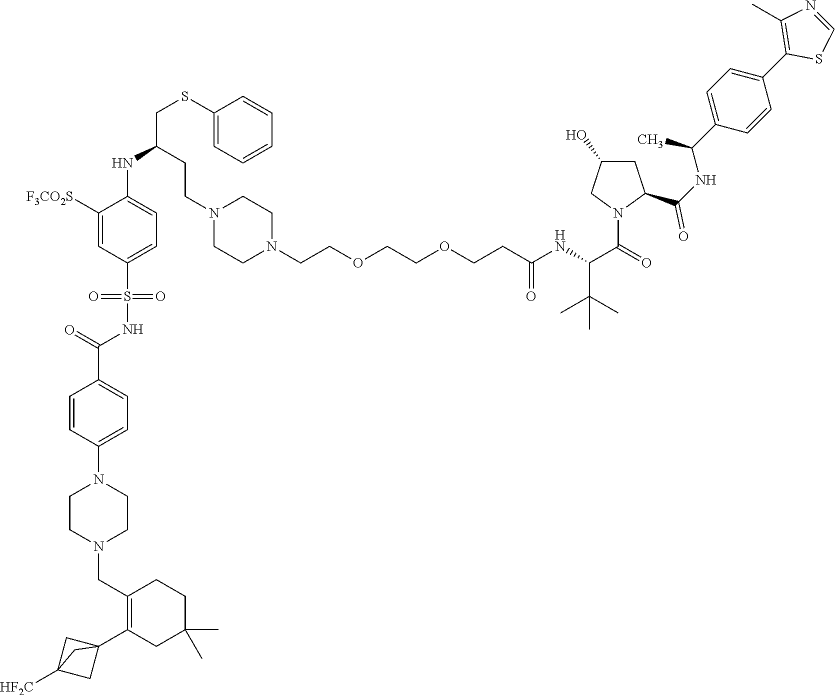

Step 1: To a stirred solution of Intermediate 12 (200 mg, 0.204 mmol) in 1,4-dioxane (5 mL) was added tert-butyl 3-(2-(2-((methylsulfonyl)oxy)ethoxy)ethoxy)propanoate (127 mg, 0.40 mmol) followed by DIPEA (106 μL, 0.61 mmol) and NaI (3 mg, 0.02 mmol) at rt. The reaction mixture was heated to 90° C. and stirred for 24 h. The reaction mixture was cooled to rt, and concentrated. The reaction mixture was dissolved in 10% MeOH in DCM, washed with water (2×10 mL) followed by brine (2×10 mL), dried over Na₂SO₄, filtered and concentrated. The crude product was purified by column chromatography (SiO₂, MeOH/DCM) to afford tert-butyl (R)-3-(2-(2-(4-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperazin-1-yl)ethoxy)ethoxy)propanoate (Example 14-1) (175 mg, 71% yield) as a brown oil. LC/MS (ESI) m/z 1196.1 [M+H]⁺.

Step 2: To a stirred solution of Example 14-1 (200 mg, 0.16 mmol) in dioxane (4 mL) at 0° C., was added 4M HCl in dioxane (2.0 mL). The reaction was warmed to rt and stirred for 16 h. The reaction mixture was concentrated and the residue was diluted with ice cold water, treated with aqueous saturated NaHCO₃ (10 mL) solution and extracted with 10% MeOH in DCM (3×30 mL). The combined organic layers were dried over Na₂SO₄, filtered and concentrated. The crude product was purified by n-pentane and Et₂O triturations to afford (R)-3-(2-(2-(4-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl) sulfonyl)phenyl)amino)-4-(phenylthio)butyl)piperazin-1-yl)ethoxy)ethoxy)propanoic acid (Example 14-2) (165 mg, 86% yield) as an off-white solid. LC/MS (ESI) m/z 1140.0 [M+H]⁺.

Step 3: To a stirred solution of Example 14-2 (150 mg, 0.13 mmol) in DMF (2 mL) was added HATU (82 mg, 0.21 mmol) and DIPEA (75 μL, 0.43 mmol) at 0° C. The reaction was stirred for 30 min at rt and then cooled to 0° C. and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (58.4 mg, 0.13 mmol) was added. The reaction mixture warmed to rt and stirred for 16 h after which it was diluted with 10% MeOH in DCM (75 mL), washed with water (2×15 mL), brine (2×15 mL), dried over Na₂SO₄, filtered and concentrated. The crude product was purified by HPLC (10 mM NH₄CO₃H(aq.)/CH₃CN) to afford Example 14 (15 mg, 0.009 mmol, 7% yield) as an off-white solid. LCMS (ESI) m/z 1565.6 [M+H]⁺.

### Example 15

### N¹-(2-(2-(2-(4-((R)-3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperazine-1-carboxamido)ethoxy)ethoxy)ethyl)-N⁴-(1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)succinamide

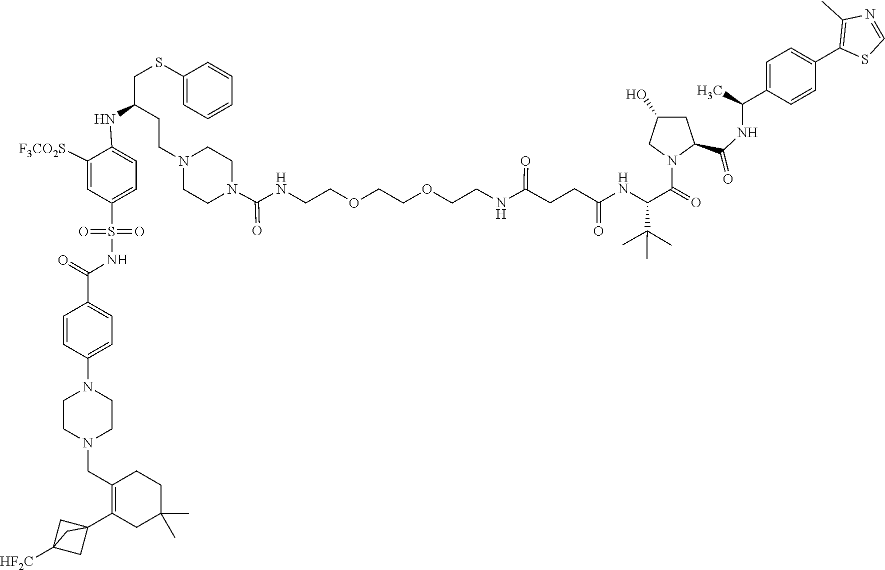

Step 1: To a stirred solution of tert-butyl (2-(2-(2-aminoethoxy)ethoxy)ethyl)carbamate (113 mg, 0.45 mmol) in DCM (6 mL) at 0° C. were added triethylamiine (127 μL, 0.91 mmol) followed by carbonyldiimidazole (99.2 mg, 0.612 mmol). The reaction mixture was warmed rt and stirred for 2 h. The reaction mixture was cooled to 0° C. and treated with a solution of Intermediate 12 (300 mg, 0.30 mmol) in CH₂C₁₂ (3 mL) dropwise. The reaction mixture was warmed to rt. After 16 h, the reaction mixture was diluted with CH₂C₁₂ (30 mL) washed with water (2×10 mL), brine (2×10 mL), dried over Na₂SO₄, filtered and concentrated. The obtained crude was purified by column chromatography (SiO₂, MeOH/DCM) to afford tert-butyl (R)-(2-(2-(2-(4-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl) sulfonyl)phenyl)amino)-4-(phenylthio)butyl)piperazine-1-carboxamido)ethoxy)ethoxy)ethyl)carbamate (Example 15-1) (270 mg, 47% yield) as a colorless oil. LC/MS (ESI) m/z 1253.3 [M+H]⁺.

Step 2: To a stirred solution of Example 15-1 (270 mg, 0.21 mmol) in CH₂C₁₂ (3 mL) was added 4M HCl in 1,4-dioxane (2 mL) at 0° C. The reaction mixture was warmed to rt and stirred for 16 h. The reaction mixture was concentrated and the crude residue was diluted with water, adjusted to ˜pH 8 using sat. aq. NaHCO₃ and extracted with 10% MeOH in CH₂C₁₂ (2×30 mL). The organic layer was dried over Na₂SO₄, filtered and concentrated to afford (R)—N-(2-(2-(2-aminoethoxy)ethoxy)ethyl)-4-(3-((4-(N-(4-(4-((2-(3-(difluoromethyl) bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio) butyl)piperazine-1-carboxamide (Example 15-2) (190 mg, 0.16 mmol, 76%) as a colorless liquid. LC/MS (ESI) m/z 1154.1 [M+H]⁺.

Step 3: To a stirred solution of succinic acid (58 mg, 0.49 mmol) in DMF (4 mL) was added HATU (93 mg, 0.246 mmol) and DIPEA (63 mg, 0.492 mmol). The resulting solution was stirred at rt for 30 min and treated with Example 15-2 (190 mg, 0.16 mmol) at 0° C. and then warmed to rt. After 16 h, the reaction mixture was diluted with 10% MeOH in DCM (30 mL), washed with water (2×20 mL), brine (2×20 mL), dried over Na₂SO₄, filtered and concentrated. The crude product was triturated with CH₂C₁₂ and pentane to afford (R)-1-(4-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl) sulfamoyl)-2-((trifluoromethyl)sulfonyl)phenyl)amino)-4-(phenylthio)butyl)piperazin-1-yl)-1,12-dioxo-5,8-dioxa-2,11-diazapentadecan-15-oic acid (Example 15-3) (150 mg, 73%) as an off-white solid. LC/MS (ESI) m/z 1254.2 [M+H]⁺.

Step 4: To a stirred solution of Example 15-3 (150 mg, 0.11 mmol) in DCM (5 mL) was added EDC.HCl (34 mg, 0.178 mmol), and DMAP (29 mg, 0.238 mmol). The reaction mixture was stirred at rt for 30 min and then cooled to 0° C. and a mixture of (2S,4R)-1-(2-amino-3,3-dimethylbutanoyl)-4-hydroxy-N—((S)-1-(4-(4-methylthiazol-5-yl)phenyl)ethyl)pyrrolidine-2-carboxamide (57 mg, 0.11 mmol) and triethylamine (24 mg, 0.238 mmol) was added. The reaction mixture was then warmed to rt and stirred for 16 h. The reaction mixture was diluted with 10% MeOH in DCM (30 mL), washed with water (2×20 mL), brine (2×20 mL), dried over Na₂SO₄, filtered and concentrated. The crude product was purified by HPLC (70:30 to 35:65 10 mM NH₄CO₃H(aq.)/CH₃CN) to provide Example 15. LC/MS (ESI) m/z 1679.4 [M+H]⁺.

### Example 16

### 4-(4-((4,4-dimethyl-2-(3-methylbicyclo[1.1.1]pentan-1-yl)cyclohex-1-en-1-yl)methyl)piperazin-1-yl)-N-((4-(((2R)-4-(4-(4-(4-((2-(2-((2-(2,6-dioxopiperidin-3-yl)-1,3-dioxoisoindolin-4-yl)amino)ethoxy)ethoxy)methyl)-1H-1,2,3-triazol-1-yl)butanoyl)piperazin-1-yl)-1-(phenylthio)butan-2-yl)amino)-3-((trifluoromethyl)sulfonyl)phenyl)sulfonyl)benzamide

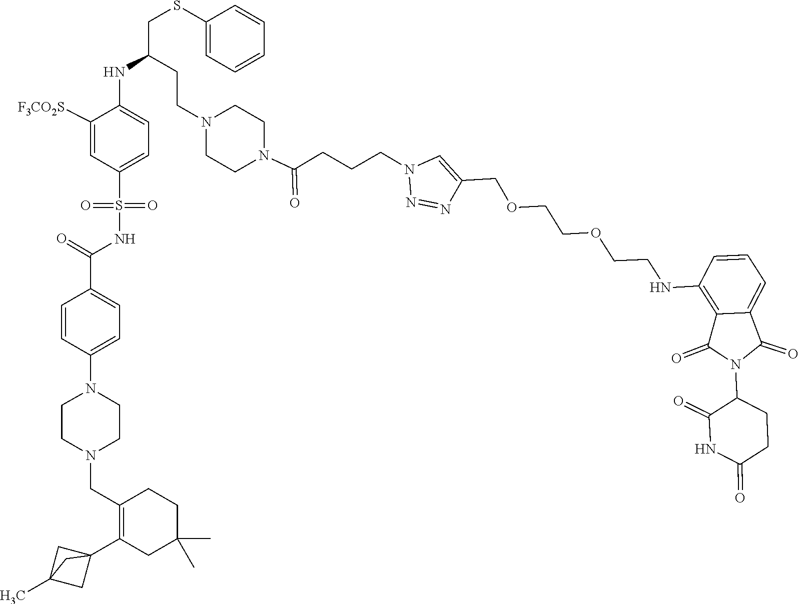

Example 16 is prepared following the procedure described for Example 12 using Intermediate 13 in place of Intermediate 12.

### Example 17

### 4-(4-((4,4-dimethyl-2-(3-methylbicyclo[1.1.1]pentan-1-yl)cyclohex-1-en-1-yl)methyl)piperazin-1-yl)-N-((4-(((2R)-4-(4-(2-(2-(2-(2-((2-(2,6-dioxopiperidin-3-yl)-1,3-dioxoisoindolin-4-yl)amino)ethoxy)ethoxy)ethoxy)ethyl)piperazin-1-yl)-1-(phenylthio)butan-2-yl)amino)-3-((trifluoromethyl)sulfonyl)phenyl)sulfonyl)benzamide

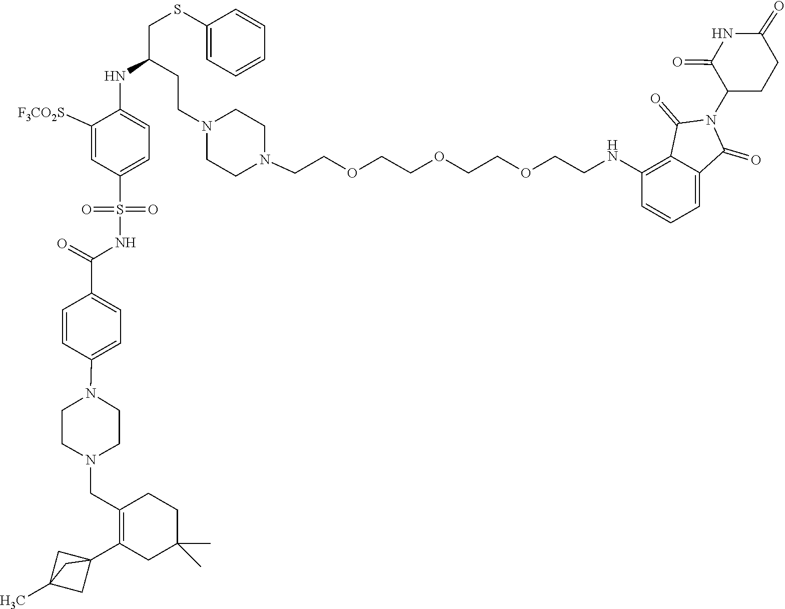

Example 17 is prepared following the procedure described for Example 4 using Intermediate 13 in place of Intermediate 12.

### Example 18

### 4-((R)-3-((4-(N-(4-(4-((4,4-dimethyl-2-(3-methylbicyclo[1.1.1]pentan-1-yl)cyclohex-1-en-1-yl)methyl)piperazin-1-yl)benzoyl) sulfamoyl)-2-((trifluoromethyl)sulfonyl)phenyl)amino)-4-(phenylthio)butyl)-N-(2-(2-(2-((2-(2,6-dioxopiperidin-3-yl)-1,3-dioxoisoindolin-4-yl)amino)ethoxy)ethoxy)ethyl)piperazine-1-carboxamide

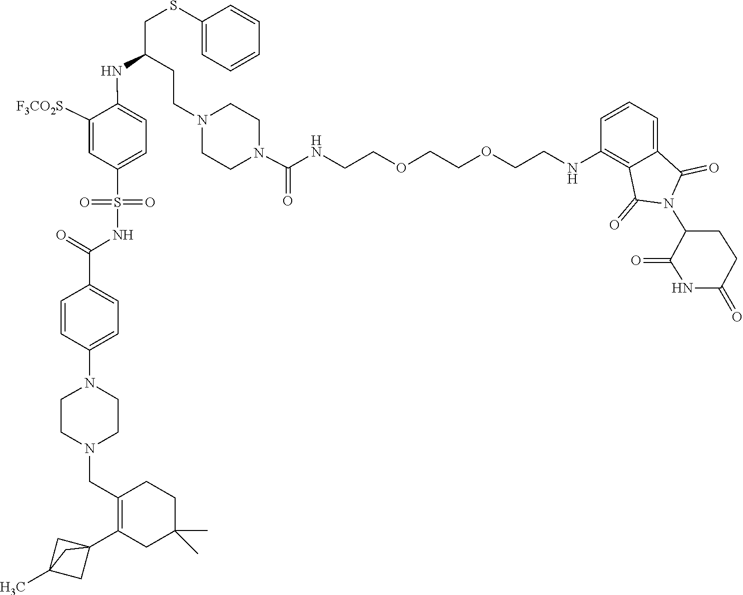

Example 18 is prepared following the procedure described for Example 1 using Intermediate 13 in place of Intermediate 12.

### Example 19

### 1-((R)-3-((4-(N-(4-(4-((4,4-dimethyl-2-(3-methylbicyclo[1.1.1]pentan-1-yl)cyclohex-1-en-1-yl)methyl)piperazin-1-yl)benzoyl) sulfamoyl)-2-((trifluoromethyl)sulfonyl)phenyl)amino)-4-(phenylthio)butyl)piperidin-4-yl (2-(2-(2-((2-(2,6-dioxopiperidin-3-yl)-1,3-dioxoisoindolin-4-yl)amino)ethoxy)ethoxy)ethyl)carbamate

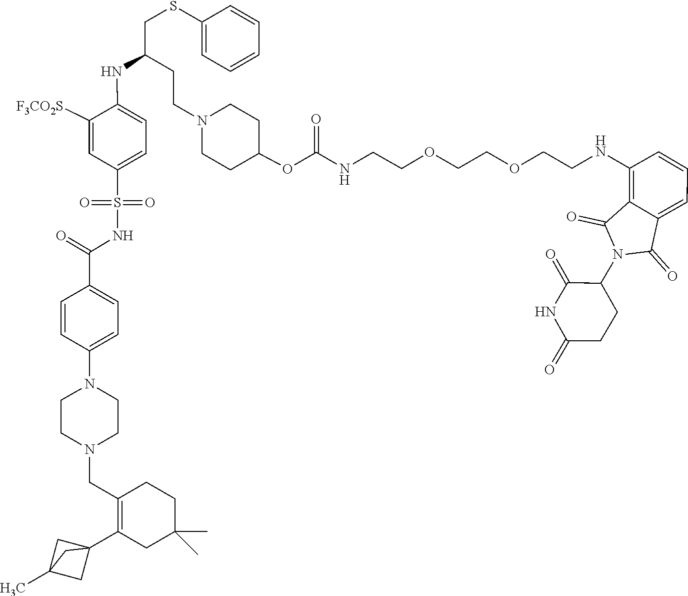

A solution of Intermediate 10 (1.0 eq.), carbonyldiimidazole (1.5 eq.), and DIPEA (3.1 eq.) in 1,2-DCE is heated to 70° C. After the reaction is deemed complete, the reaction is cooled to rt and Intermediate 17 (2.0 eq) in DMSO is added to the reaction mixture. The reaction is then heated to 70° C. After the reaction is deemed complete, the reaction is cooled to rt, quenched with water and extracted with EtOAc. The combined organic layers are washed with brine, and then dried over anhydrous Na₂SO₄, filtered and concentrated. The crude product is purified by column chromatography (SiO₂) to provide Example 19.

### Example 20

### 1-((R)-3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperidin-4-yl (2-(2-(2-((2-(2,6-dioxopiperidin-3-yl)-1,3-dioxoisoindolin-4-yl)amino)ethoxy)ethoxy)ethyl)carbamate

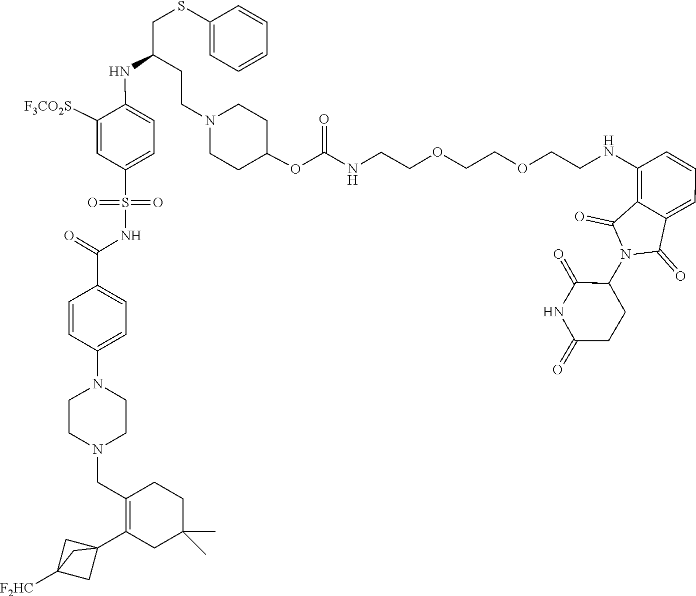

Example 20 is prepared following the procedure described for Example 19 using Intermediate 11 in place of Intermediate 10.

### Example 21

### N-((4-(((2R)-4-(4-(2-(2-(2-(2-((2-(2,6-dioxopiperidin-3-yl)-1,3-dioxoisoindolin-4-yl)amino)ethoxy)ethoxy)ethoxy)ethyl)piperazin-1-yl)-1-(phenylthio)butan-2-yl)amino)-3-((trifluoromethyl)sulfonyl)phenyl)sulfonyl)-4-(4-((2-(3-ethylbicyclo[1.1.1]pentan-1-yl)-4,4-dimethylcyclohex-1-en-1-yl)methyl)piperazin-1-yl)benzamide

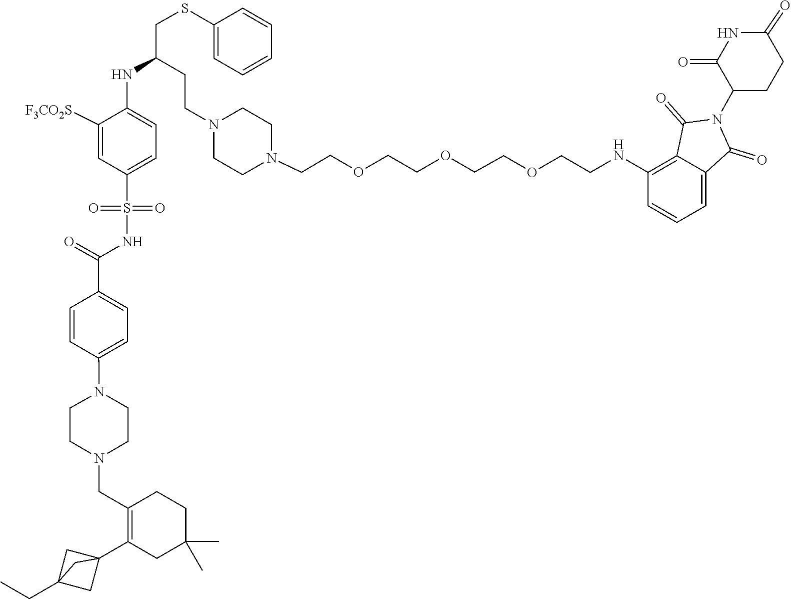

Example 21 is prepared following the procedure described for Example 4 using Intermediate 15 in place of Intermediate 13.

### Example 22

### 1-((R)-3-((4-(N-(4-(4-((2-(3-ethylbicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)piperidin-4-yl (2-(2-(2-((2-(2,6-dioxopiperidin-3-yl)-1,3-dioxoisoindolin-4-yl)amino)ethoxy)ethoxy)ethyl)carbamate

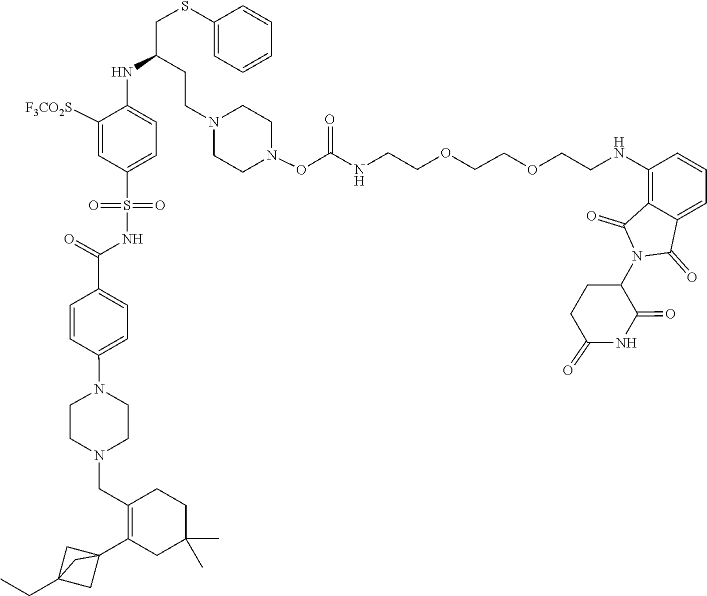

Example 22 is prepared following the procedure described for Example 19 using Intermediate 14 in place of Intermediate 10.

### Example A

### MOLT-4 Cell Proliferation Assay

Cell proliferation was measured using the CellTiter-Glo® Luminescent Cell Viability Assay. The assay involved the addition of a single reagent (CellTiter-Glo® Reagent) directly to cells cultured in serum-supplemented medium. MOLT-4 cells (ATCC, CRL-1582) were cultured according to ATCC recommendations and were seeded at 50,000 cells per well.

Each compound evaluated was prepared as a DMSO stock solution (10 mM). Compounds were tested in duplicate on each plate, with a 10-point serial dilution curve (1:3 dilution). The highest compound concentration was 10 μM (final), with a 0.1% final DMSO concentration. Plates were then incubated at 37° C., 5% CO₂ for 72 h, cell plates were equilibrated at rt for approximately 30 mins. An equi-volume amount of CellTiter-Glo® Reagent (100 μL) was added to each well. Plates were mixed for 2 mins on an orbital shaker to induce cell lysis and then incubated at rt for 10 mins to stabilize the luminescent signal. Luminescence was recorded using a Envision plate reader according to CellTiter-Glo protocol. IC₅₀ of each compound was calculated using GraphPad Prism by nonlinear regression analysis. IC₅₀ values are provided in Table 1.

TABLE 1

| Example# | MOLT-4 (nM) |
|---|---|
| 1 | C |
| 2 | A |
| 4 | A |
| 5 | B |
| 6 | A |
| 9 | A |
| 10 | A |
| ABT-263 | B |

For MOLT-4 CTG IC₅₀: A=a single IC₅₀≤100 nM; B=a single IC₅₀>100 nM and <200 nM; C=a single IC₅₀≥200 nM.

### Example B

### Prow n Degradation Assay in MOLT-4 and MOLM-13 Cells

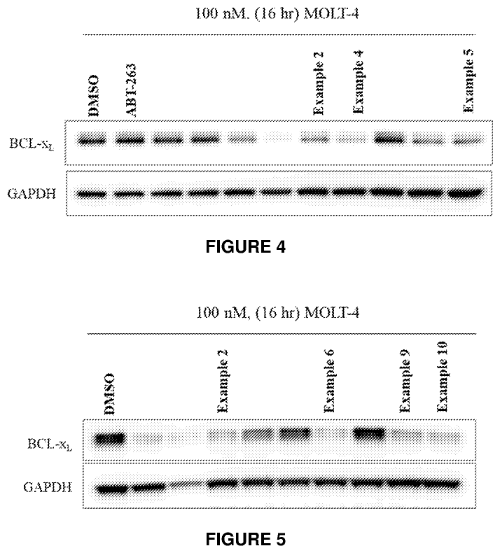

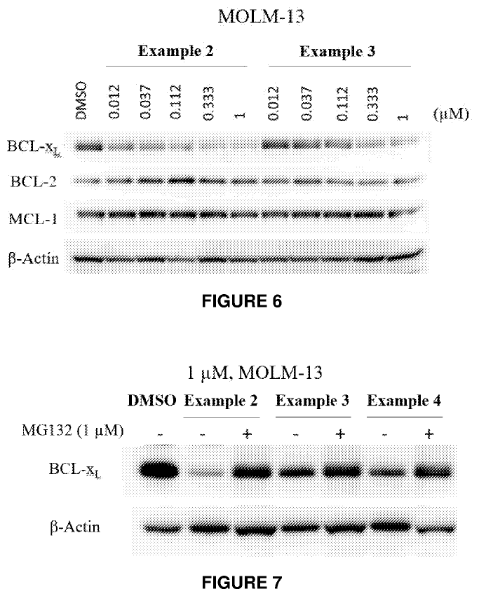

MOLT-4 (ATCC, CRL-1582) (FIGS. 4, 5) were incubated with vehicle or 100 nM concentrations of the indicated compounds for 16 hours. MOLM-13 (DSMZ, ACC554) (FIG. 6,7) cells were incubated with vehicle or increasing concentrations of the indicated compound for 24 hours. For proteasome inhibition, MOLM-13 cells were pretreated with 1 μM of MG132 for 1 hour before the addition of 1 μM of the indicated compounds. After treatment, the cells were harvested in RIPA lysis buffer supplemented with 1% Phosphatase Inhibitor and Protease Inhibitor Cocktail. An equal amount of protein (10 μg/lane) from each cell extract was resolved on a 4-12% Bis-Tris gel. Proteins were transferred using iBlot 2 Transfer Stacks. The membranes were blocked with 5% nonfat milk in TBS-T buffer (50 mM Tris-HCL, pH 7.6; 150 mM NaCl; and 0.05% Tween) and probed with primary antibodies (1:1000 dilution) overnight at 4° C. After three washes with TBS-T (10 min/wash), the membranes were incubated with an appropriate peroxidase-conjugated secondary antibody (Cell Signaling Technology, USA) for 1 hour at rt. After three washes with TBS-T, the proteins of interest were detected with ECL Western Blotting Detection Reagents and captured with an Azure imaging system. The band intensities were determined using ImageJ software and normalized to loading control β-actin or GAPDH. The primary antibodies Bcl-xL (#2762), Bcl-2 (#2872s), Mcl-1 (#5453s) and β-actin (13E5, #4970), and GAPDH (#5174) were purchased from Cell Signaling Technology.

FIGS. 4 and 5 indicate that Examples 2, 4, 5, 6, 9, and 10 induce Bcl-xL degradation in MOLT-4 cells at 100 nM concentrations.

FIG. 6 indicates that Examples 2 and 3 can induce Bcl-xL degradation in MOLM-13 cells in a dose dependent manner.

FIG. 7 indicates that Bcl-xL degradation induced by Examples 2, 3, and 4 can be inhibited by proteasome inhibitor MG132 in MOLM-13 cells.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A compound of Formula (I), or a pharmaceutically acceptable salt thereof, having the structure: 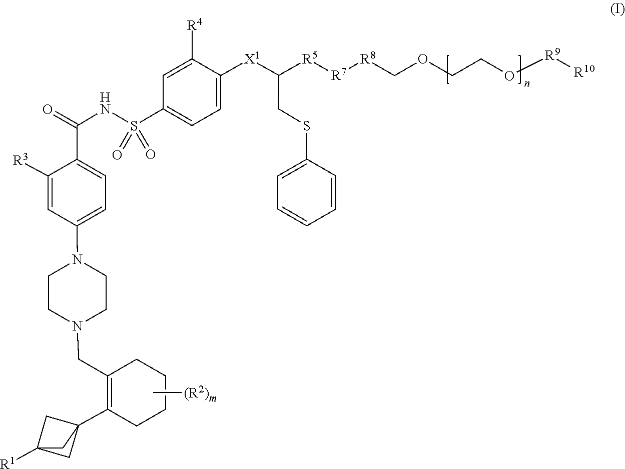
wherein:
R¹ is selected from the group consisting of hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine;
each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl; or
when m is 2 or 3, each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl, or two R² groups taken together with the atom(s) to which they are attached form a substituted or unsubstituted C₃-C₆ cycloalkyl or a substituted or unsubstituted 3 to 6 membered heterocyclyl;
R³ is hydrogen or halogen;
R⁴ is selected from the group consisting of NO₂, S(O)R⁶, SO₂R⁶, halogen, cyano and an unsubstituted C₁-C₆ haloalkyl;
R⁵ is a substituted or unsubstituted C₁-C₆ alkylene, a substituted or unsubstituted —(C₁-C₆ alkylene)-Het-, a substituted or unsubstituted —(C₁-C₆ alkylene)-O—, a substituted or unsubstituted —(C₁-C₆ alkylene)-NH—, a substituted or unsubstituted —(C₁-C₆ alkylene)-Het-O—, a substituted or unsubstituted —(C₁-C₆ alkylene)-Het-NH—, a substituted or unsubstituted —(C₁-C₆ alkylene)-N(C₁-C₆ alkyl)-, a substituted or unsubstituted —(C₁-C₆ alkylene)-Het-N(C₁-C₆ alkyl)-, a substituted or unsubstituted —(C₁-C₆ alkylene)-(C═O)—O— or a substituted or unsubstituted —(C₁-C₆ alkylene)-Het-(C═O)—O—, where Het is a substituted or unsubstituted 3 to 10 membered heterocyclyl;
R⁶ is a substituted or unsubstituted C₁-C₆ alkyl, a substituted or unsubstituted C₁-C₆ haloalkyl or a substituted or unsubstituted C₃-C₆ cycloalkyl;
R⁷ is absent, a substituted or unsubstituted C₁-C₆ alkylene, —(C═O)—, —(C═S)—, —(C═O)—NH—, —(C═O)—O—, —(C═S)—NH—, a substituted or unsubstituted (C₁-C₆ alkylene)-O—, or a substituted or unsubstituted (C₁-C₆ alkylene)-NH—;
R⁸ is absent, a substituted or unsubstituted C₁-C₆ alkylene, a substituted or unsubstituted —(C₁-C₆ alkylene)-(C₆-C₁₂ aryl)-, a substituted or unsubstituted —(C₁-C₆ alkylene)-(C₃-C₁₀ cycloalkyl)-, a substituted or unsubstituted —(C₁-C₆ alkylene)-(C₃-C₁₀ heterocyclyl)-, or a substituted or unsubstituted —(C₁-C₆ alkylene)-(5 to 10 membered heteroaryl)-;
m is 0, 1, 2 or 3;
n is 0, 1, 2, 3, 4 or 5;
X¹ is −O— or —NH—;
R⁹ is a substituted or unsubstituted C₁-C₁₀ alkylene, a substituted or unsubstituted —(C₁-C₆ alkylene)-O—, a substituted or unsubstituted —(C₁-C₆ alkylene)-NH—, a substituted or unsubstituted —(C₁-C₆ alkylene)-NH—(C₁-C₆ alkylene)-, a substituted or unsubstituted —(C₁-C₆ alkylene)-(C═O)NH—, a substituted or unsubstituted —(C₁-C₆ alkylene)-NH—(C₁-C₆ alkylene)-NH—, a substituted or unsubstituted —(C₁-C₆ alkylene)-NH—(C₁-C₆ alkylene)-O—, a substituted or unsubstituted —(C₁-C₆ alkylene)-NH(C═O)—(C₁-C₆ alkylene)-NH—, a substituted or unsubstituted —(C₁-C₆ alkylene)-NH(C═O)—(C₁-C₆ alkylene)-O—, a substituted or unsubstituted —(C₁-C₆ alkylene)-NH(C═O)— (C₁-C₆ alkylene)-, a substituted or unsubstituted —(C₁-C₆ alkylene)-NH— (C₁-C₆ alkylene)-NH(C═O)—(C₁-C₆ alkylene)-NH—, a substituted or unsubstituted —(C₁-C₆ alkylene)-NH—(C₁-C₆ alkylene)-NH(C═O)—(C₁-C₆ alkylene)-O—, a substituted or unsubstituted —(C₁-C₆ alkylene)-NH—(C₁-C₆ alkylene)-NH(C═O)—(C₁-C₆ alkylene)-, a substituted or unsubstituted —(C₁-C₆ alkylene)-(C═O)NH—(C₁-C₆ alkylene)-, a substituted or unsubstituted —(C₁-C₆ alkylene)-(C═O)NH—(C₁-C₆ alkylene)-NH—, a substituted or unsubstituted —(C₁-C₆ alkylene)-(C═O)NH—(C₁-C₆ alkylene)-O—, a substituted or unsubstituted —(C₁-C₆ alkylene)-NH(C═O)—(C₁-C₆ alkylene)-(C═O)NH—, a substituted or unsubstituted —(C₁-C₆ alkylene)-NH—(C₁-C₆ alkylene)-(C═O)NH—, or a substituted or unsubstituted —(C₁-C₆ alkylene)-NH—(C₁-C₆ alkylene)-(C═O)NH—(C₁-C₆ alkylene)-;
R¹⁰ is selected from the group consisting of:

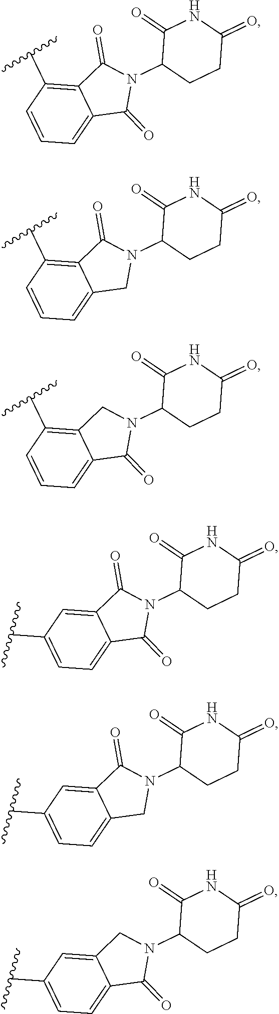 [SMILES: *c1cccc2c1C(=O)N(C1CCC(=O)NC1=O)C2=O ; *c1cccc2c1C(=O)N(C1CCC(=O)NC1=O)C2 ; *c1cccc2c1CN(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O]

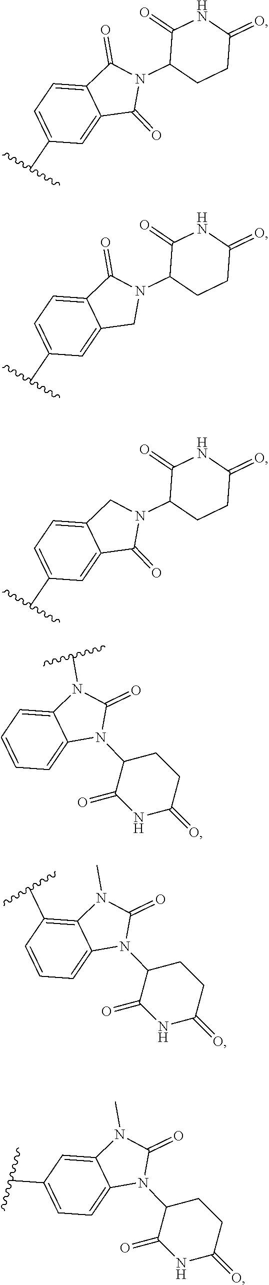 [SMILES: *c1ccc2c(c1)C(=O)N(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *n1c(=O)n(C2CCC(=O)NC2=O)c2ccccc21 ; *c1cccc2c1n(C)c(=O)n2C1CCC(=O)NC1=O ; *c1ccc2c(c1)n(C)c(=O)n2C1CCC(=O)NC1=O]

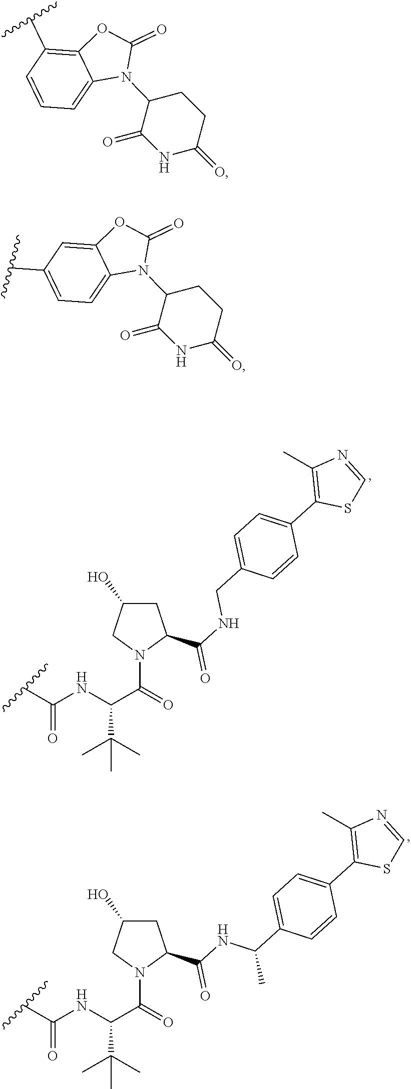 [SMILES: *c1cccc2c1oc(=O)n2C1CCC(=O)NC1=O ; *c1ccc2c(c1)oc(=O)n2C1CCC(=O)NC1=O ; *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C]

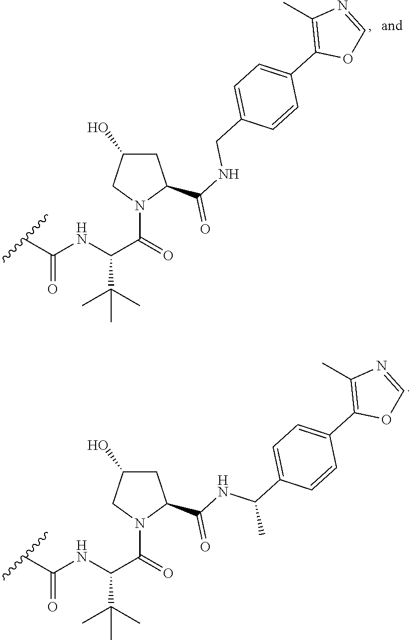 [SMILES: *C(=O)N[C@H](C(=O)N1C[C@H](O)C[C@H]1C(=O)NCc1ccc(-c2ocnc2C)cc1)C(C)(C)C ; *C(=O)N[C@H](C(=O)N1C[C@H](O)C[C@H]1C(=O)N[C@@H](C)c1ccc(-c2ocnc2C)cc1)C(C)(C)C]

2. The compound of claim 1, or a pharmaceutically acceptable salt thereof, wherein R¹ is halogen.

3. The compound of any one of claims 1-2, or a pharmaceutically acceptable salt thereof, wherein R¹ is fluoro.

4. The compound of any one of claims 1-2, or a pharmaceutically acceptable salt thereof, wherein R¹ is chloro.

5. The compound of claim 1, or a pharmaceutically acceptable salt thereof, wherein R¹ is a substituted or unsubstituted C₁-C₆ alkyl.

6. The compound of claim 1 or 5, or a pharmaceutically acceptable salt thereof, wherein R¹ is an unsubstituted C₁-C₆ alkyl.

7. The compound of any one of claim 1 or 5-6, or a pharmaceutically acceptable salt thereof, wherein R¹ is an unsubstituted methyl or an unsubstituted ethyl.

8. The compound of claim 1, or a pharmaceutically acceptable salt thereof, wherein R¹ is a substituted or unsubstituted C₁-C₆ haloalkyl.

9. The compound of claim 1 or 8, or a pharmaceutically acceptable salt thereof, wherein R¹ is an unsubstituted —CHF₂, —CF₃, —CH₂CF₃, —CF₂CF₃ or —CF₂CH₃.

10. The compound of claim 1, or a pharmaceutically acceptable salt thereof, wherein R¹ is hydrogen.

11. The compound of claim 1, or a pharmaceutically acceptable salt thereof, wherein R¹ is a substituted or unsubstituted C₃-C₆ cycloalkyl.

12. The compound of claim 1, or a pharmaceutically acceptable salt thereof, wherein R¹ is an unsubstituted C₃-C₆ cycloalkyl.

13. The compound of claim 1, or a pharmaceutically acceptable salt thereof, wherein R¹ is a substituted or unsubstituted C₁-C₆ alkoxy.

14. The compound of claim 1 or 13, or a pharmaceutically acceptable salt thereof, wherein R¹ is an unsubstituted C₁-C₆ alkoxy.

15. The compound of any one of claim 1 or 13-14, or a pharmaceutically acceptable salt thereof, wherein R¹ is an unsubstituted methoxy or an unsubstituted ethoxy.

16. The compound of claim 1, or a pharmaceutically acceptable salt thereof, wherein R¹ is an unsubstituted mono-C₁-C₆ alkylamine.

17. The compound of claim 1 or 16, or a pharmaceutically acceptable salt thereof, wherein R¹ is methylamine or ethylamine.

18. The compound of claim 1, or a pharmaceutically acceptable salt thereof, wherein R¹ is an unsubstituted di-C₁-C₆ alkylamine.

19. The compound of claim 1 or 18, or a pharmaceutically acceptable salt thereof, wherein R¹ is di-methylamine or di-ethylamine.

20. The compound of any one of claims 1-19, or a pharmaceutically acceptable salt thereof, wherein m is 1.

21. The compound of any one of claims 1-19, or a pharmaceutically acceptable salt thereof, wherein m is 2.

22. The compound of any one of claims 1-19, or a pharmaceutically acceptable salt thereof, wherein m is 3.

23. The compound of any one of claims 1-22, or a pharmaceutically acceptable salt thereof, wherein one R² is an unsubstituted C₁-C₆ alkyl and any other R², if present, is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl.

24. The compound of any one of claims 1-22, or a pharmaceutically acceptable salt thereof, wherein each R² is independently an unsubstituted C₁-C₆ alkyl.

25. The compound of any one of claim 1-19, 23 or 24, or a pharmaceutically acceptable salt thereof, wherein m is 2; and each R² is an unsubstituted methyl.

26. The compound of any one of claims 1-19, or a pharmaceutically acceptable salt thereof, wherein m is 0.

27. The compound of any one of claim 1-19 or 21-22, or a pharmaceutically acceptable salt thereof, wherein two R² groups taken together with the atom(s) to which they are attached form a substituted or unsubstituted C₃-C₆ cycloalkyl.

28. The compound of any one of claim 1-19, 21-22 or 27, or a pharmaceutically acceptable salt thereof, wherein two R² groups taken together with the atom to which they are attached form an unsubstituted cyclopropyl.

29. The compound of any one of claim 1-19, 21-22 or 27, or a pharmaceutically acceptable salt thereof, wherein two R² groups taken together with the atom to which they are attached form an unsubstituted cyclobutyl.

30. The compound of any one of claim 1-19 or 21-22, or a pharmaceutically acceptable salt thereof, wherein two R² groups taken together with the atom(s) to which they are attached form a substituted or unsubstituted 3 to 6 membered heterocyclyl.

31. The compound of any one of claims 1-30, wherein R³ is hydrogen.

32. The compound of any one of claims 1-30, wherein R³ is halogen.

33. The compound of any one of claims 1-32, or a pharmaceutically acceptable salt thereof, wherein R⁴ is NO₂.

34. The compound of any one of claims 1-32, or a pharmaceutically acceptable salt thereof, wherein R⁴ is cyano.

35. The compound of any one of claims 1-32, or a pharmaceutically acceptable salt thereof, wherein R⁴ is halogen.

36. The compound of any one of claims 1-32, or a pharmaceutically acceptable salt thereof, wherein R⁴ is an unsubstituted C₁-C₆ haloalkyl.

37. The compound of any one of claim 1-32 or 36, or a pharmaceutically acceptable salt thereof, wherein R⁴ is —CF₃.

38. The compound of any one of claims 1-32, or a pharmaceutically acceptable salt thereof, wherein R⁴ is S(O)R⁶.

39. The compound of any one of claims 1-32, or a pharmaceutically acceptable salt thereof, wherein R⁴ is SO₂R⁶.

40. The compound of any one of claim 1-32 or 38-39, or a pharmaceutically acceptable salt thereof, wherein R⁶ is a substituted or unsubstituted C₁-C₆ alkyl.

41. The compound of any one of claim 1-32 or 38-39, or a pharmaceutically acceptable salt thereof, wherein R⁶ is a substituted or unsubstituted C₃-C₆ cycloalkyl.

42. The compound of any one of claim 1-32 or 38-39, or a pharmaceutically acceptable salt thereof, wherein R⁶ is a substituted or unsubstituted C₁-C₆ haloalkyl.

43. The compound of any one of claim 38-39 or 42, or a pharmaceutically acceptable salt thereof, wherein R⁶ is —CF₃.

44. The compound of any one of claims 1-43, or a pharmaceutically acceptable salt thereof, wherein R⁵ is a substituted or unsubstituted C₁-C₆ alkylene, a substituted or unsubstituted —(C₁-C₆ alkylene)-O—, a substituted or unsubstituted —(C₁-C₆ alkylene)-NH—, or a substituted or unsubstituted —(C₁-C₆ alkylene)-N(C₁-C₆ alkyl)-.

45. The compound of any one of claims 1-43, or a pharmaceutically acceptable salt thereof, wherein R⁵ is a substituted or unsubstituted —(C₁-C₆ alkylene)-Het-, a substituted or unsubstituted —(C₁-C₆ alkylene)-Het-O—, a substituted or unsubstituted —(C₁-C₆ alkylene)-Het-NH—, a substituted or unsubstituted —(C₁-C₆ alkylene)-Het-N(C₁-C₆ alkyl)-, a substituted or unsubstituted —(C₁-C₆ alkylene)-(C═O)—O—, or a substituted or unsubstituted —(C₁-C₆ alkylene)-Het-(C═O)—O—.

46. The compound of claim 45, or a pharmaceutically acceptable salt thereof, wherein Het is a substituted or unsubstituted azetidinyl, pyrrolidinyl, piperidinyl, or piperazinyl.

47. The compound of any one of claims 1-46, or a pharmaceutically acceptable salt thereof, wherein X¹ is −O—.

48. The compound of any one of claims 1-46, or a pharmaceutically acceptable salt thereof, wherein X¹ is —NH—.

49. The compound of any one of claims 1-48, or a pharmaceutically acceptable salt thereof, wherein R⁷ is absent.

50. The compound of any one of claims 1-48, or a pharmaceutically acceptable salt thereof, wherein R⁷ is a substituted or unsubstituted C₁-C₆ alkylene.

51. The compound of any one of claims 1-48, or a pharmaceutically acceptable salt thereof, wherein R⁷ is —(C═O)—, —(C═S)—, —(C═O)—NH—, —(C═O)—O—, or —(C═S)—NH—.

52. The compound of any one of claims 1-48, or a pharmaceutically acceptable salt thereof, wherein R⁷ is a substituted or unsubstituted —(C₁-C₆ alkylene)-O— or a substituted or unsubstituted —(C₁-C₆ alkylene)-NH—.

53. The compound of any one of claims 1-48, or a pharmaceutically acceptable salt thereof, wherein R⁵ and R⁷ are selected such that —R⁵-R⁷— is selected from:

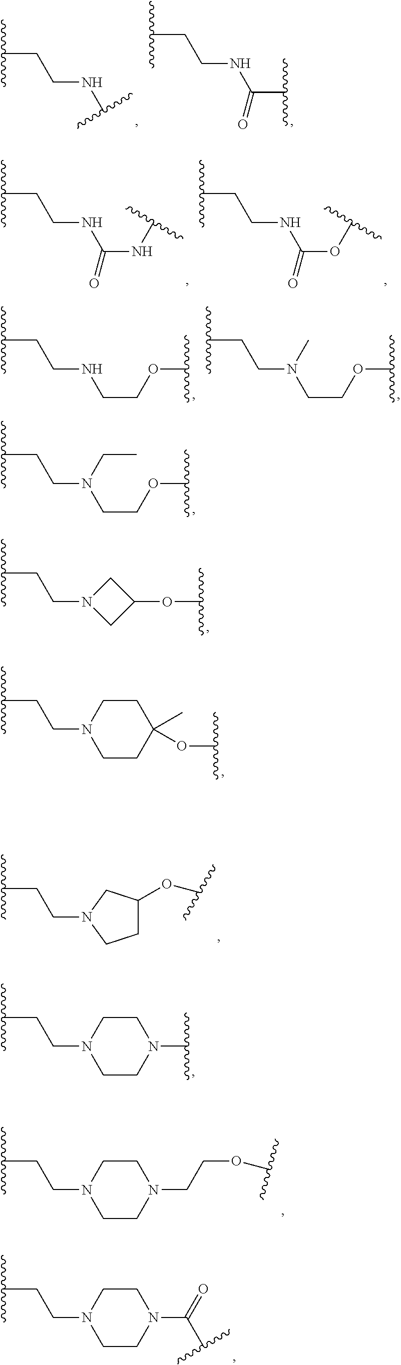

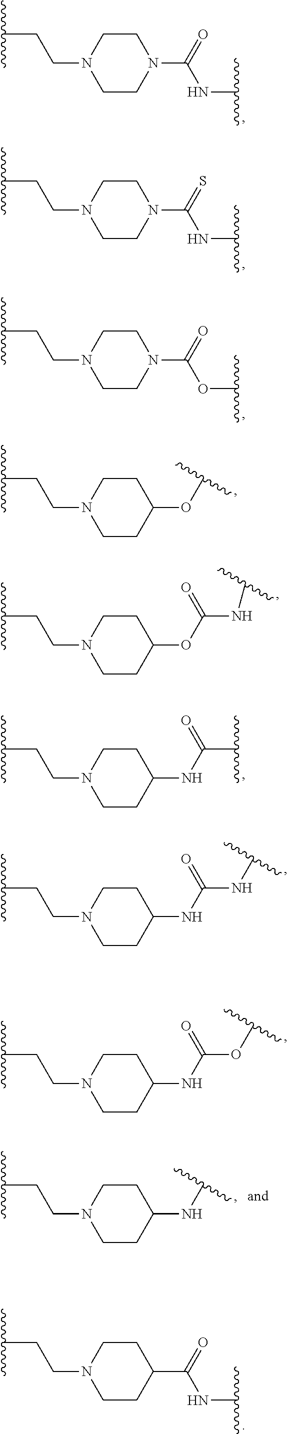

54. The compound of any one of claims 1-53, or a pharmaceutically acceptable salt thereof, wherein R⁸ is absent.

55. The compound of any one of claims 1-53, or a pharmaceutically acceptable salt thereof, wherein R⁸ is a substituted or unsubstituted C₁-C₆ alkylene.

56. The compound of any one of claims 1-53, or a pharmaceutically acceptable salt thereof, wherein R⁸ is a substituted or unsubstituted —(C₁-C₆ alkylene)-(C₆-C₁₂ aryl)-, a substituted or unsubstituted —(C₁-C₆ alkylene)-(C₃-C₁₀ cycloalkyl)-, a substituted or unsubstituted —(C₁-C₆ alkylene)-(C₃-C₁₀ heterocyclyl)-, or a substituted or unsubstituted —(C₁-C₆ alkylene)-(5 to 10 membered heteroaryl)-.

57. The compound of any one of claims 1-56, or a pharmaceutically acceptable salt thereof, wherein n is 1, 2, 3, 4 or 5.

58. The compound of any one of claims 1-56, or a pharmaceutically acceptable salt thereof, wherein n is 0.

59. The compound of any one of claims 1-58, or a pharmaceutically acceptable salt thereof, wherein R⁹ is a substituted or unsubstituted C₁-C₁₀ alkylene, a substituted or unsubstituted —(C₁-C₆ alkylene)-O—, a substituted or unsubstituted —(C₁-C₆ alkylene)-NH—, a substituted or unsubstituted —(C₁-C₆ alkylene)-NH—(C₁-C₆ alkylene)-, or a substituted or unsubstituted —(C₁-C₆ alkylene)-(C═O)NH—.

60. The compound of any one of claims 1-58, or a pharmaceutically acceptable salt thereof, wherein R⁹ is a substituted or unsubstituted —(C₁-C₆ alkylene)-NH—(C₁-C₆ alkylene)-NH—, a substituted or unsubstituted —(C₁-C₆ alkylene)-NH—(C₁-C₆ alkylene)-O—, a substituted or unsubstituted —(C₁-C₆ alkylene)-NH(C═O)—(C₁-C₆ alkylene)-NH—, a substituted or unsubstituted —(C₁-C₆ alkylene)-NH(C═O)—(C₁-C₆ alkylene)-O—, or a substituted or unsubstituted —(C₁-C₆ alkylene)-NH(C═O)—(C₁-C₆ alkylene)-.

61. The compound of any one of claims 1-58, or a pharmaceutically acceptable salt thereof, wherein R⁹ is a substituted or unsubstituted —(C₁-C₆ alkylene)-NH—(C₁-C₆ alkylene)-NH(C═O)—(C₁-C₆ alkylene)-NH—, a substituted or unsubstituted —(C₁-C₆ alkylene)-NH—(C₁-C₆ alkylene)-NH(C═O)—(C₁-C₆ alkylene)-O—, a substituted or unsubstituted —(C₁-C₆ alkylene)-NH—(C₁-C₆ alkylene)-NH(C═O)—(C₁-C₆ alkylene)-, a substituted or unsubstituted —(C₁-C₆ alkylene)-(C═O)NH—(C₁-C₆ alkylene)-, a substituted or unsubstituted —(C₁-C₆ alkylene)-(C═O)NH—(C₁-C₆ alkylene)-NH—, a substituted or unsubstituted —(C₁-C₆ alkylene)-(C═O)NH—(C₁-C₆ alkylene)-O—, a substituted or unsubstituted —(C₁-C₆ alkylene)-NH(C═O)—(C₁-C₆ alkylene)-(C═O)NH—, a substituted or unsubstituted —(C₁-C₆ alkylene)-NH—(C₁-C₆ alkylene)-(C═O)NH—, or a substituted or unsubstituted —(C₁-C₆ alkylene)-NH—(C₁-C₆ alkylene)-(C═O)NH—(C₁-C₆ alkylene)-.

62. The compound of any one of claims 1-61, or a pharmaceutically acceptable salt thereof, wherein R¹⁰ is selected from the group consisting of:

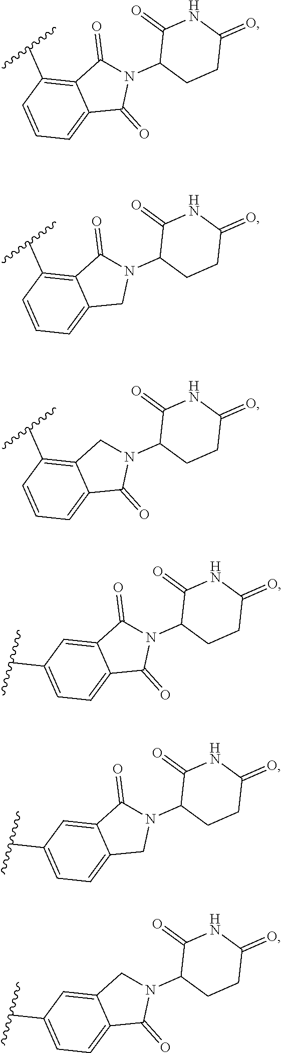 [SMILES: *c1cccc2c1C(=O)N(C1CCC(=O)NC1=O)C2=O ; *c1cccc2c1C(=O)N(C1CCC(=O)NC1=O)C2 ; *c1cccc2c1CN(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O]

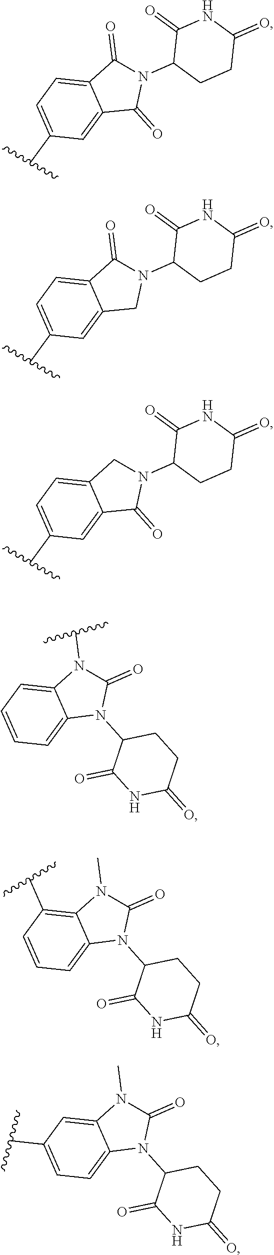 [SMILES: *c1ccc2c(c1)C(=O)N(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *n1c(=O)n(C2CCC(=O)NC2=O)c2ccccc21 ; *c1cccc2c1n(C)c(=O)n2C1CCC(=O)NC1=O ; *c1ccc2c(c1)n(C)c(=O)n2C1CCC(=O)NC1=O]

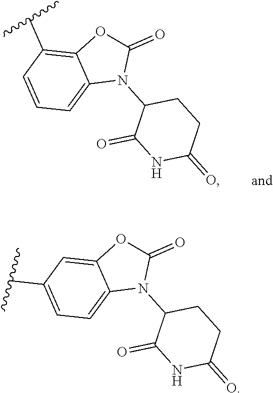 [SMILES: *c1cccc2c1oc(=O)n2C1CCC(=O)NC1=O ; "and" ; *c1ccc2c(c1)oc(=O)n2C1CCC(=O)NC1=O]

63. The compound of any one of claims 1-60, or a pharmaceutically acceptable salt thereof, wherein R¹⁰ is selected from the group consisting of:

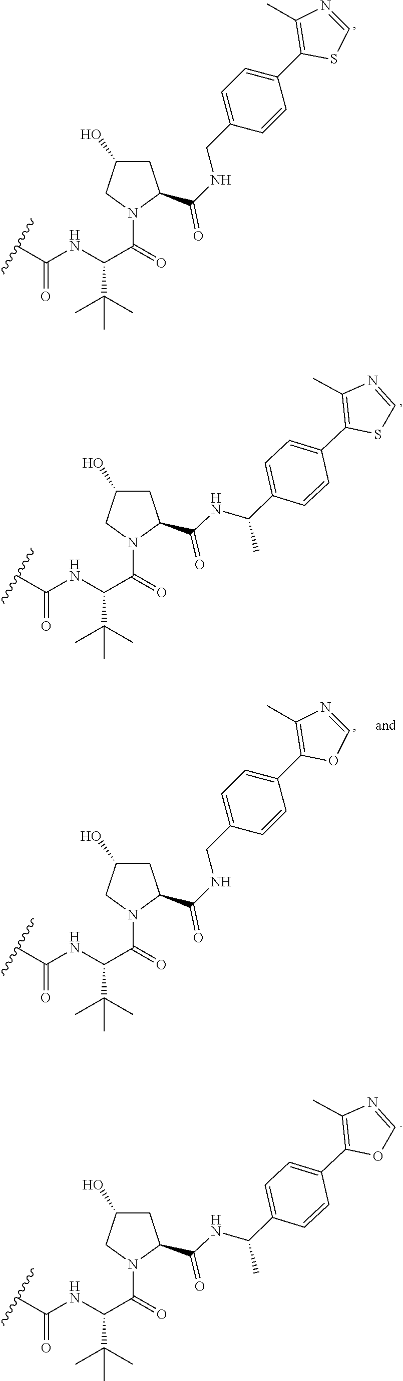 [SMILES: *C(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C ; *C(=O)N[C@H](C(=O)N1C[C@H](O)C[C@H]1C(=O)NCc1ccc(-c2ocnc2C)cc1)C(C)(C)C ; *C(=O)N[C@H](C(=O)N1C[C@H](O)C[C@H]1C(=O)N[C@@H](C)c1ccc(-c2ocnc2C)cc1)C(C)(C)C]

64. The compound of claim 1, wherein the compound is selected from the group consisting of: 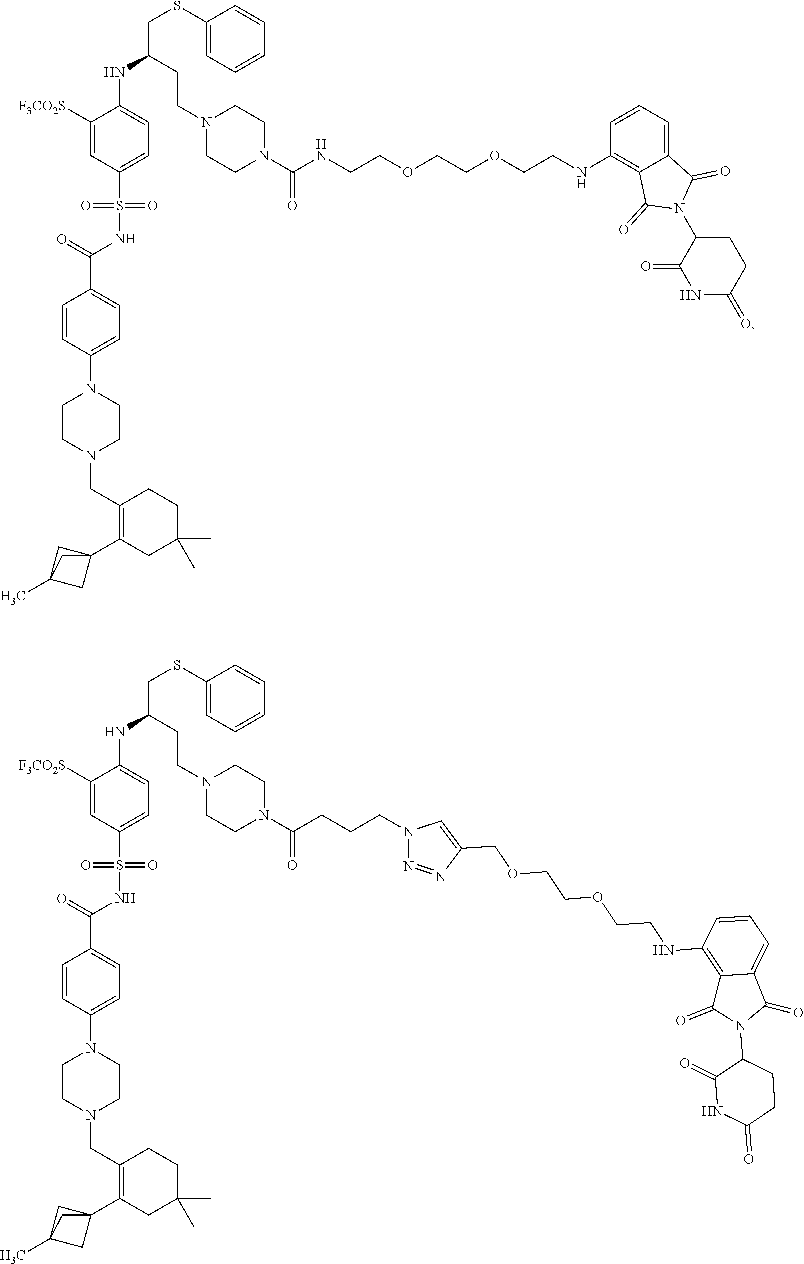 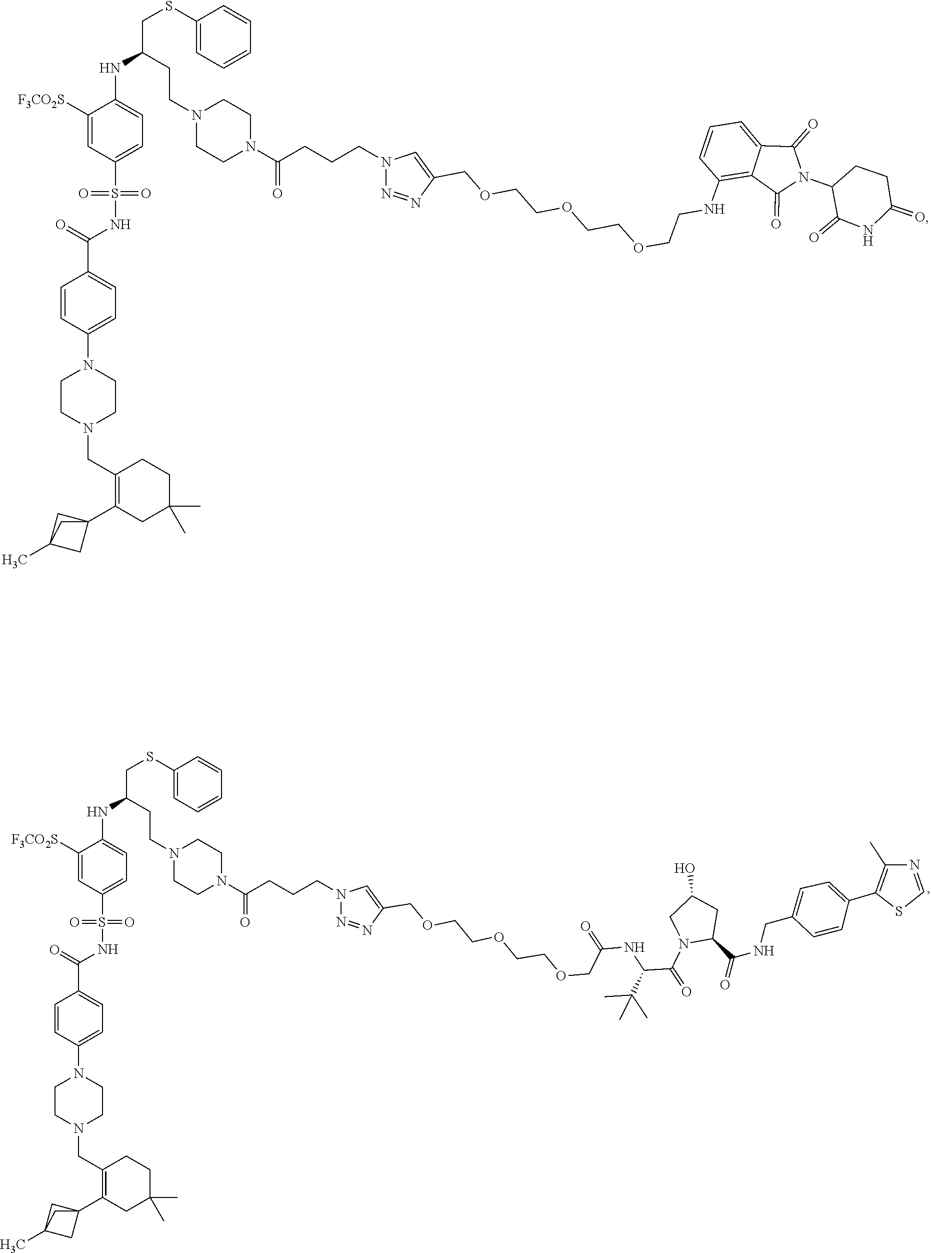 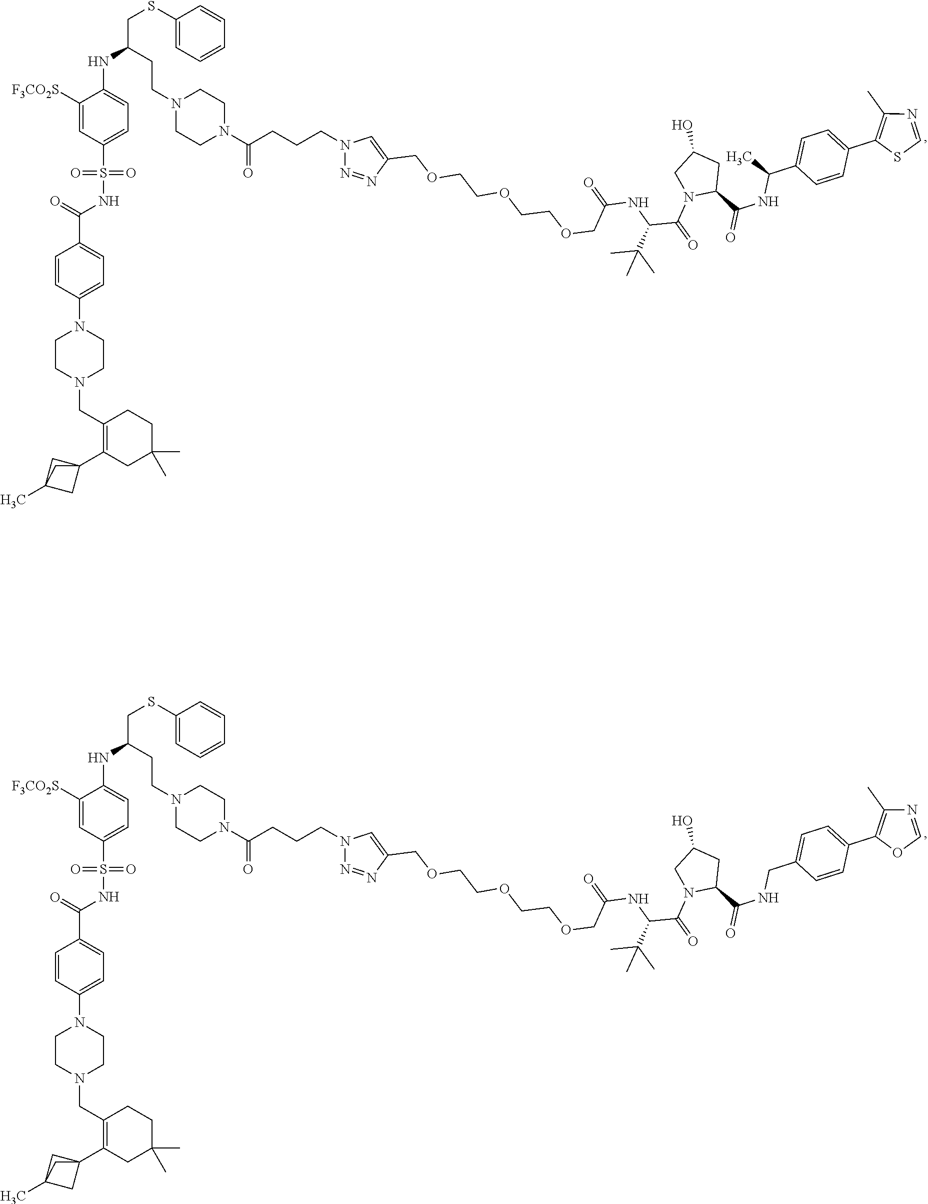 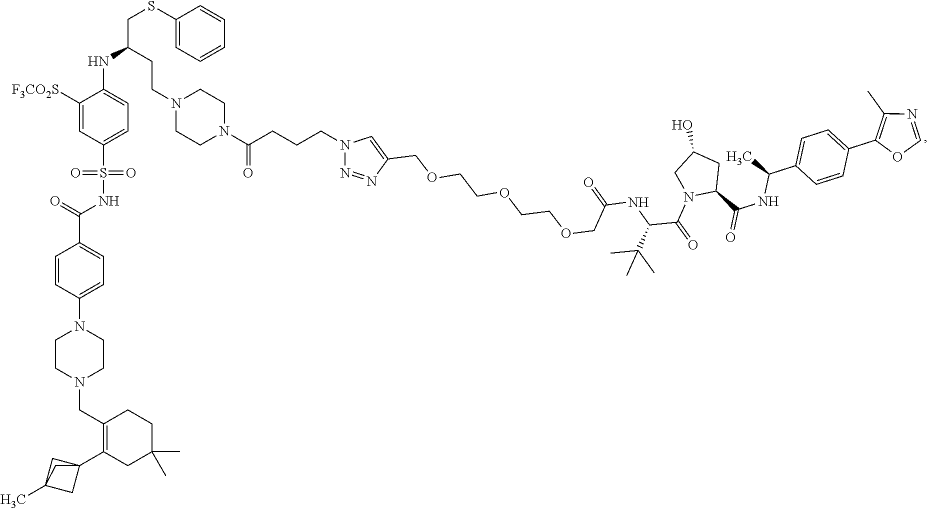 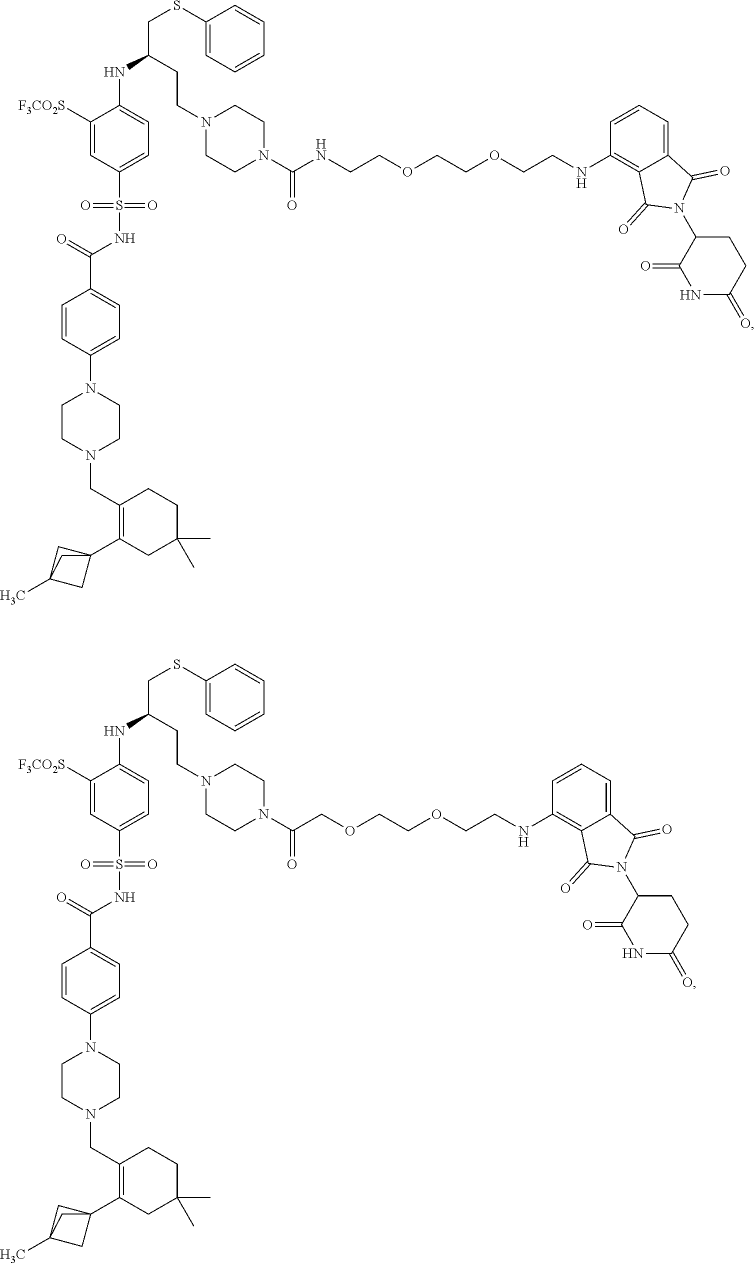 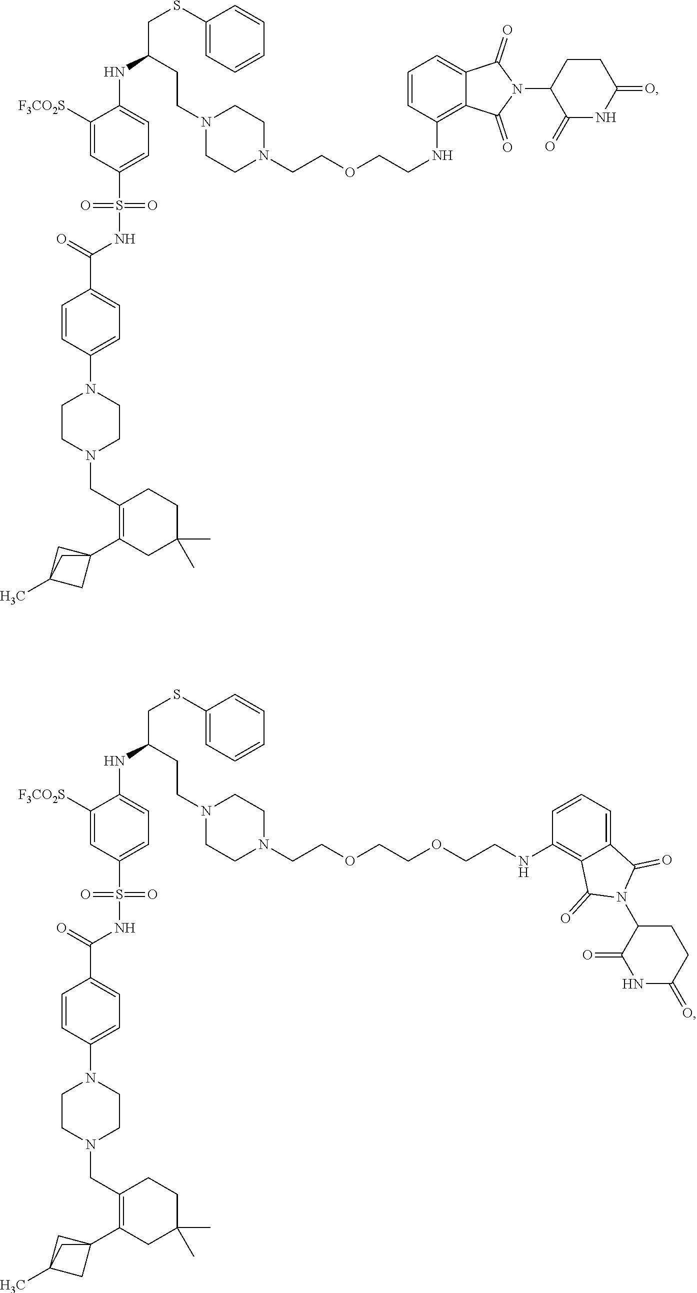 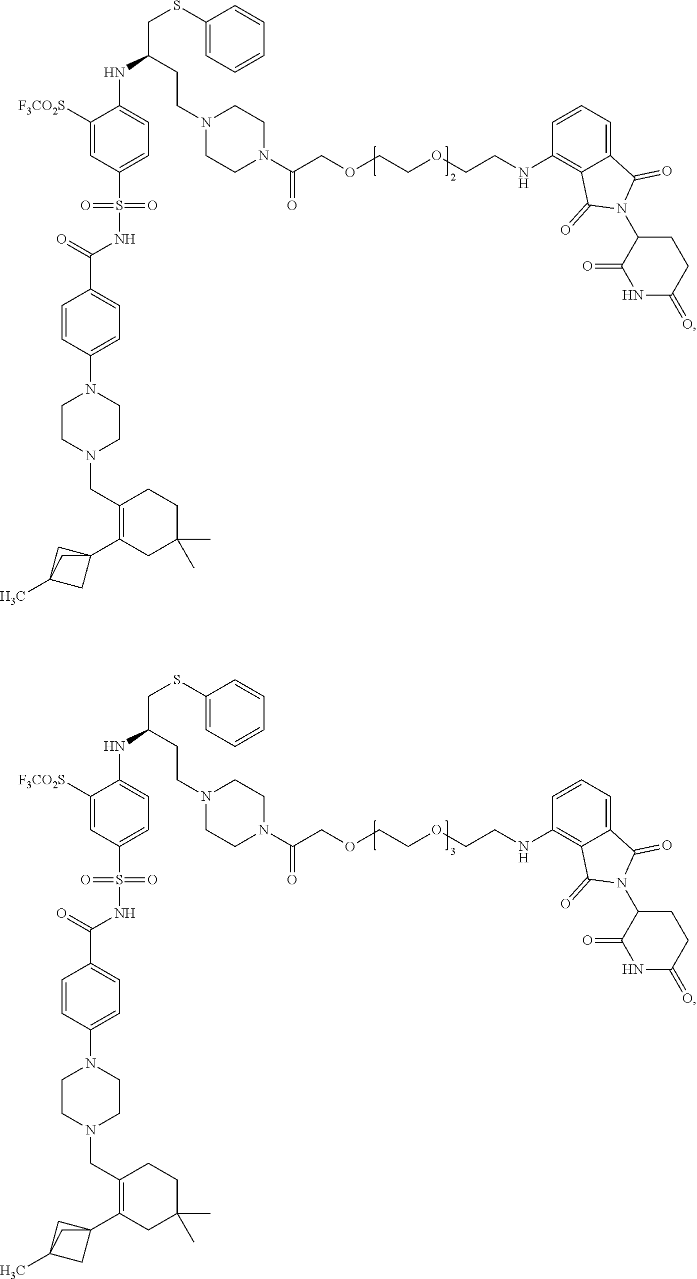 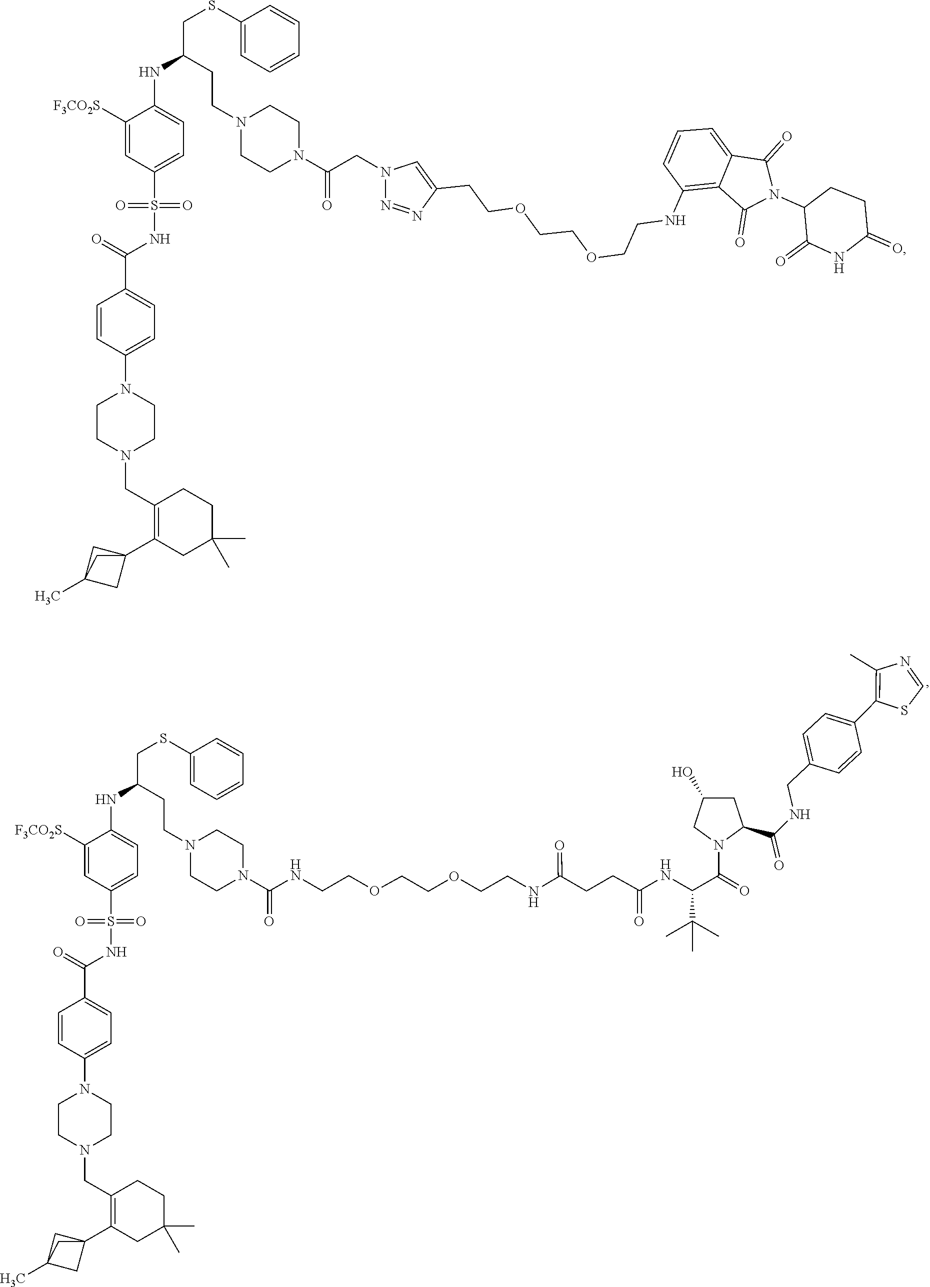 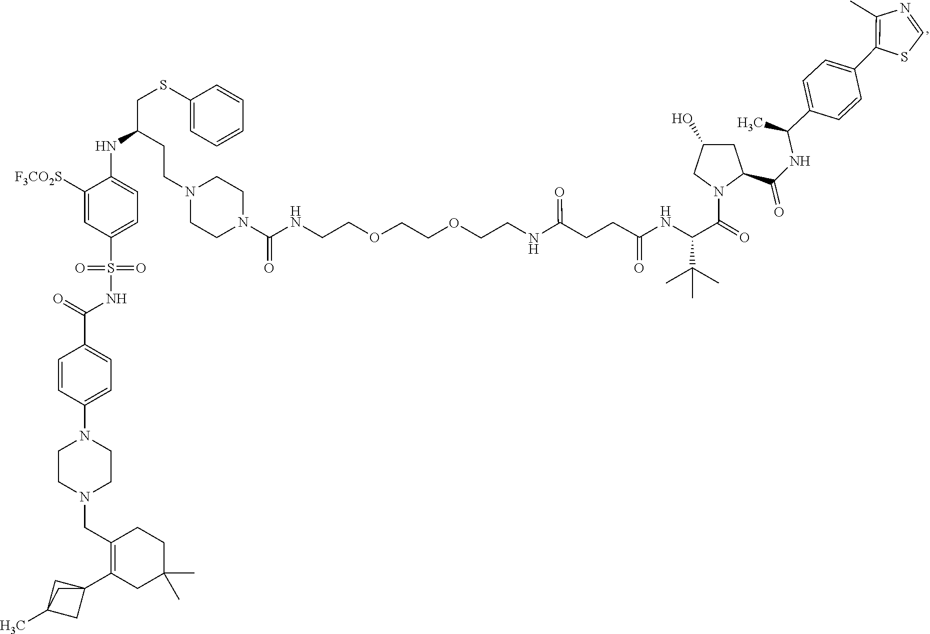 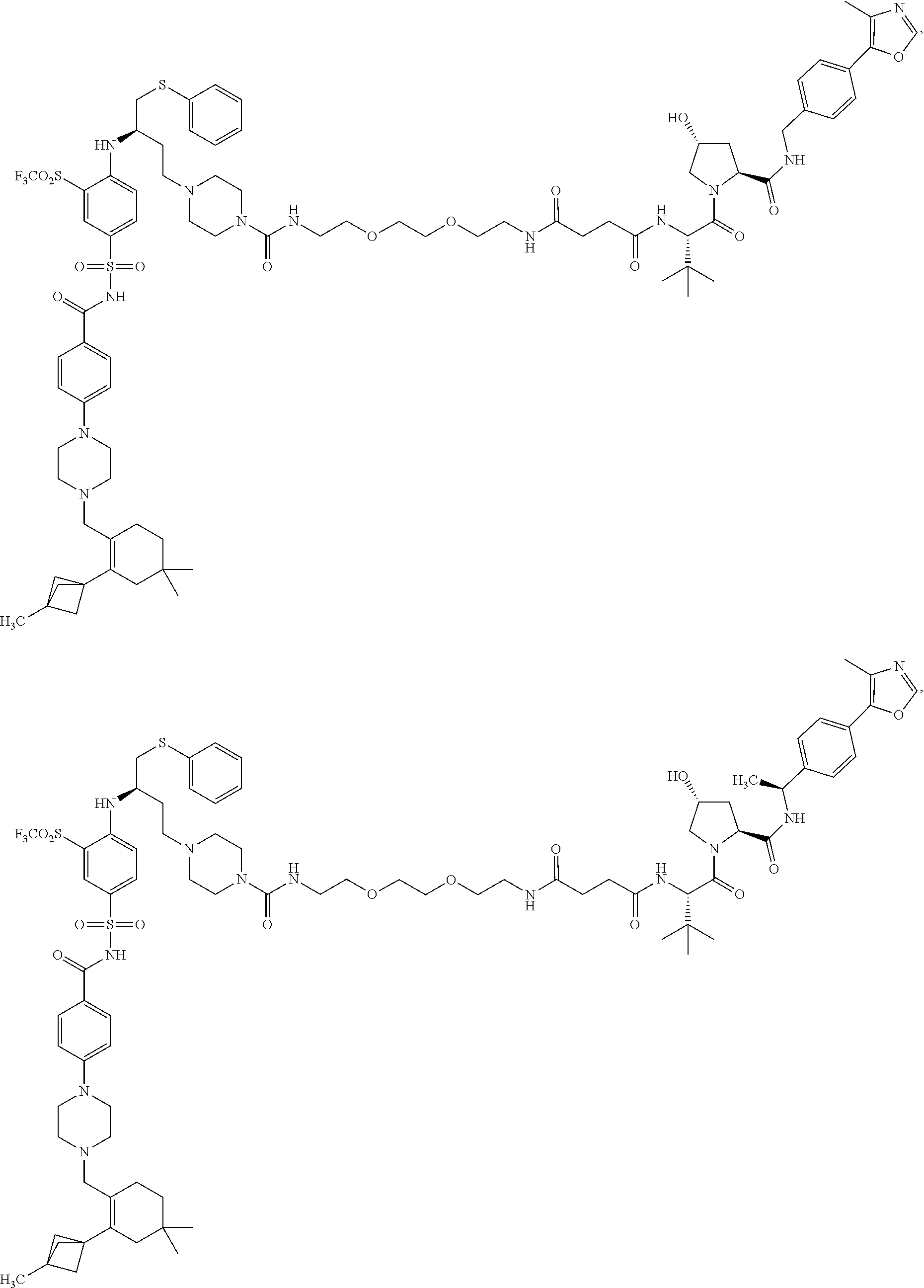 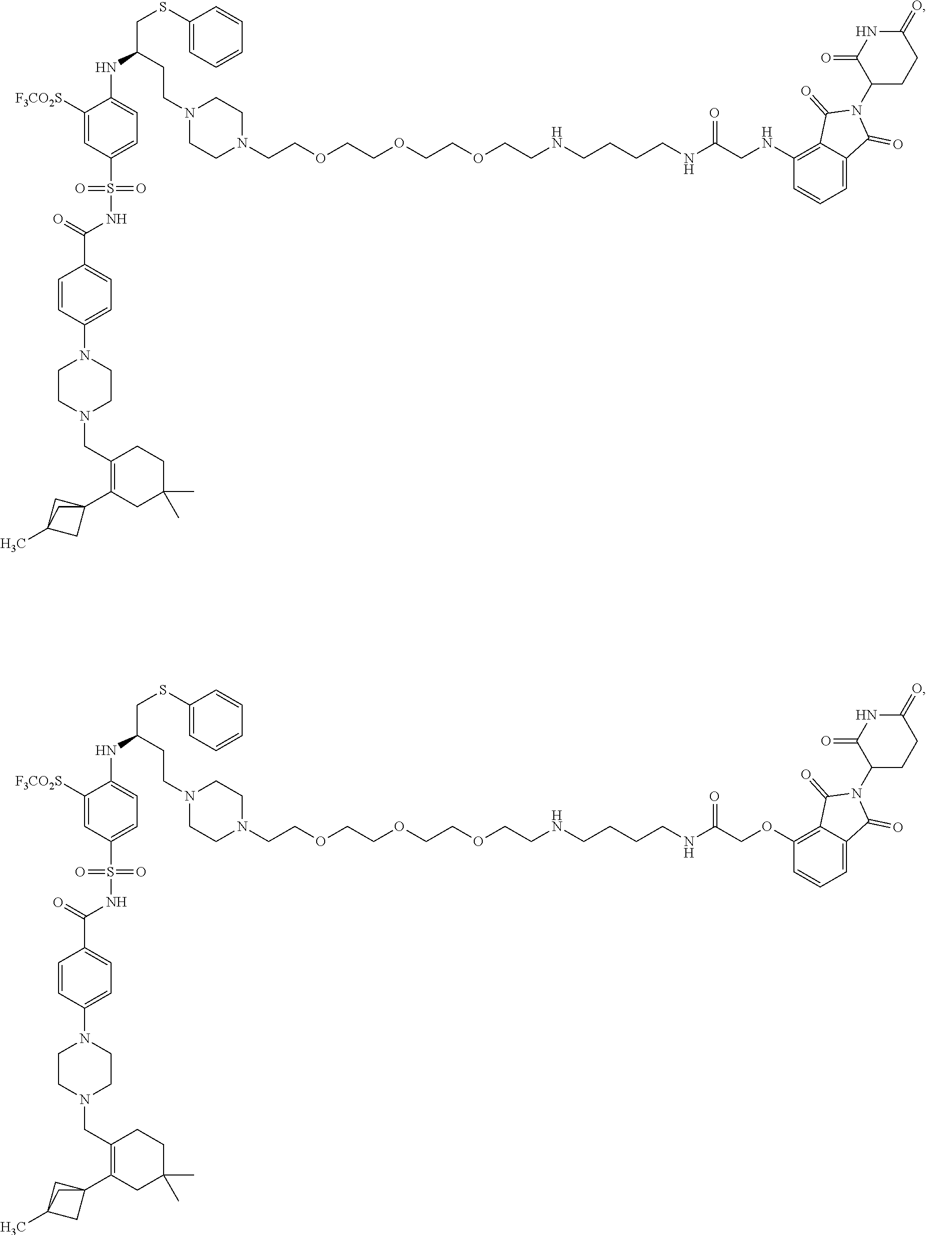 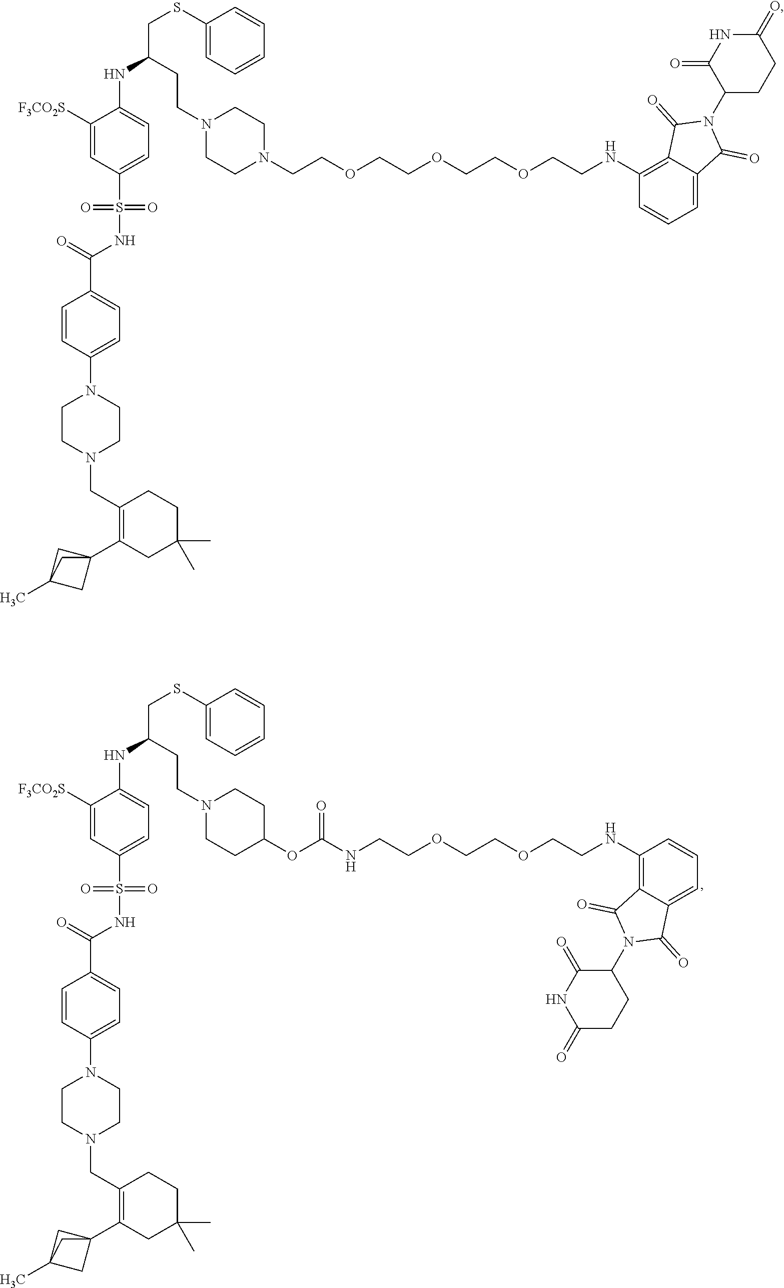 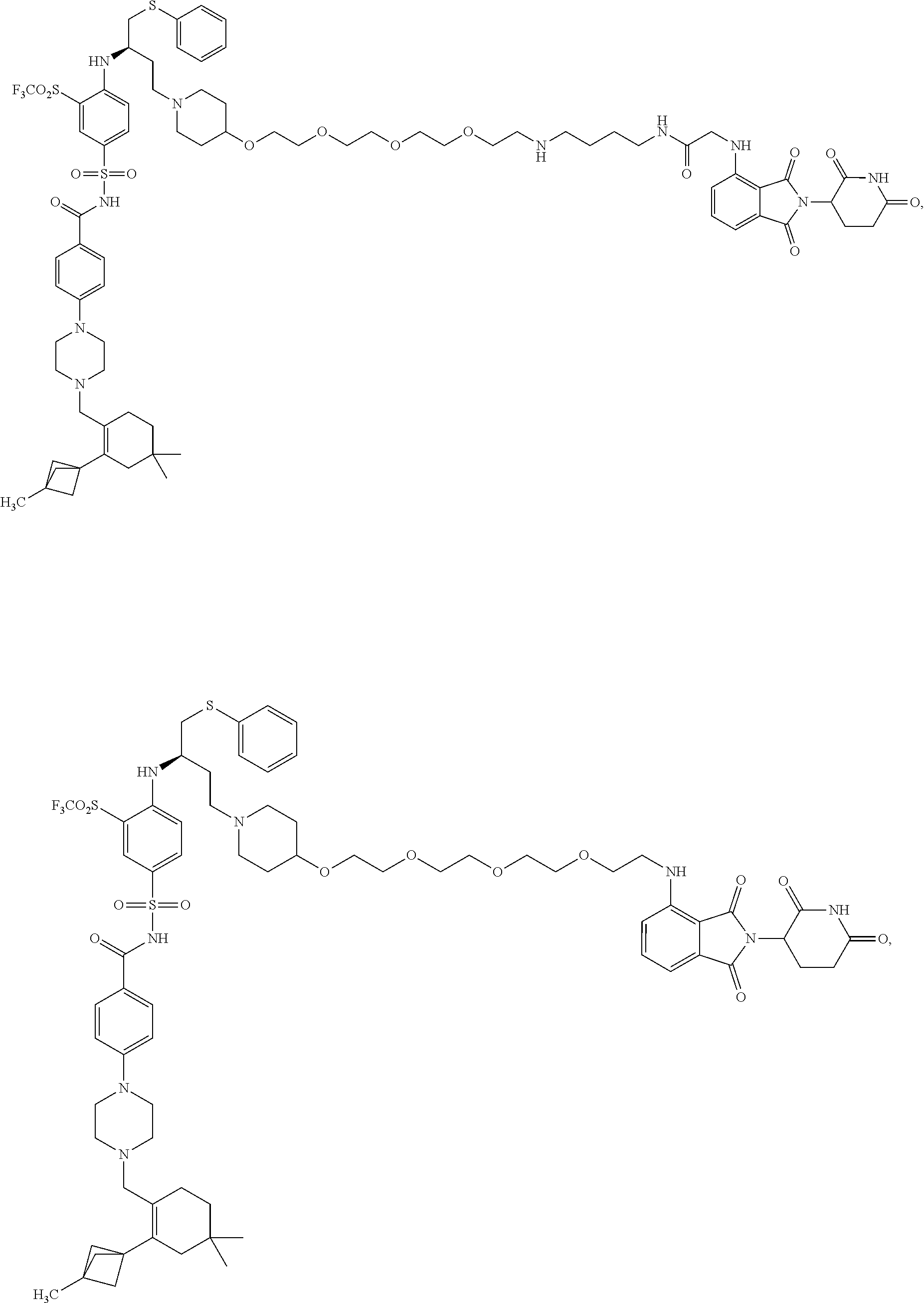 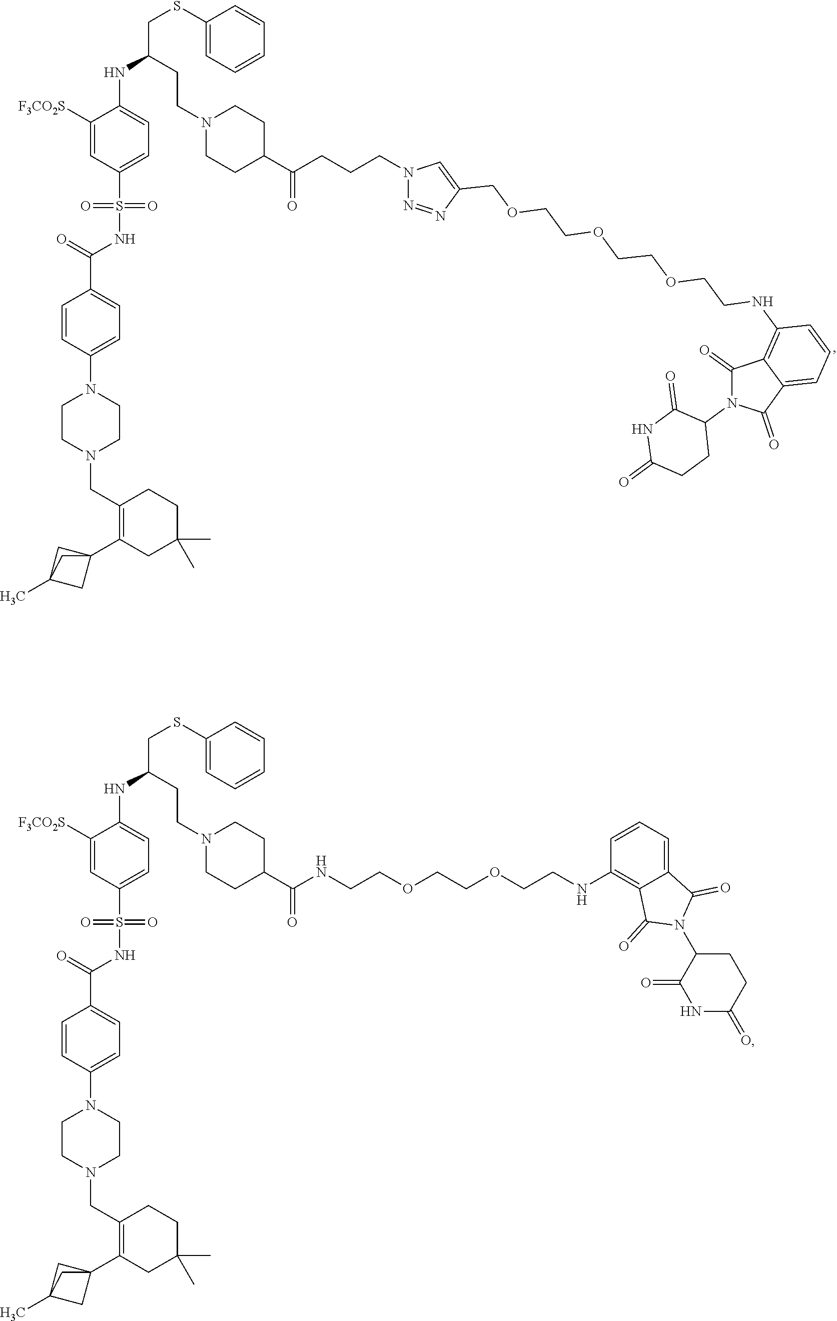 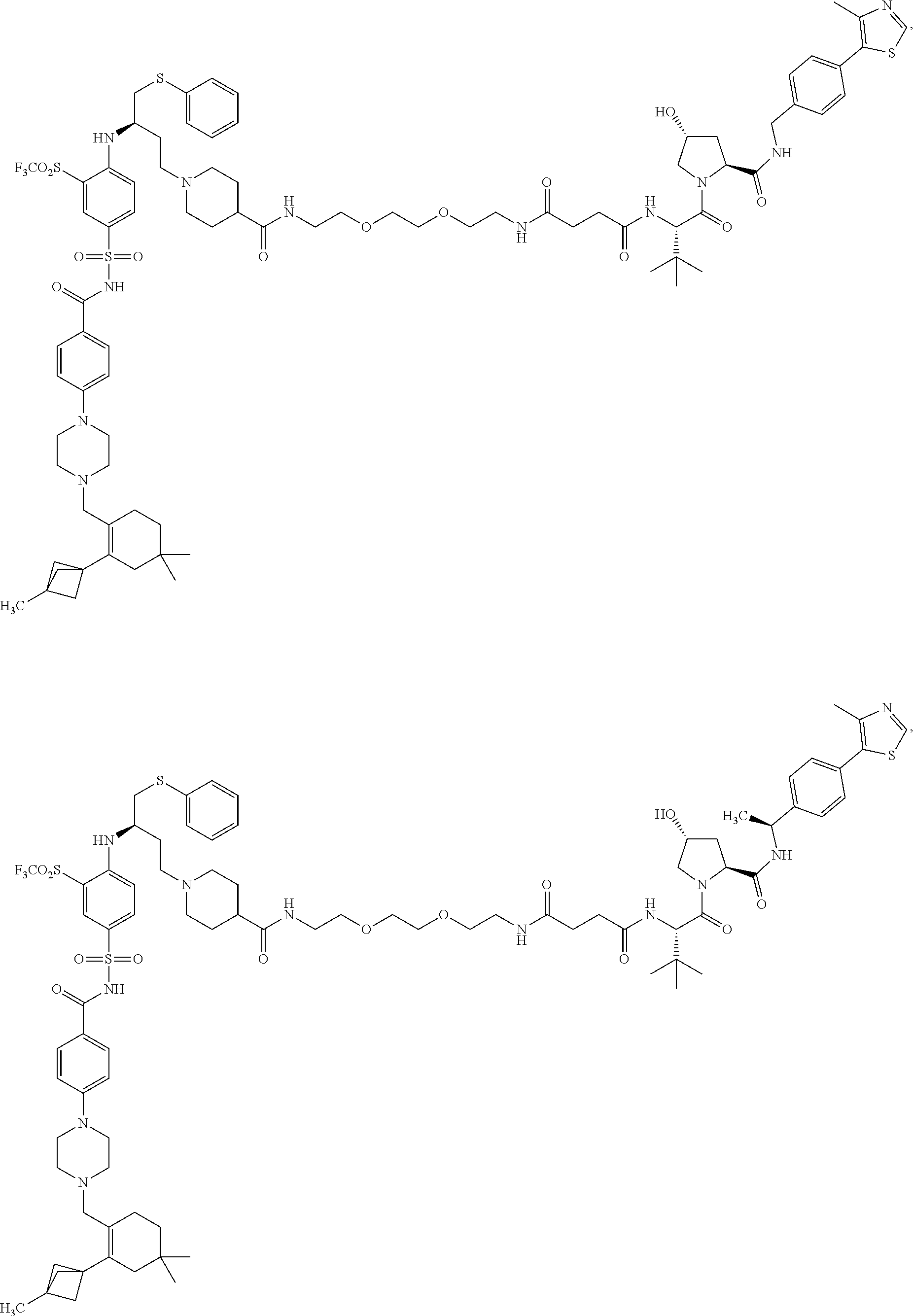 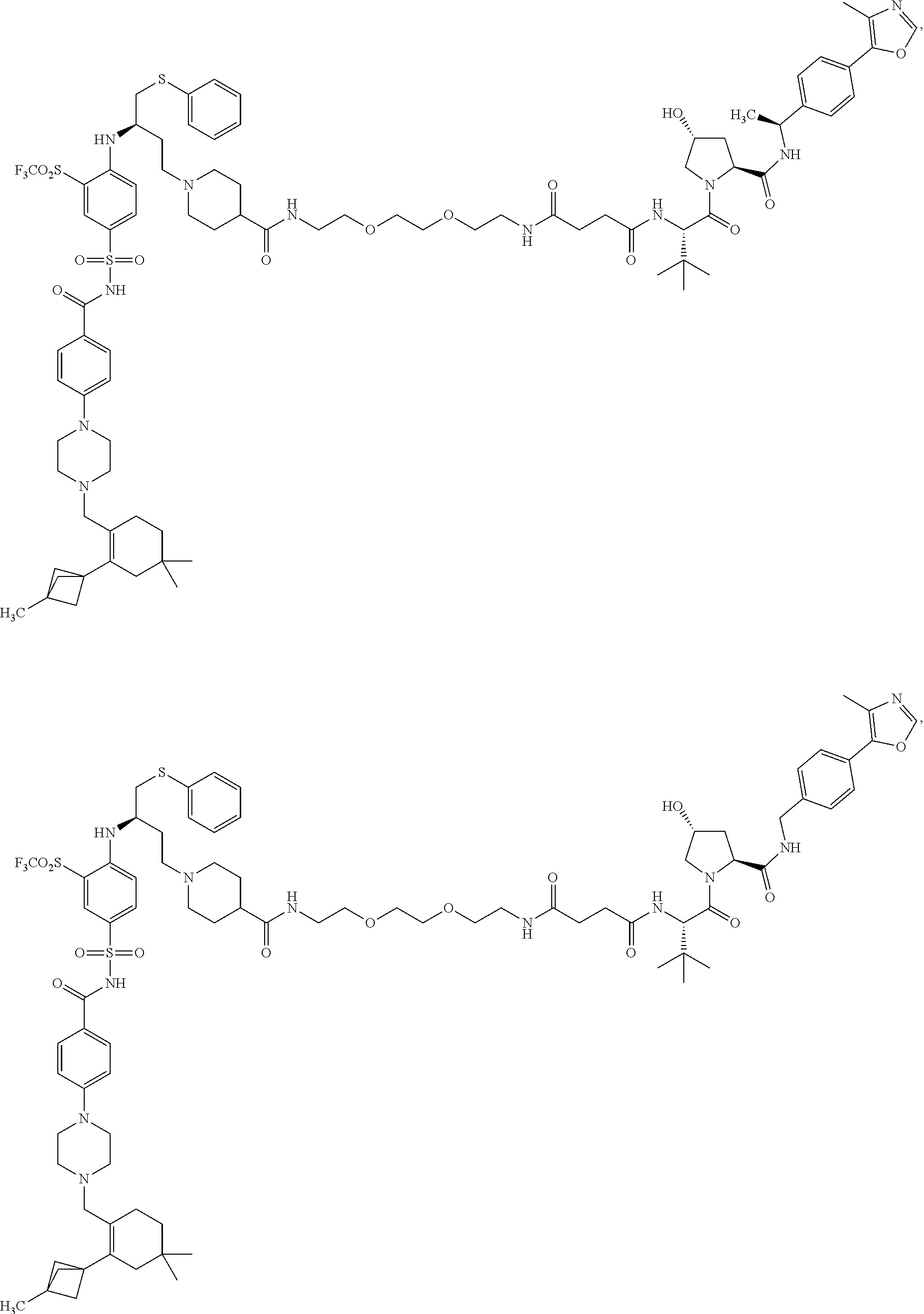 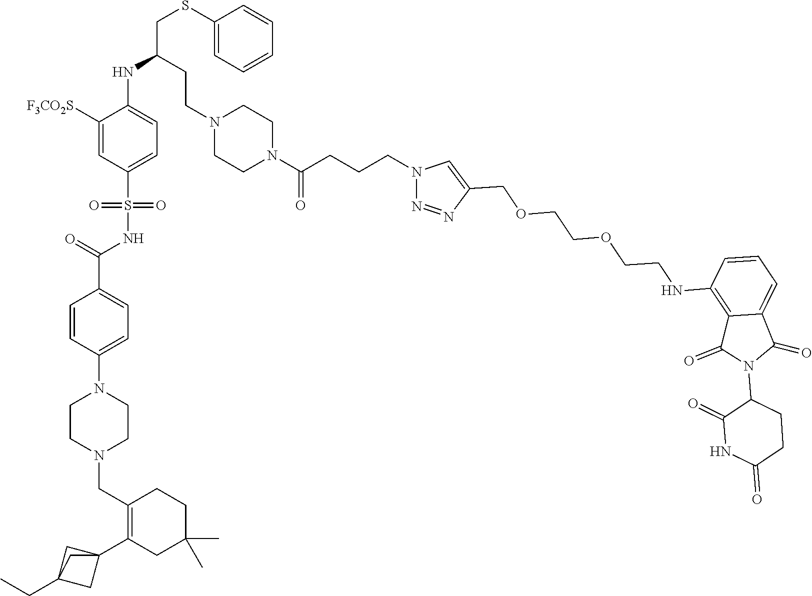 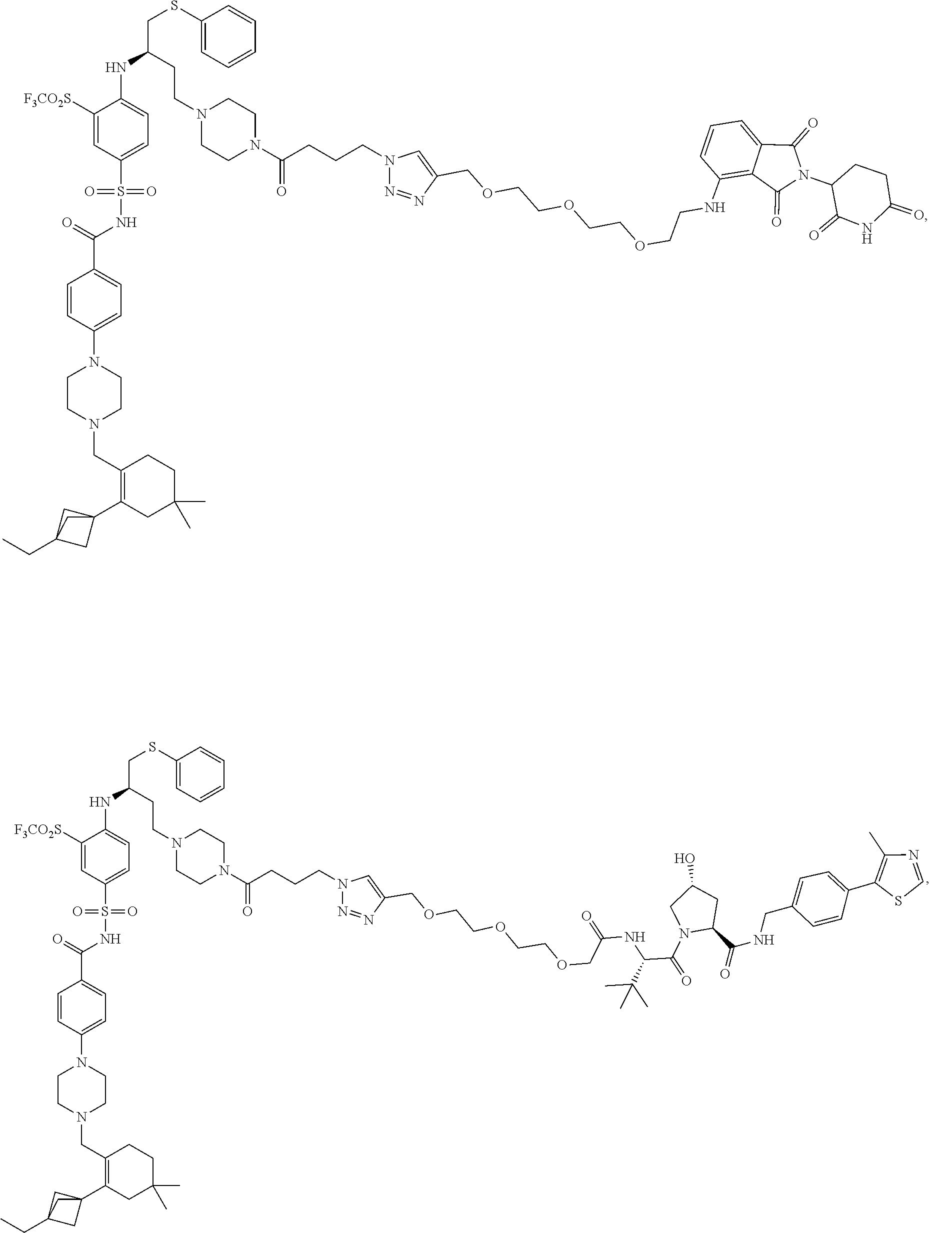 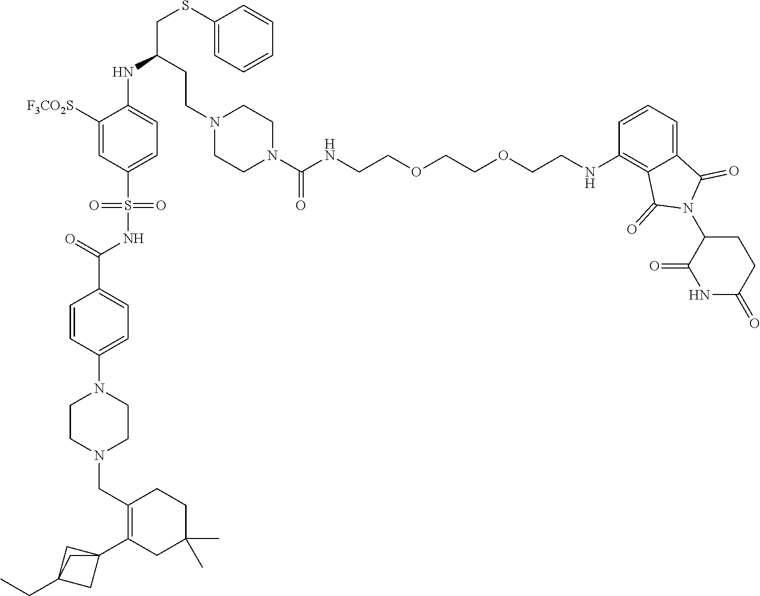 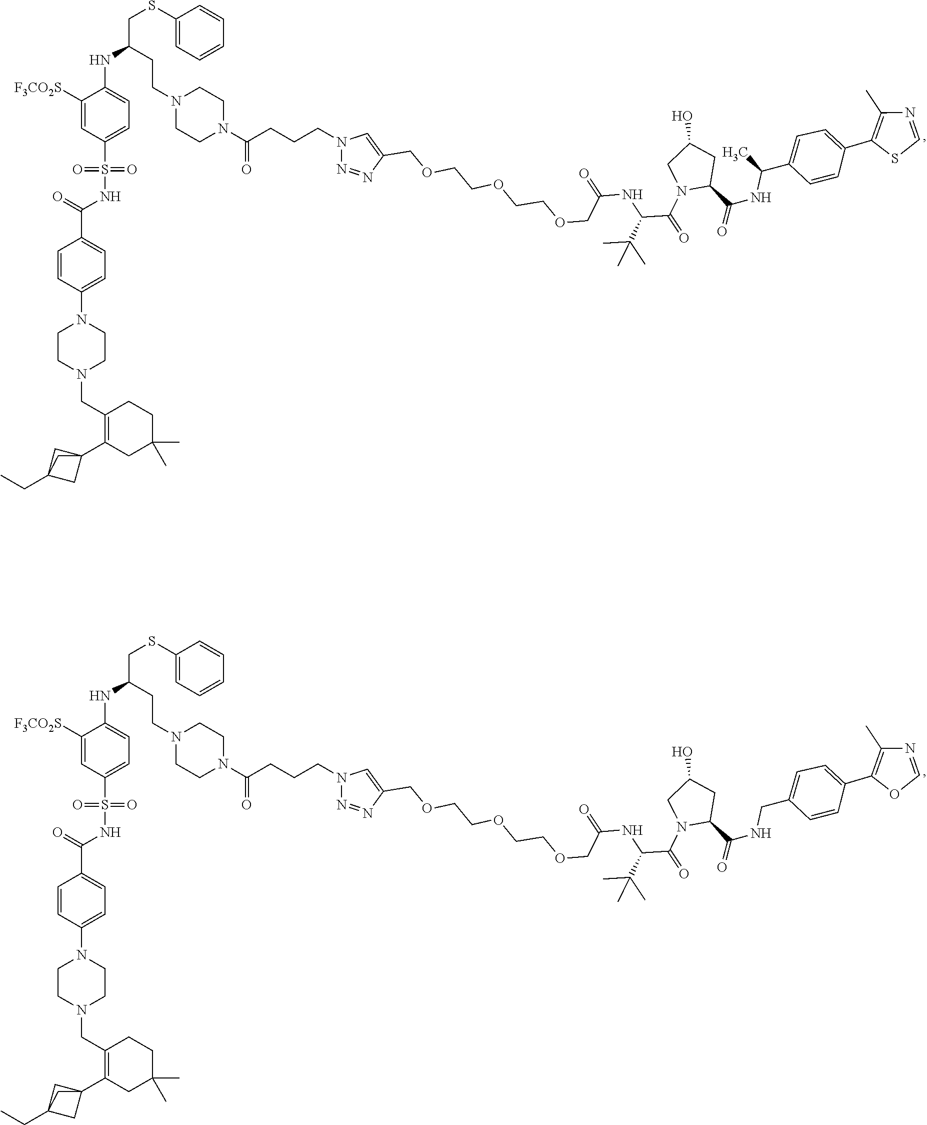 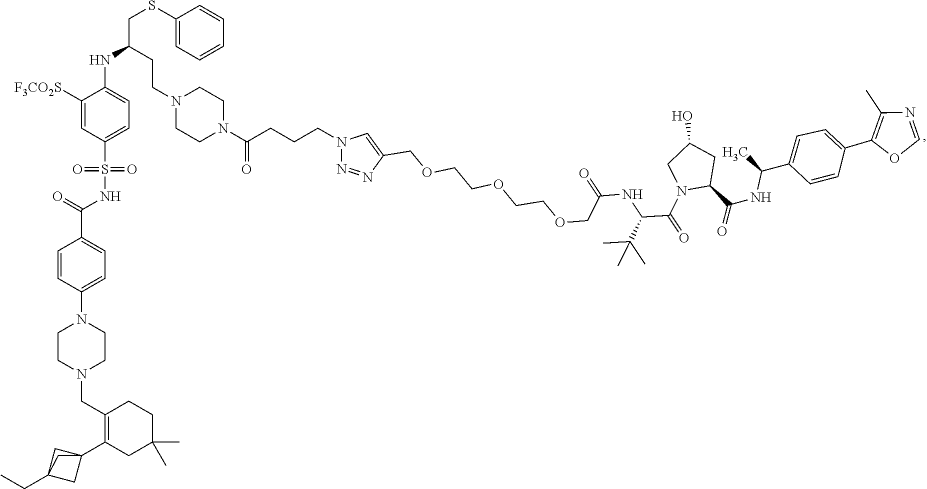 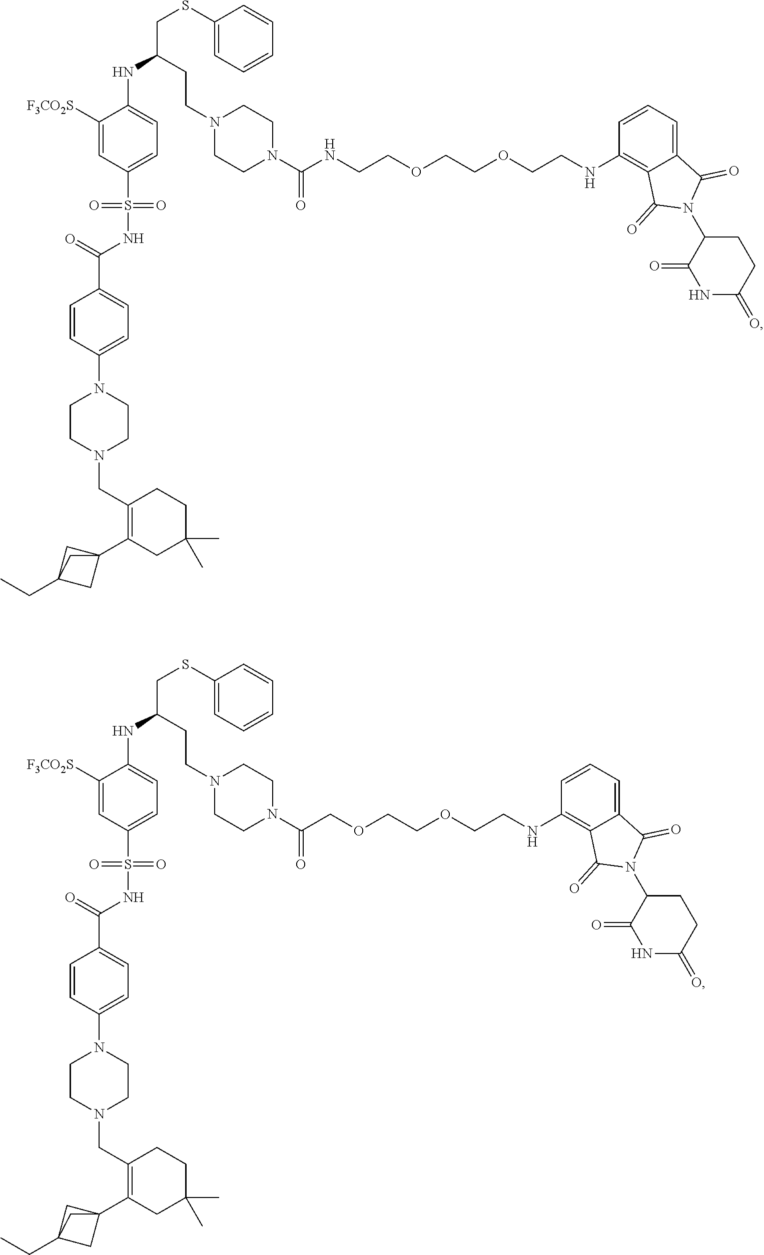 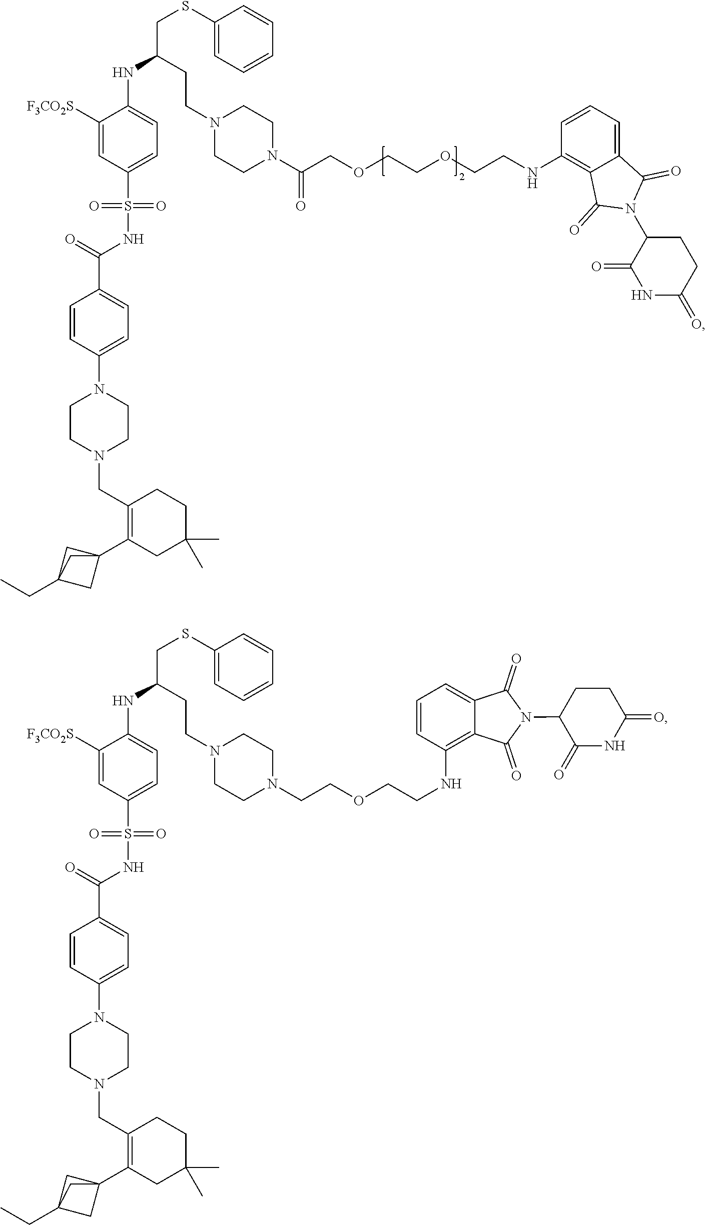 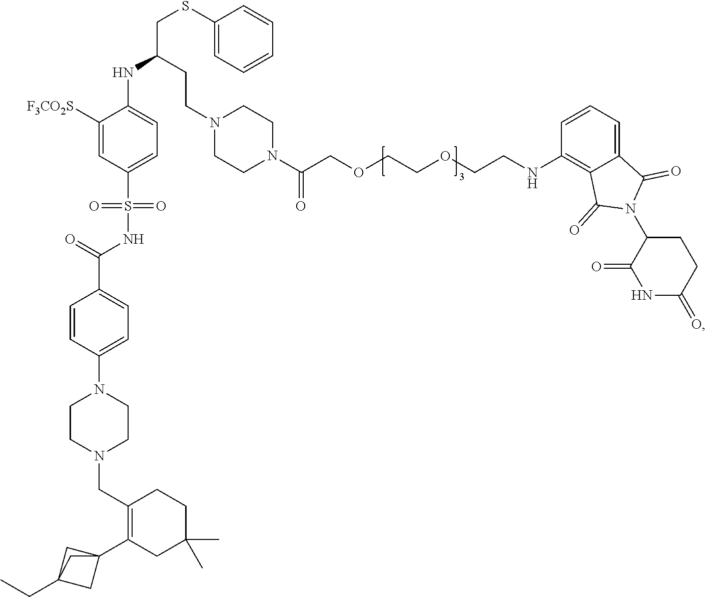 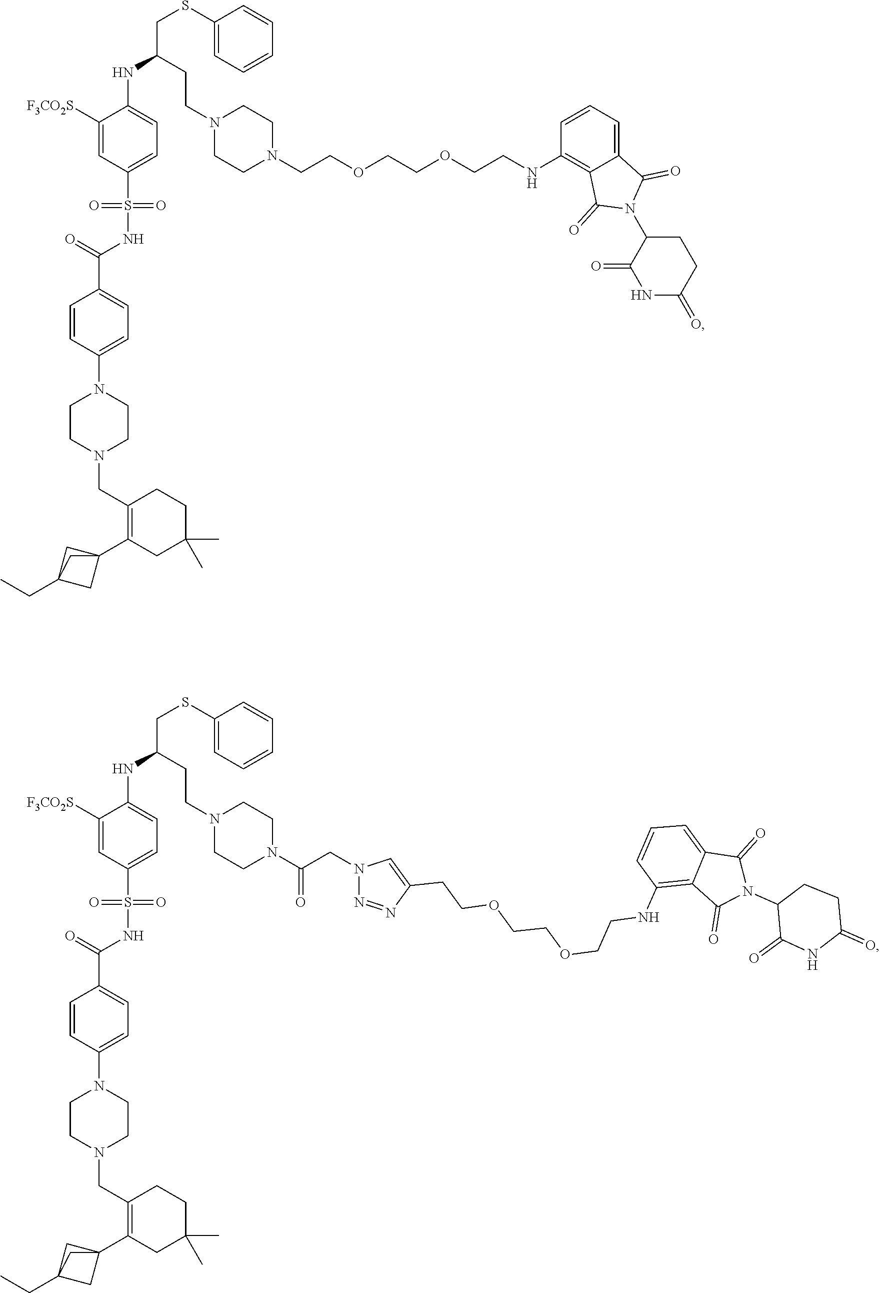 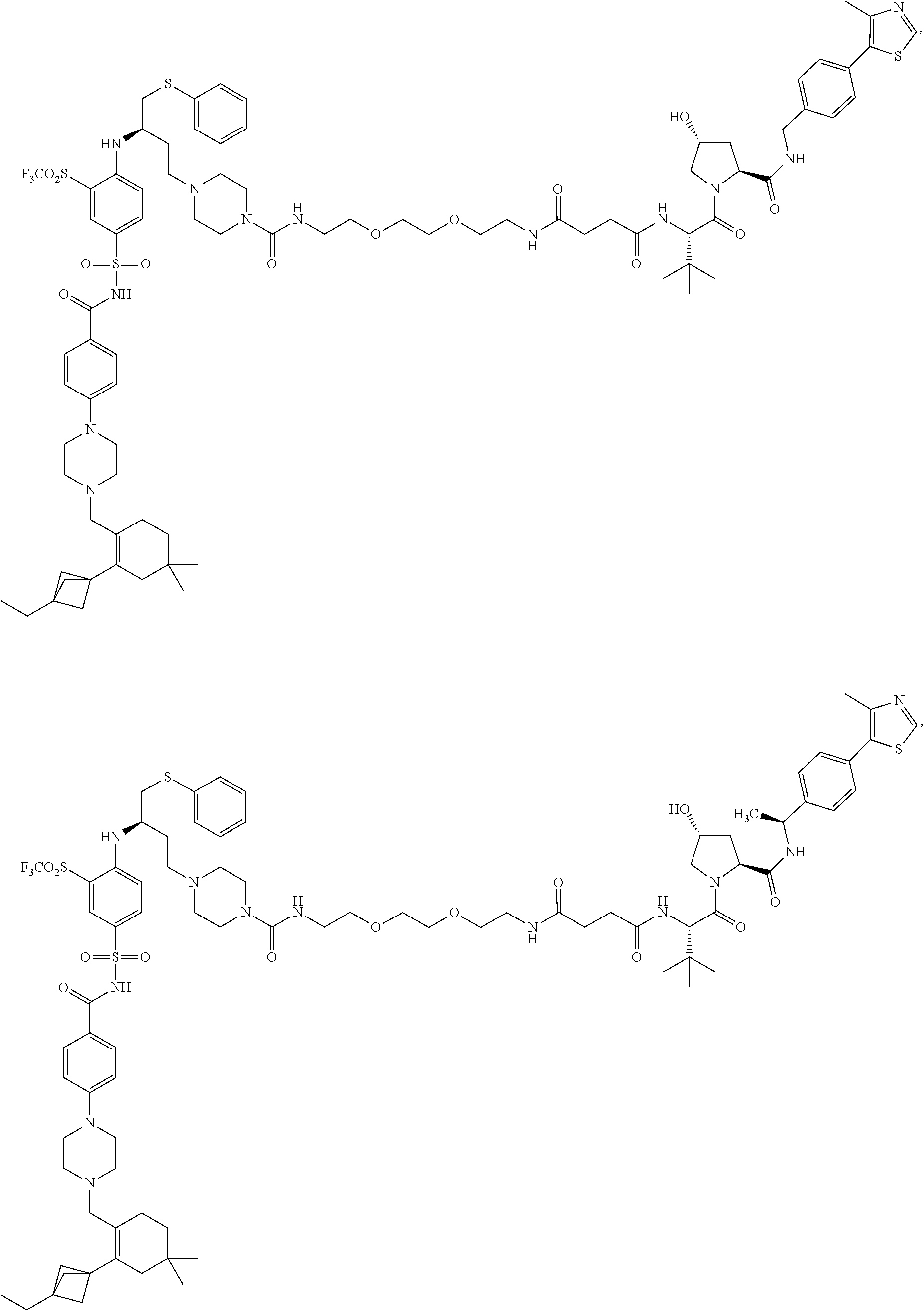 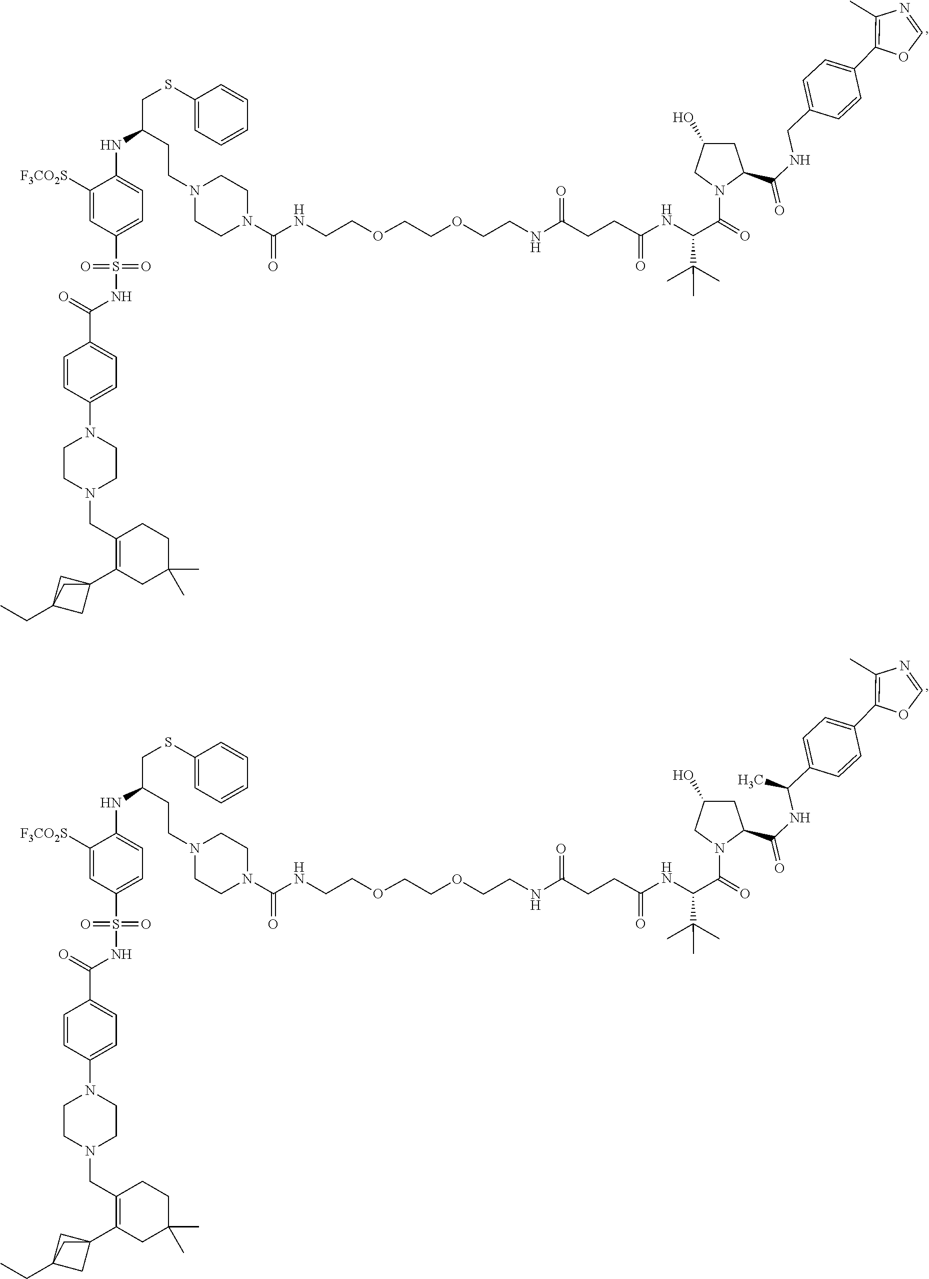 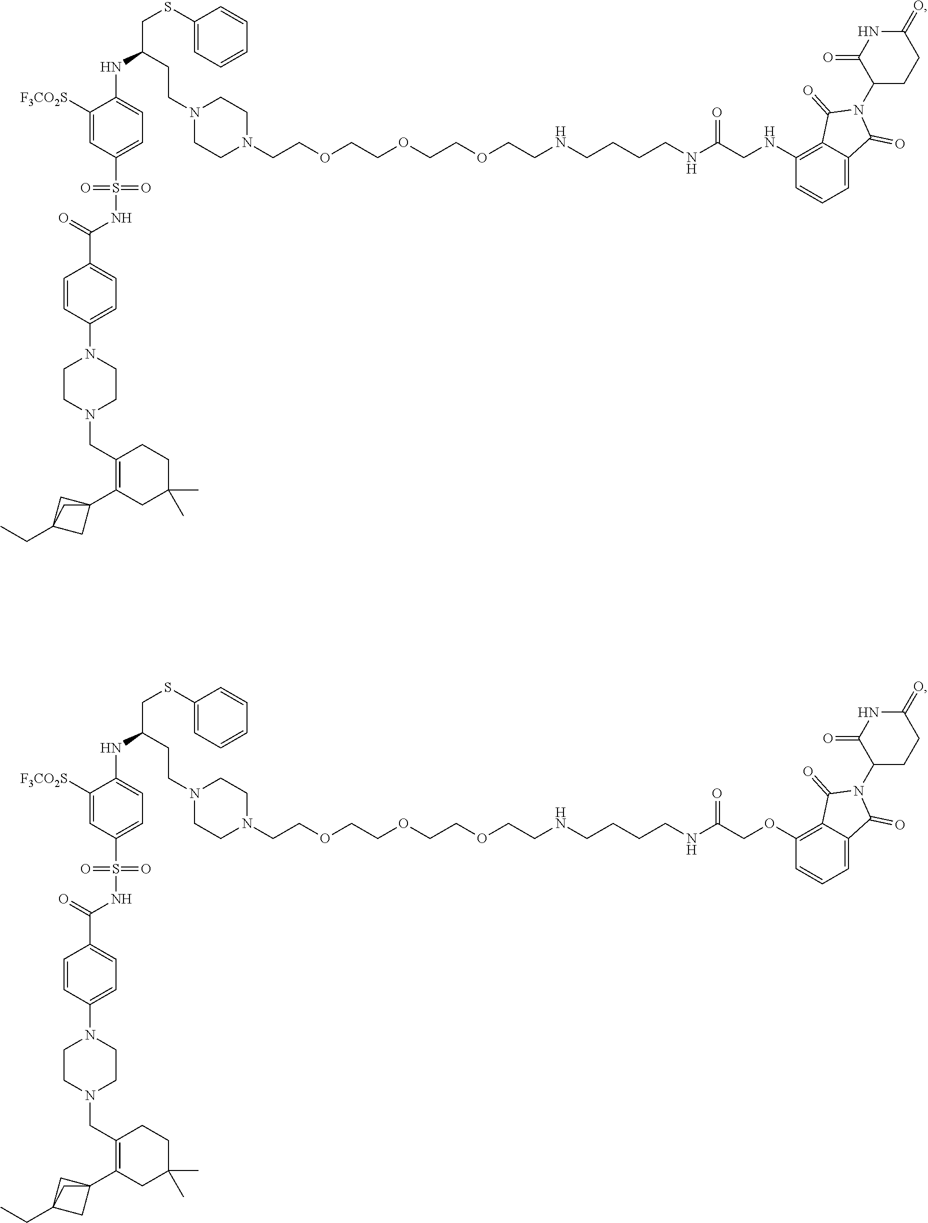 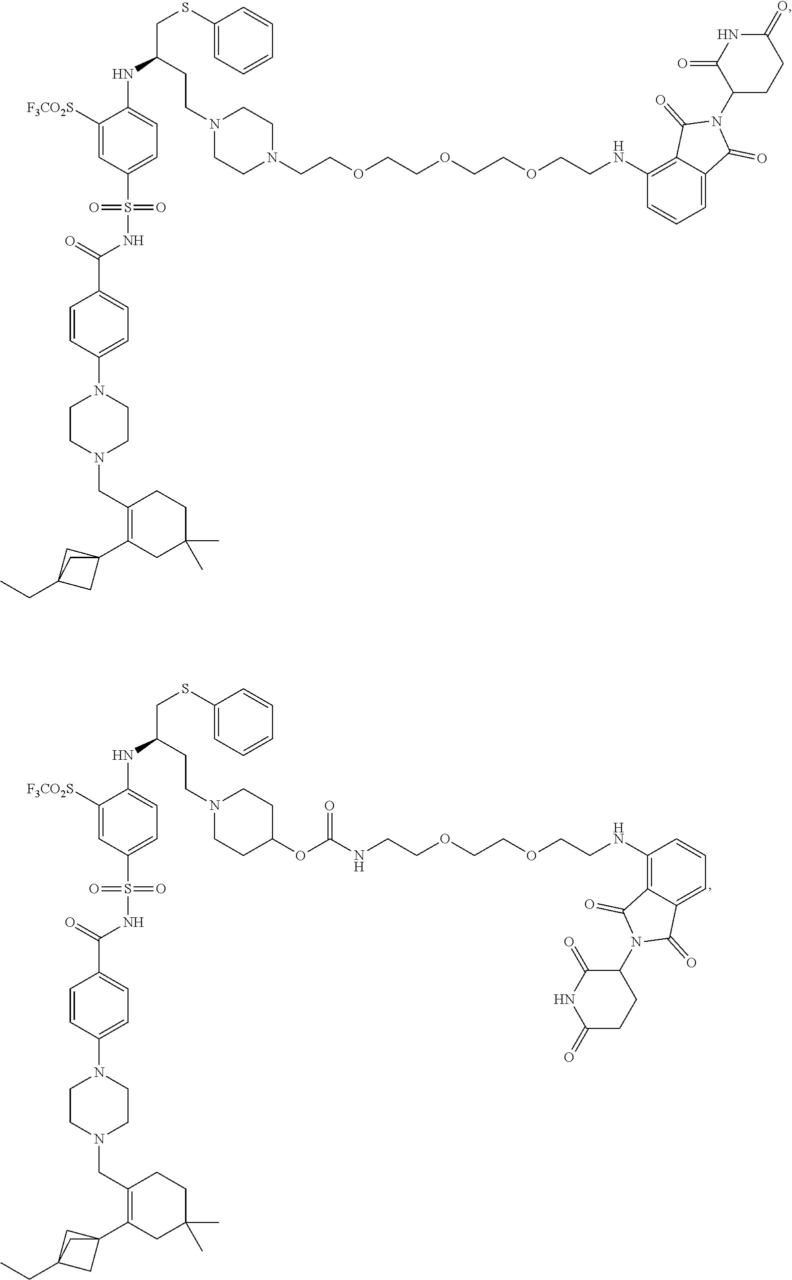 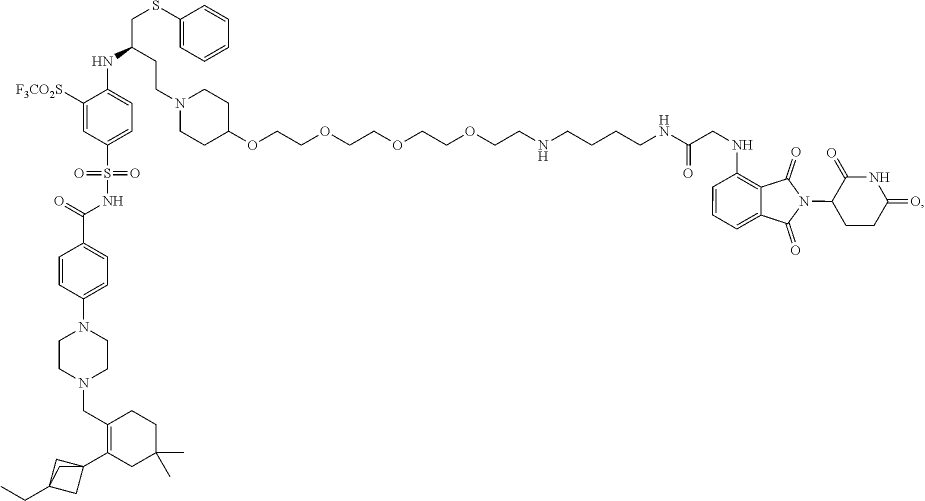 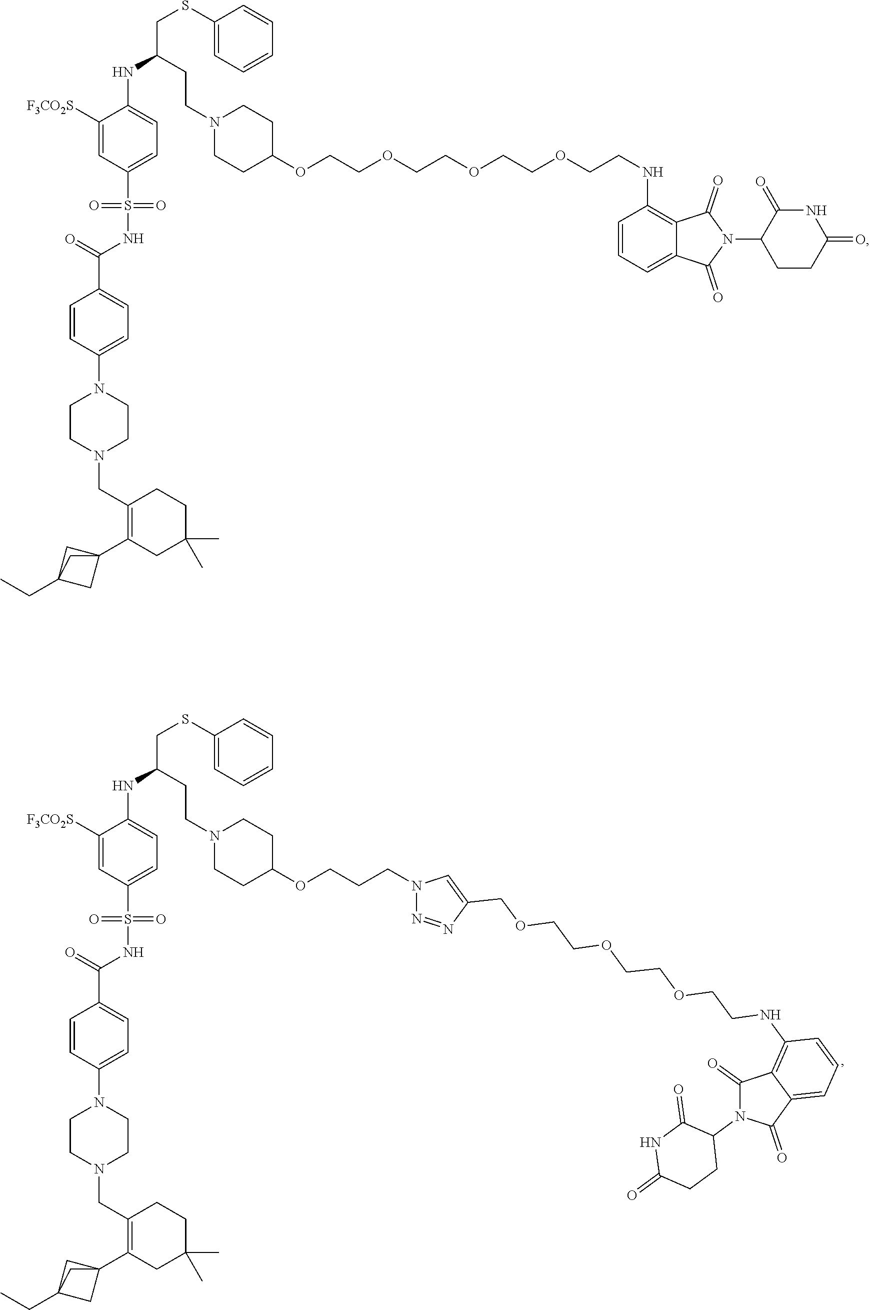 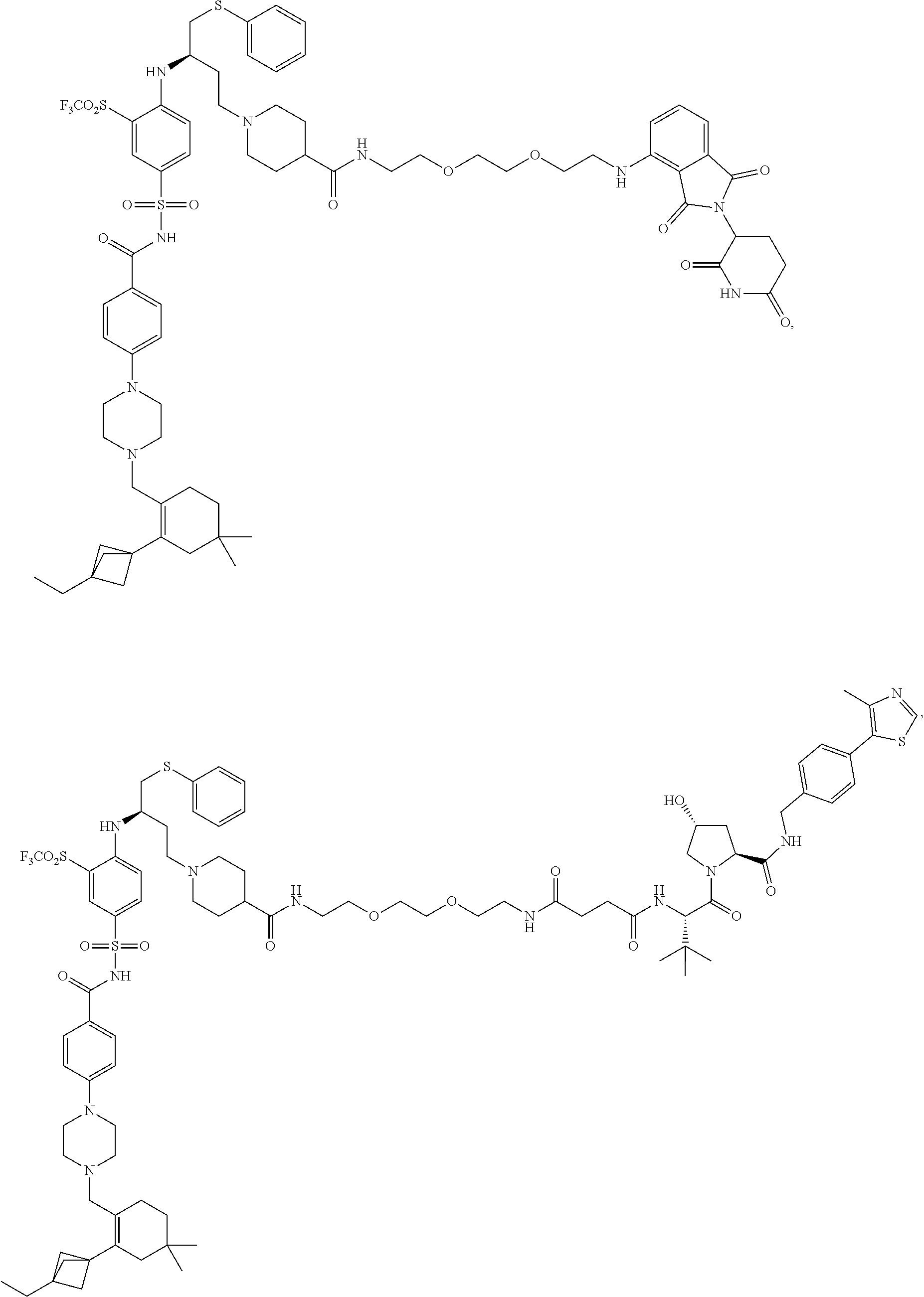 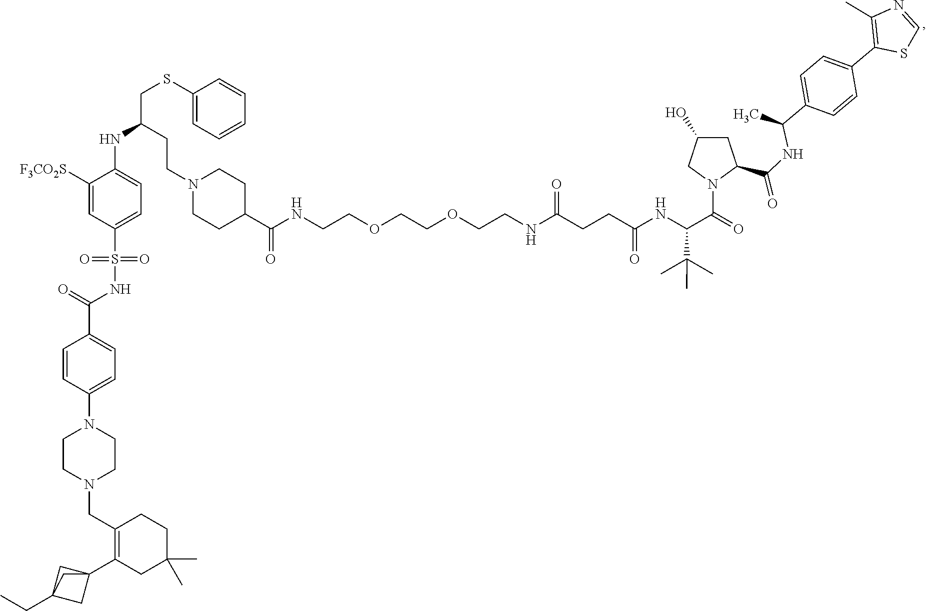 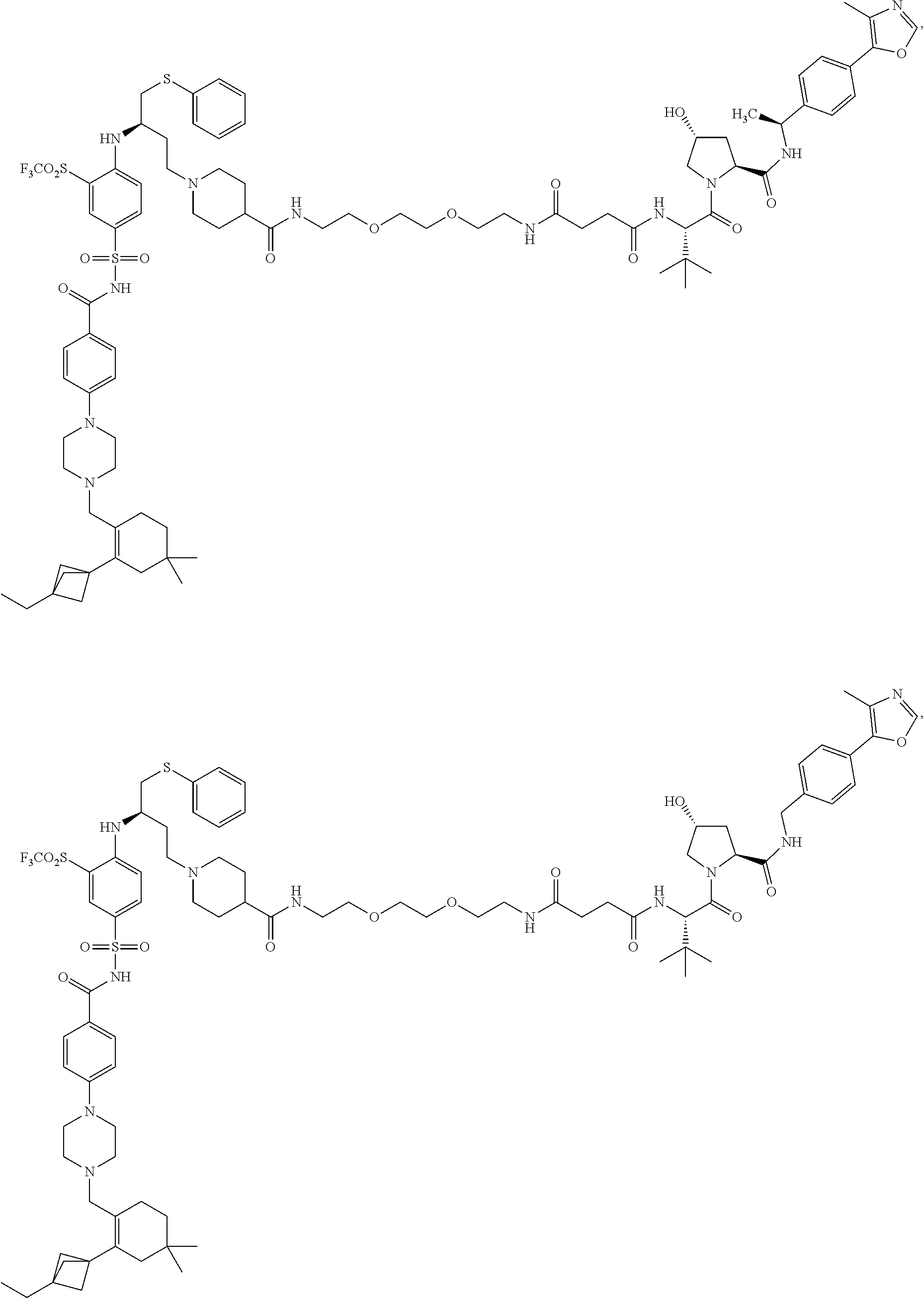 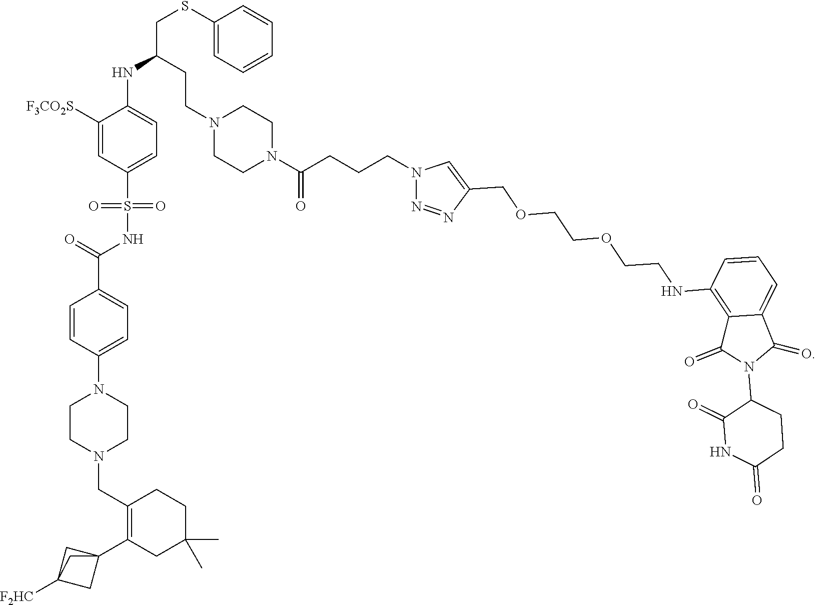 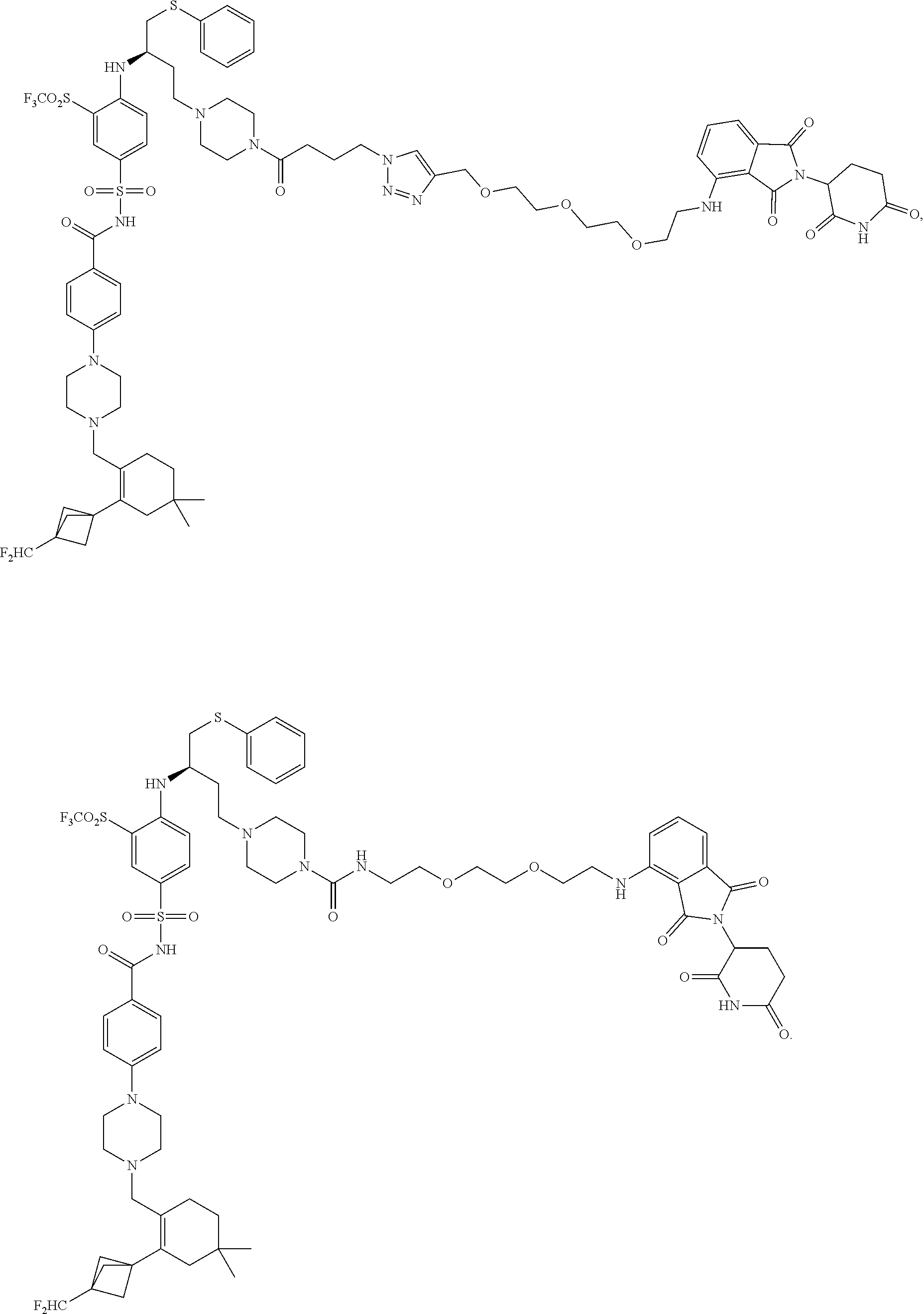 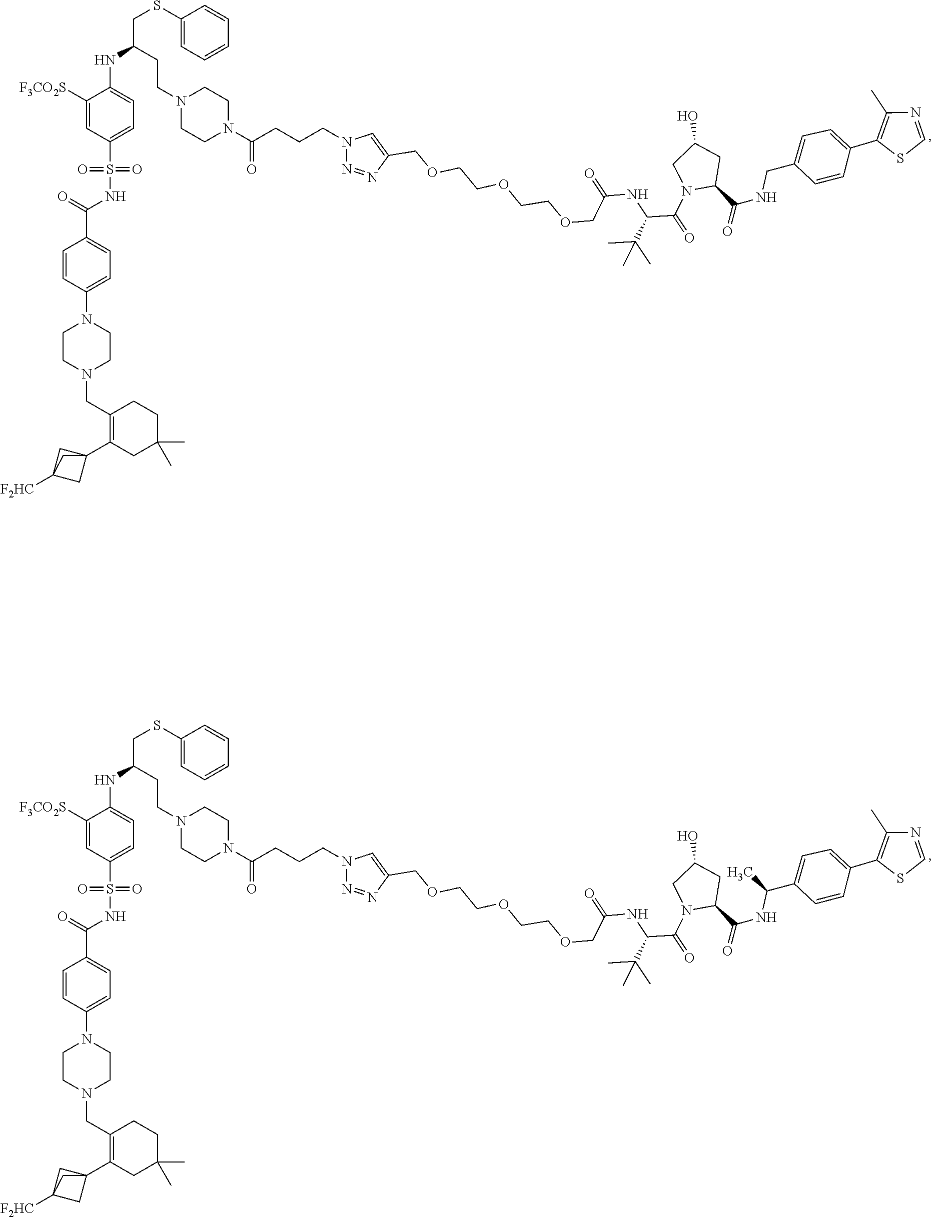 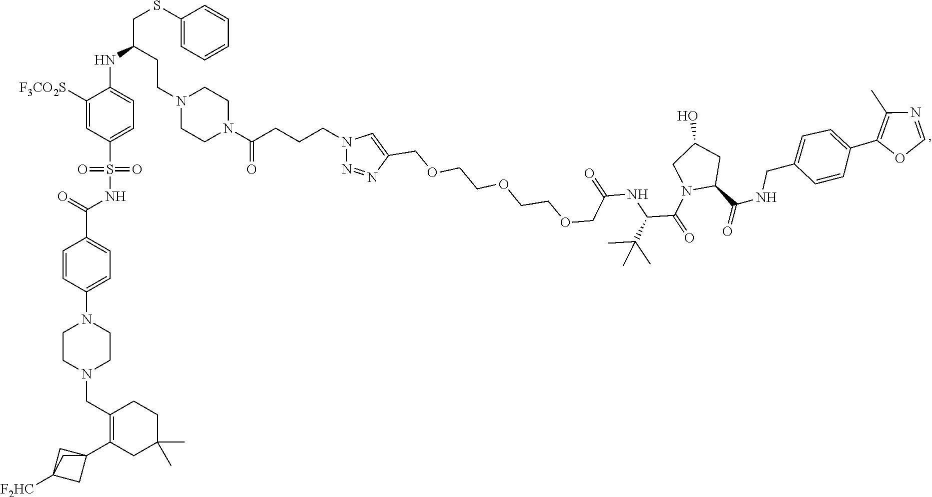 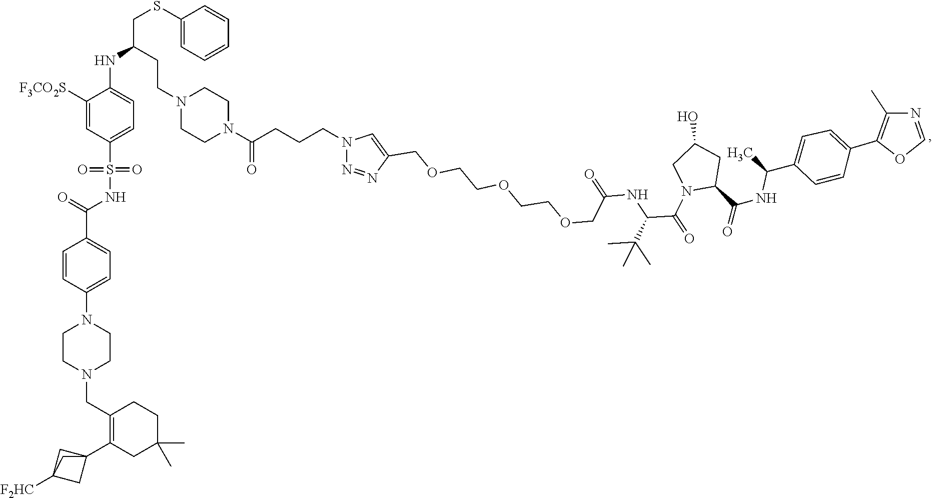 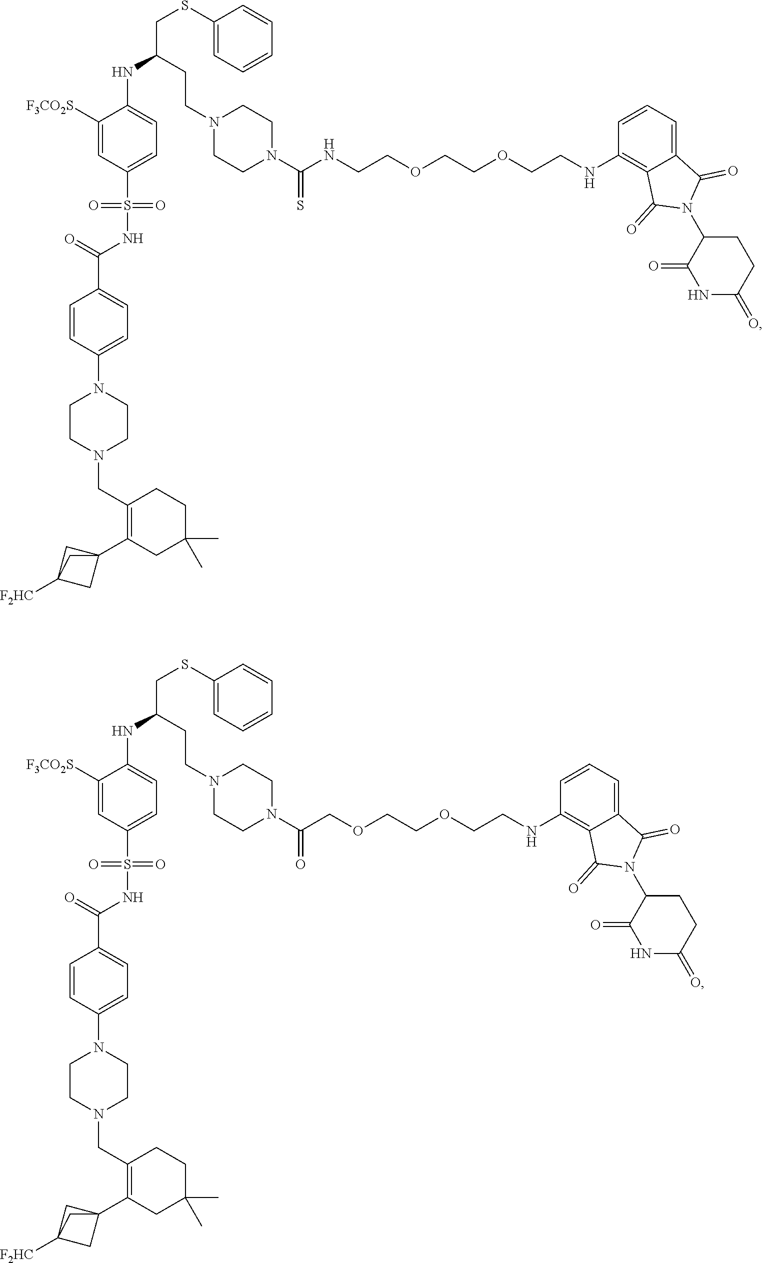 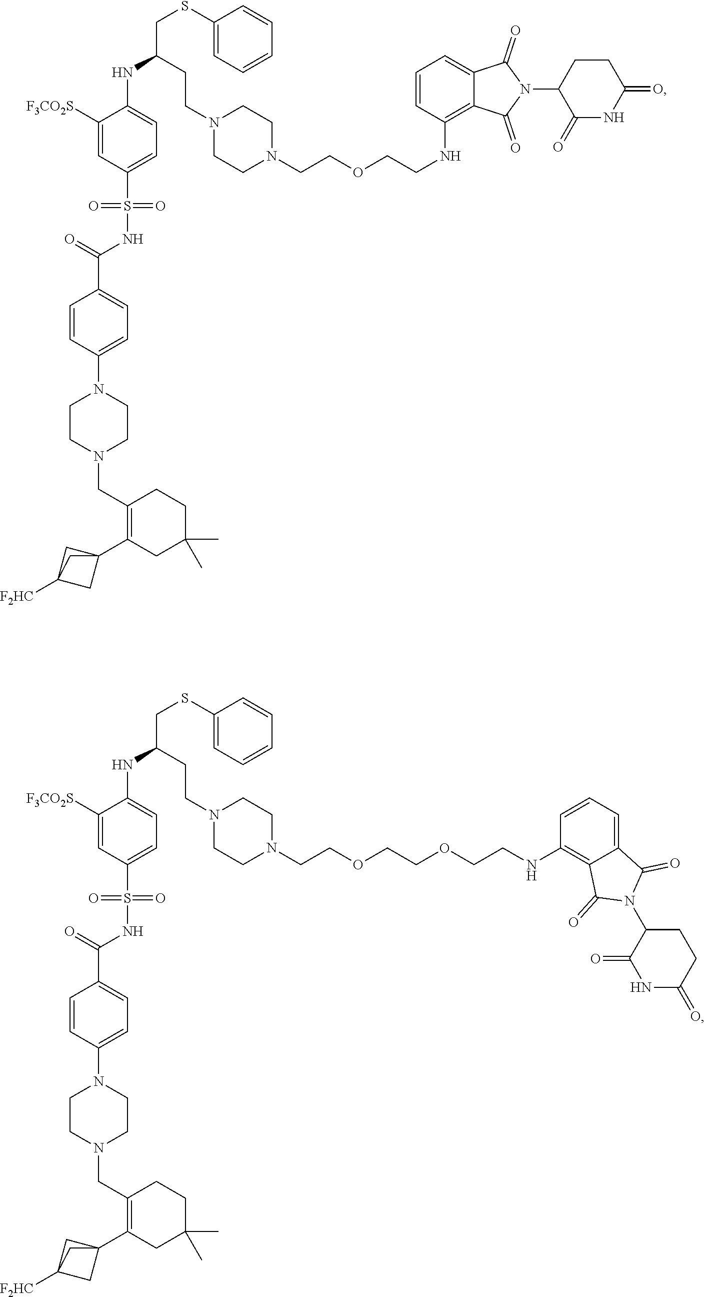 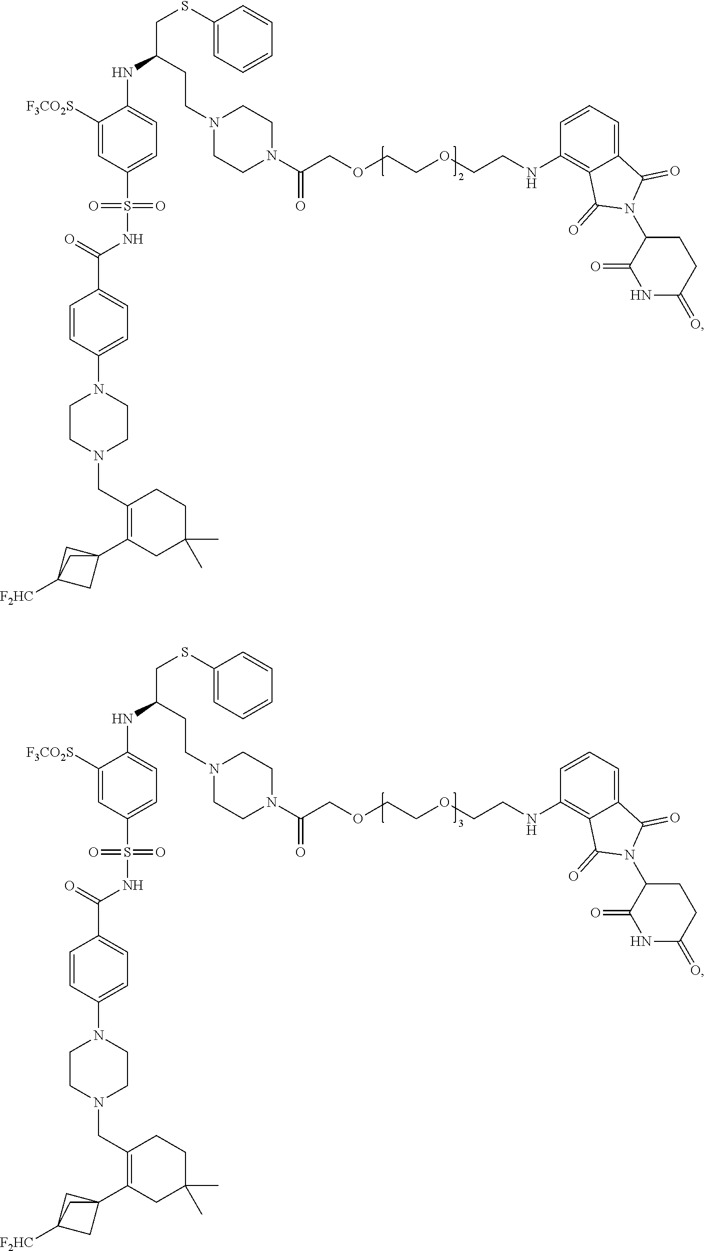 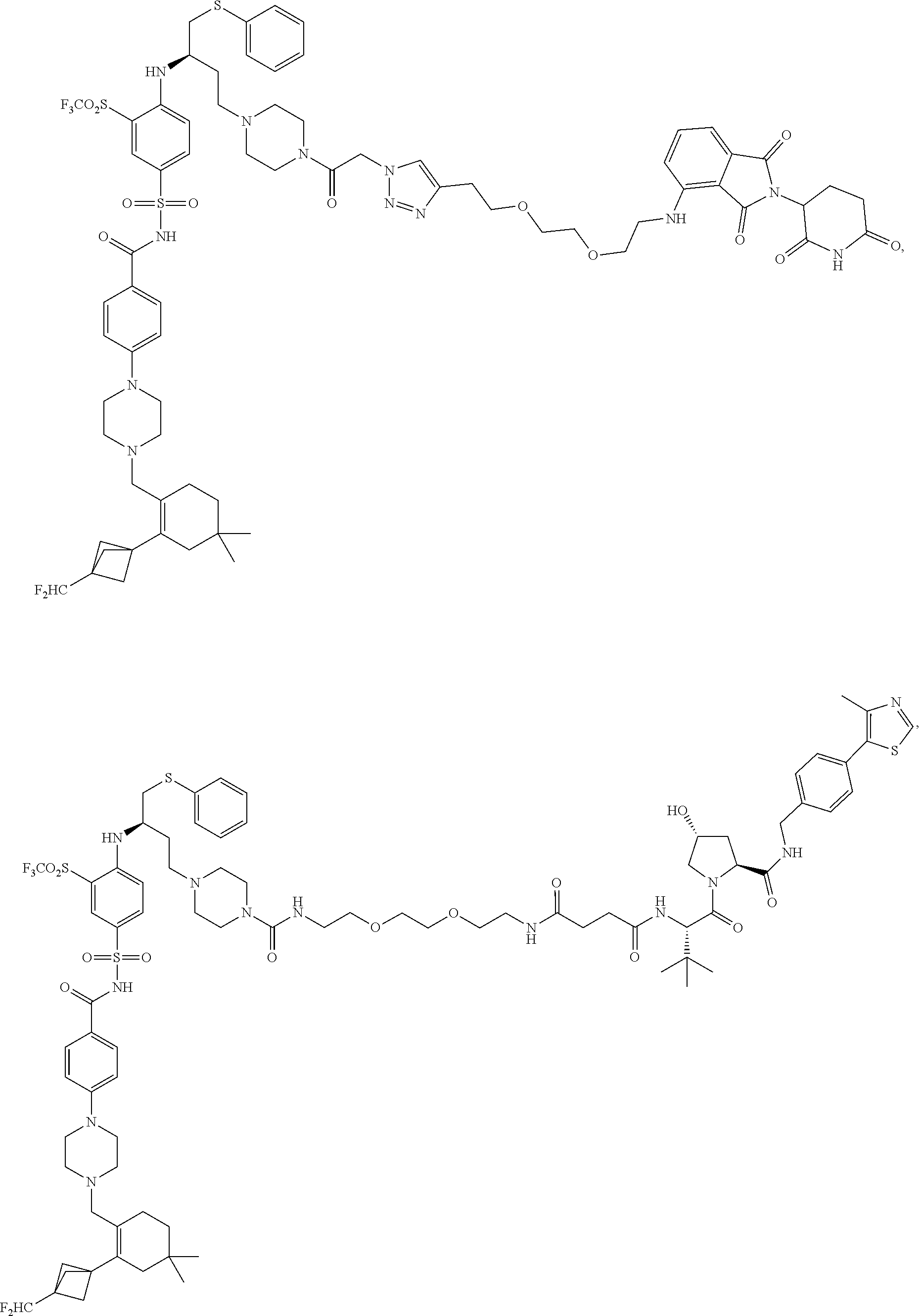 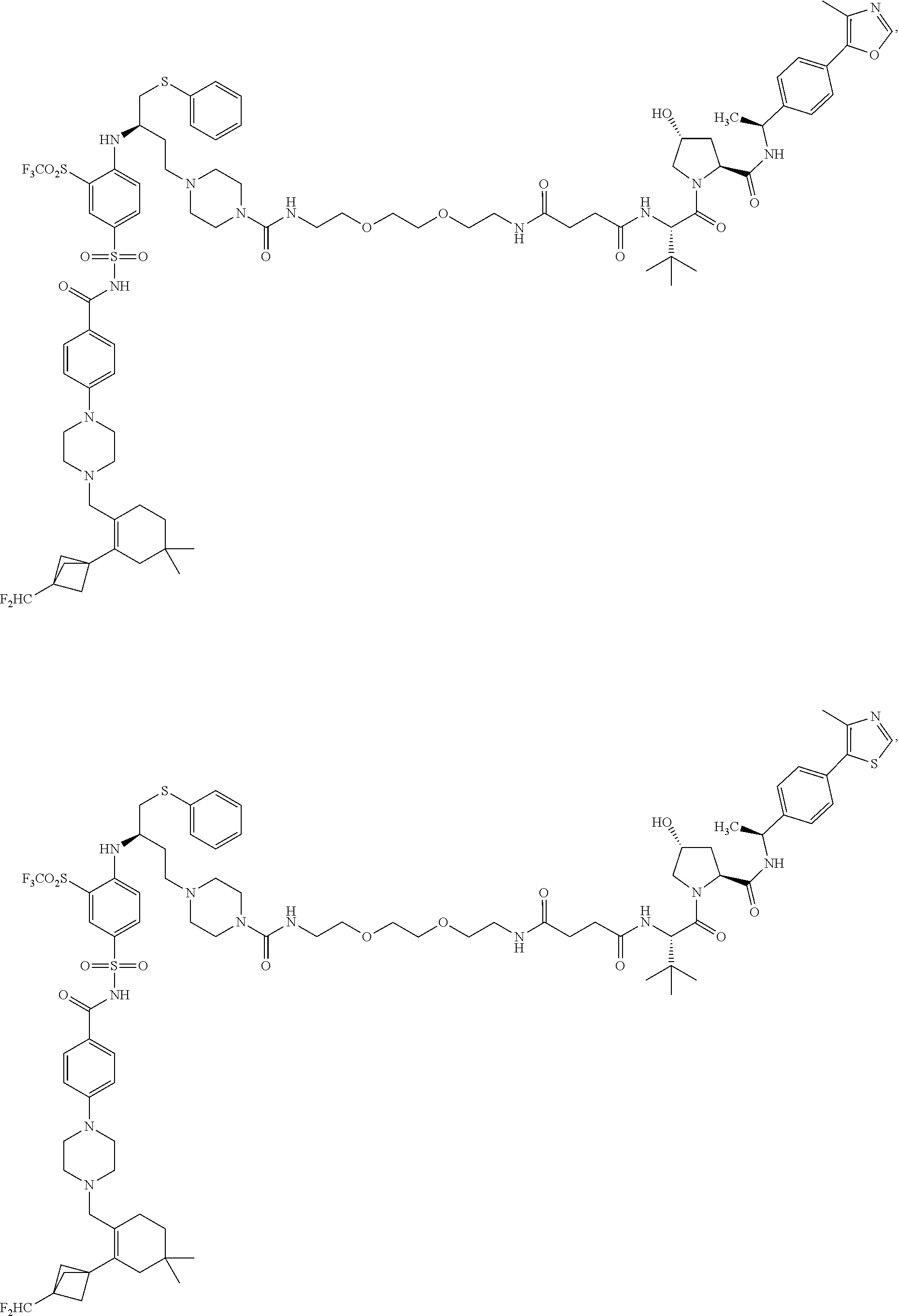 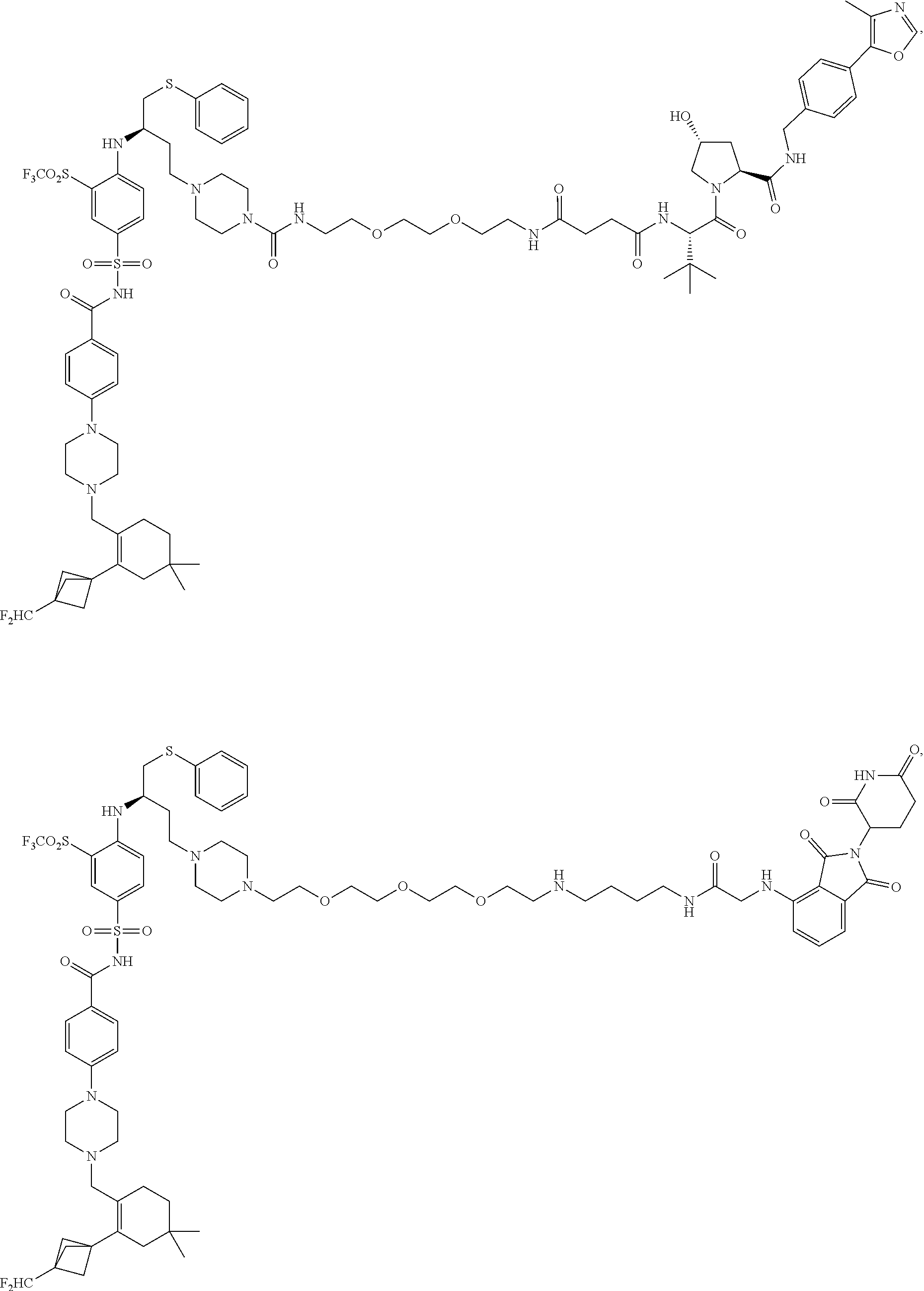 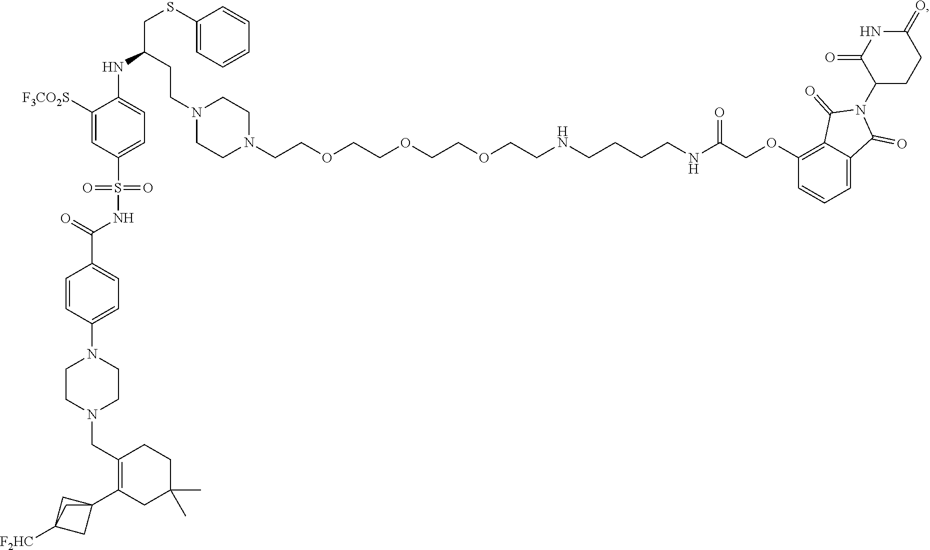 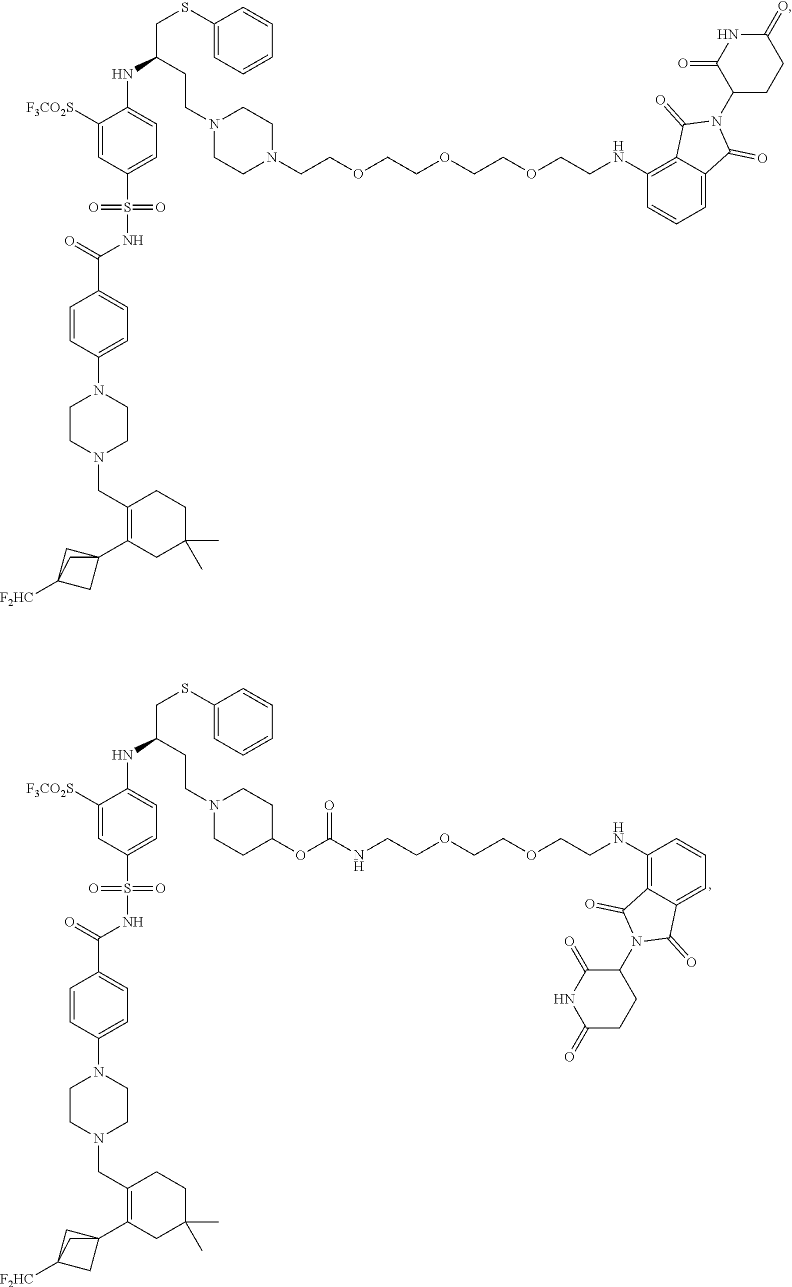 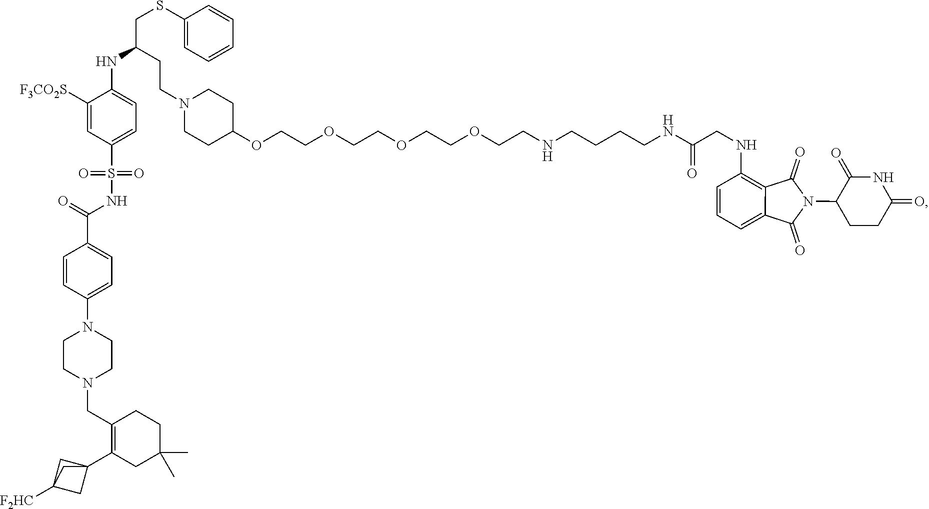 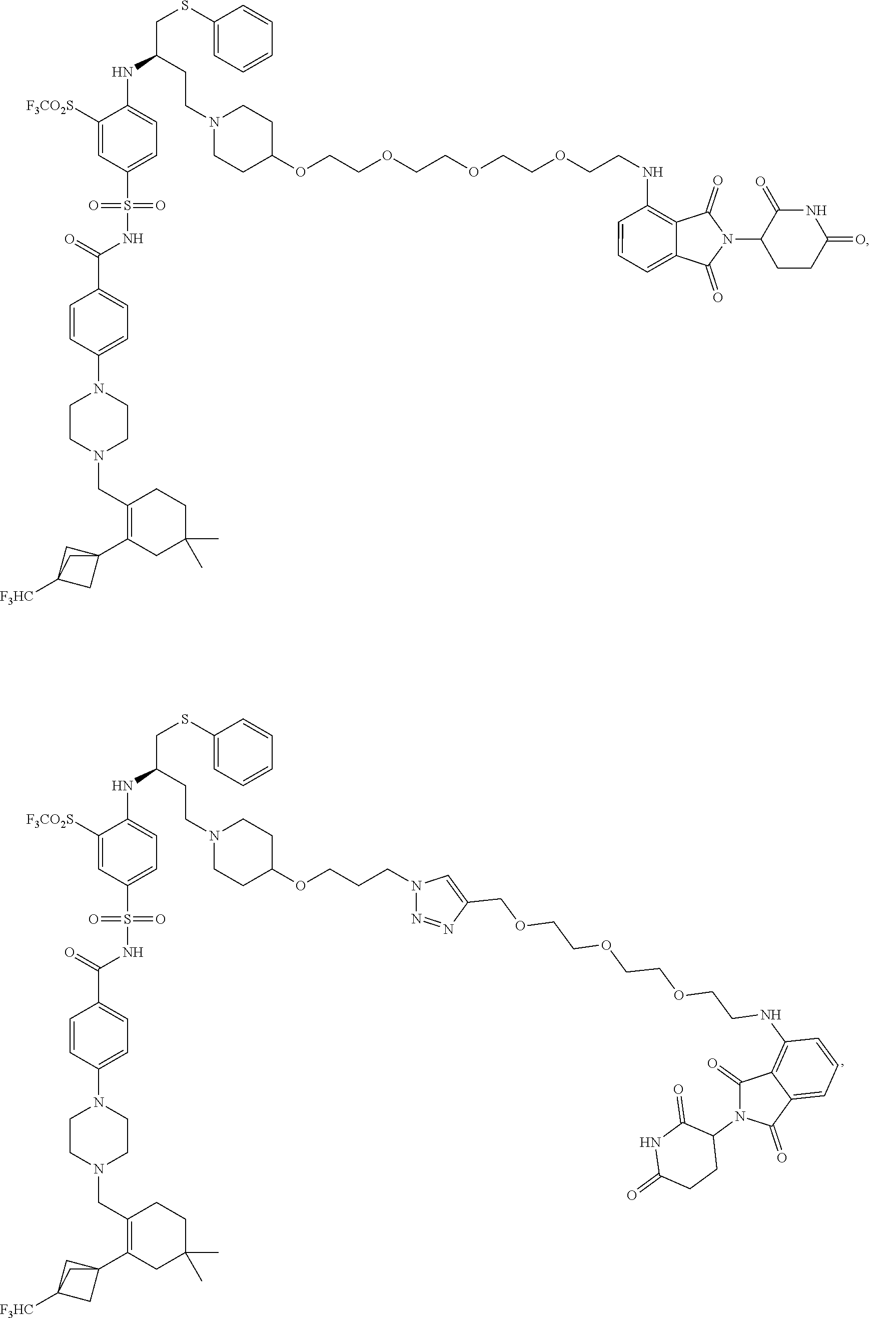 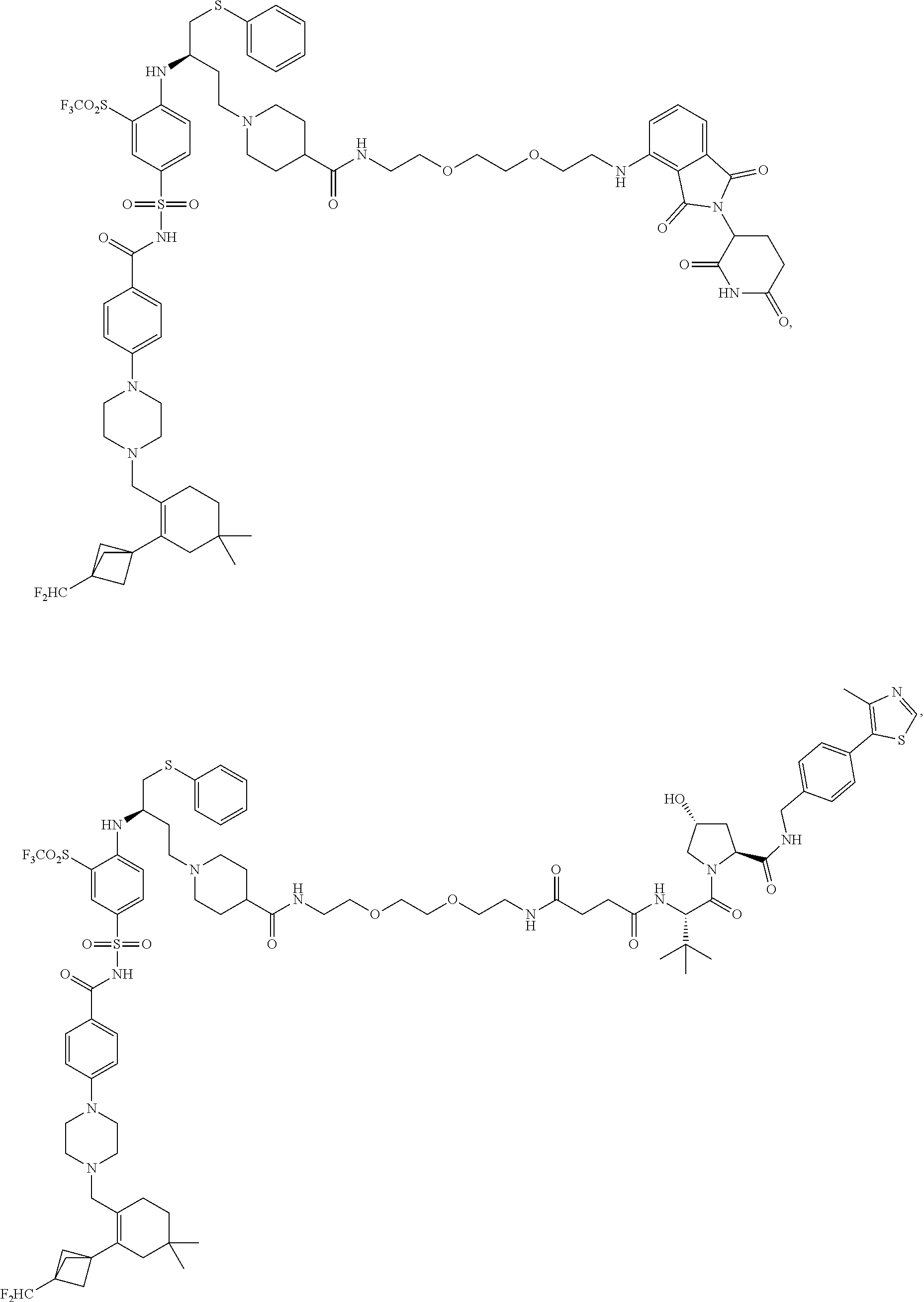 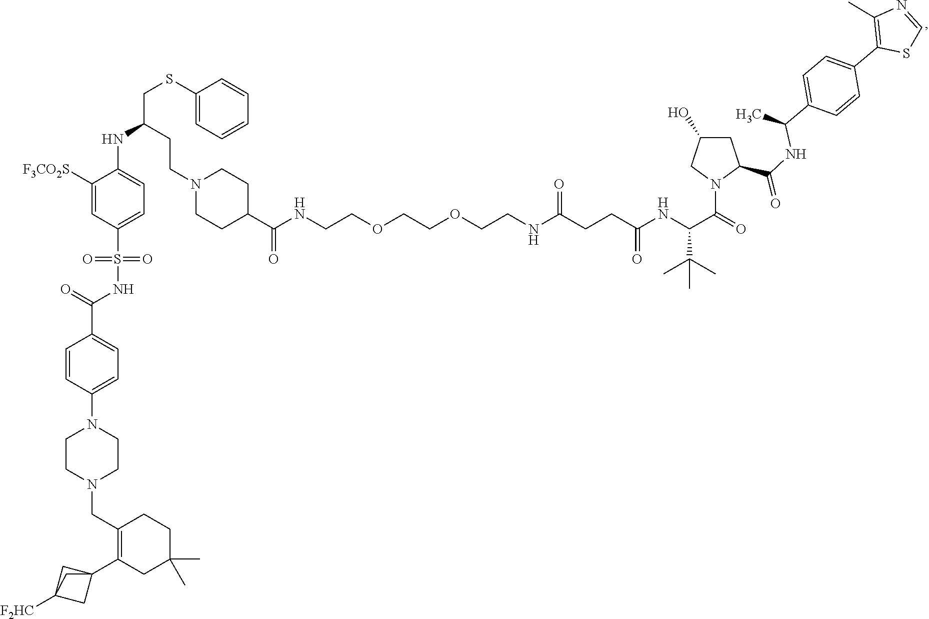 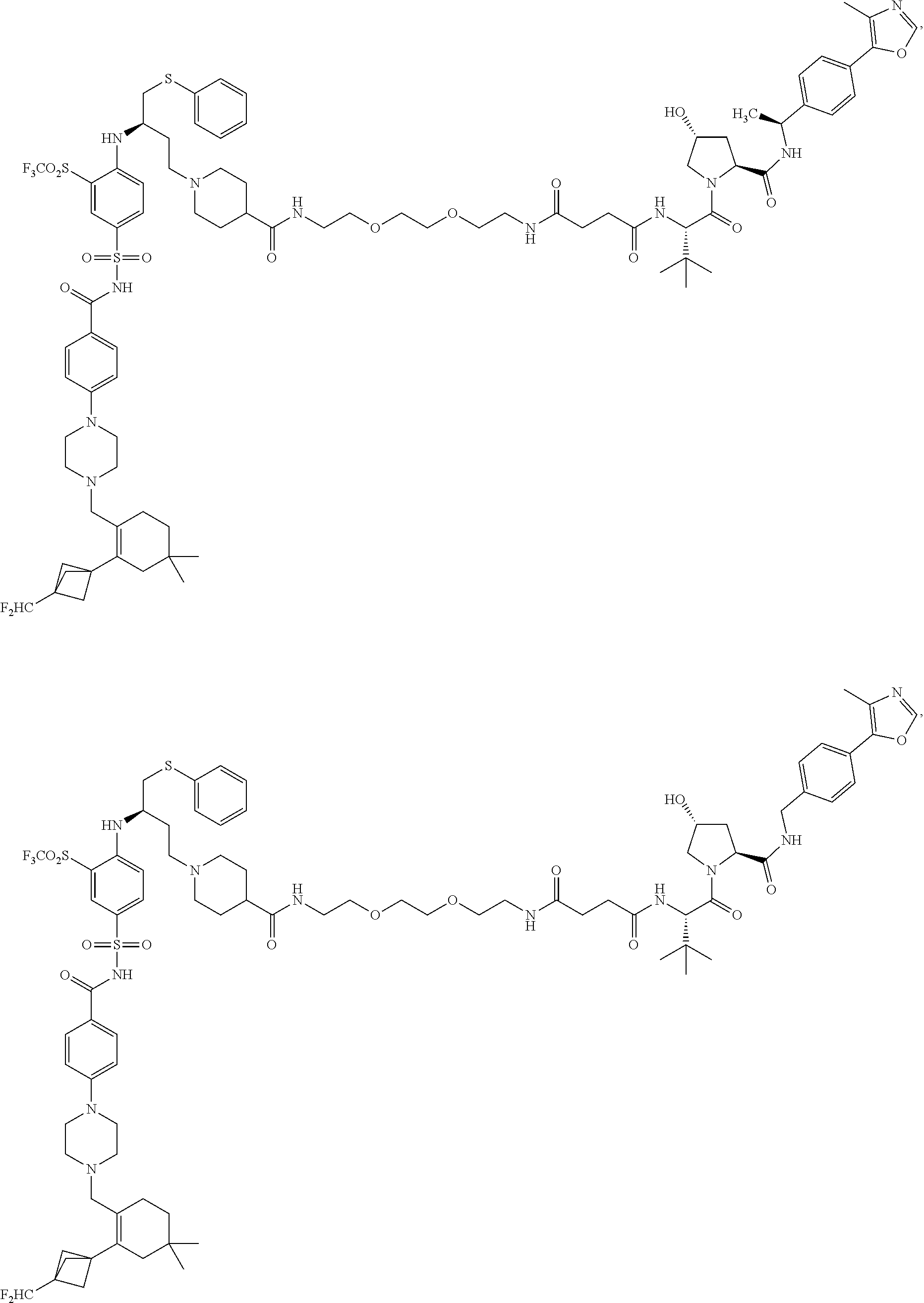 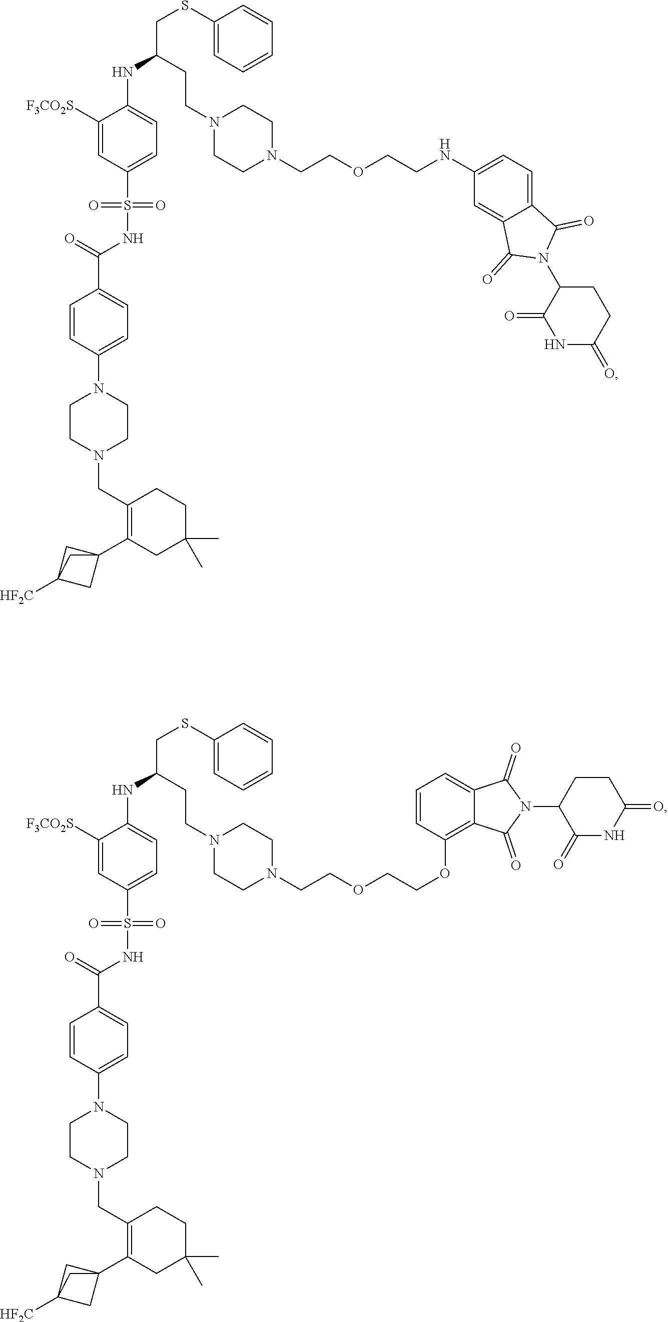 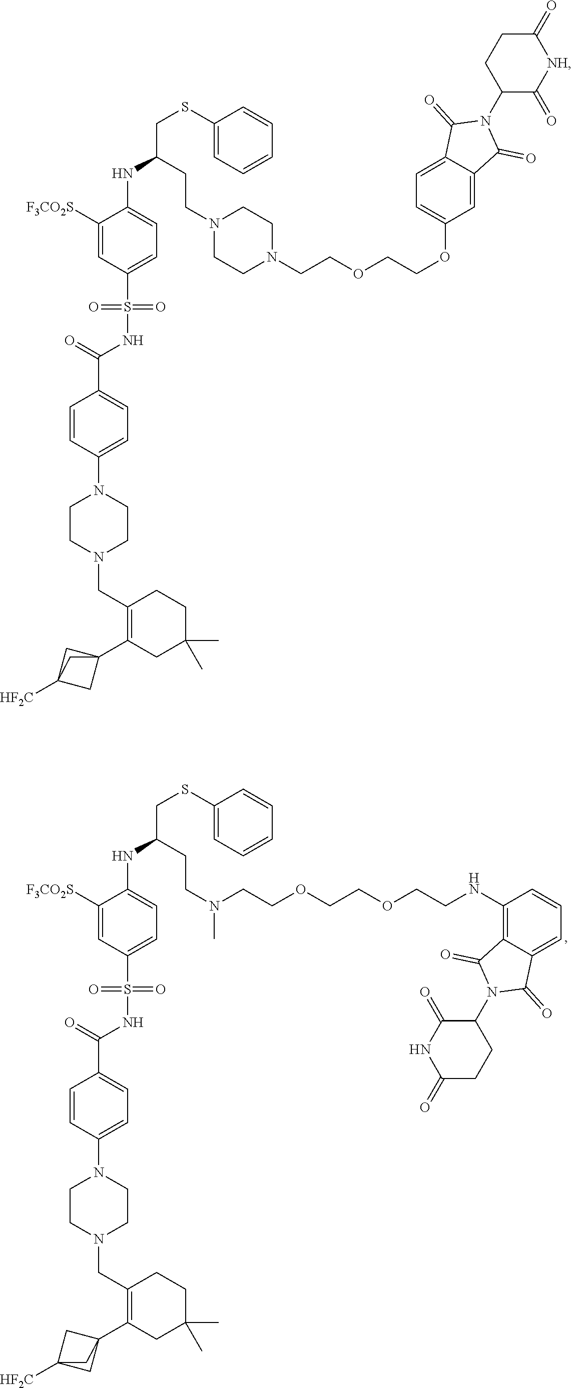 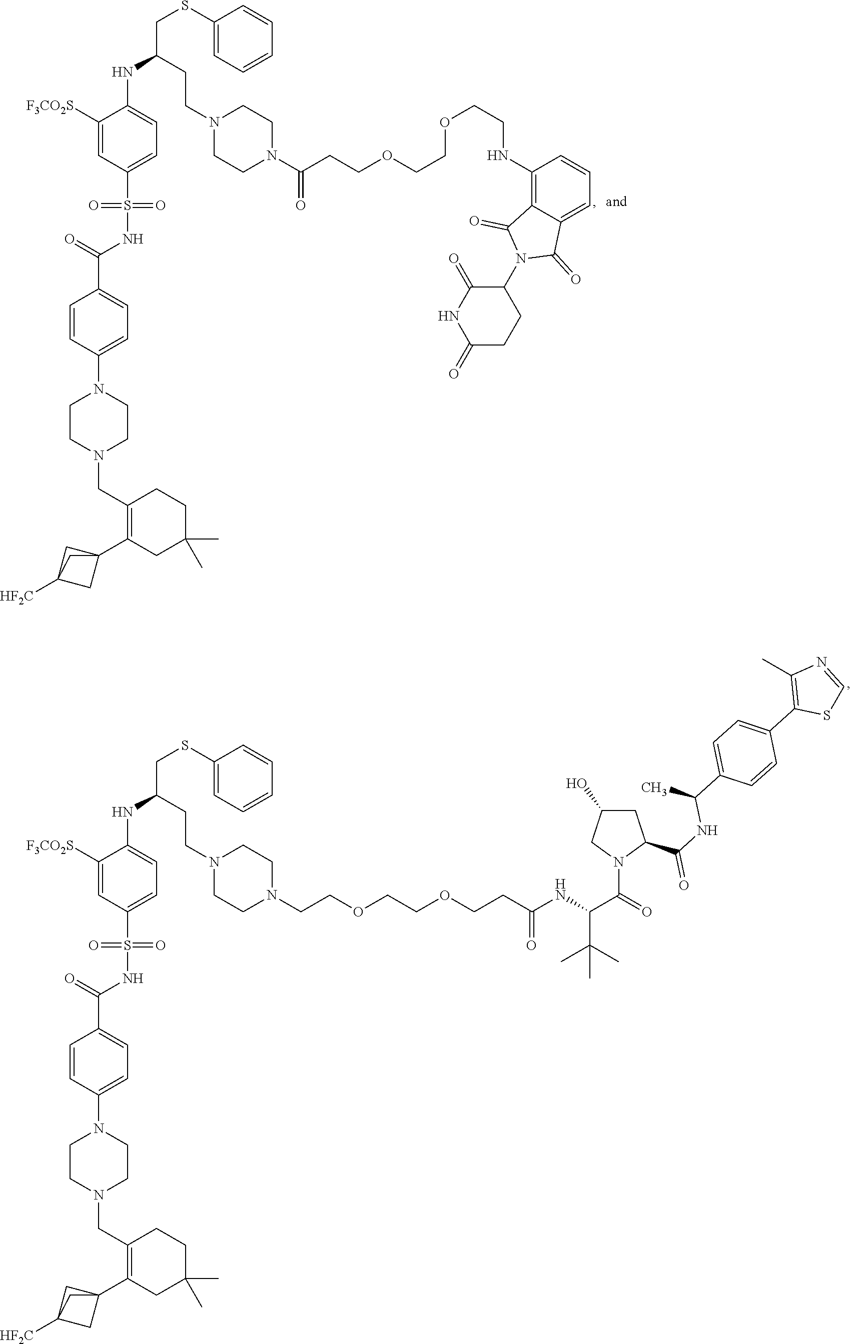
or a pharmaceutically acceptable salt of any of the foregoing.

65. A pharmaceutical composition comprising an effective amount of the compound of any one of any one of claims 1-64, or a pharmaceutically acceptable salt thereof, and a pharmaceutically acceptable carrier, diluent, excipient or combination thereof.

66. A method for treating a cancer or a tumor comprising administering an effective amount of a compound of any one of claims 1-64, or a pharmaceutically acceptable salt thereof, or a pharmaceutical composition of claim 65, to a subject having the cancer or the tumor, wherein the cancer or the tumor is selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

67. A method for inhibiting replication of a malignant growth or a tumor comprising contacting the growth or the tumor with an effective amount of a compound of any one of claims 1-64, or a pharmaceutically acceptable salt thereof, or a pharmaceutical composition of claim 65, wherein the malignant growth or tumor selected from an Ewings's tumor and a Wilm's tumor, or the malignant growth of tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma.

68. A method for treating a cancer comprising contacting a malignant growth or a tumor with an effective amount of a compound of any one of claims 1-64, or a pharmaceutically acceptable salt thereof, or a pharmaceutical composition of claim 65, wherein the malignant growth or tumor selected from an Ewings's tumor and a Wilm's tumor, or the malignant growth of tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma or an osteosarcoma.

69. A method for inhibiting the activity of a Bcl-2 protein and/or a Bcl-xL protein comprising providing an effective amount of a compound of any one of claims 1-64, or a pharmaceutically acceptable salt thereof, or a pharmaceutical composition of claim 65 to a cancer cell or a tumor, wherein the cancer cell or the tumor is from a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

70. A method for inhibiting the activity of a Bcl-2 protein and/or a Bcl-xL protein in a subject comprising providing an effective amount of a compound of any one of claims 1-64, or a pharmaceutically acceptable salt thereof, or a pharmaceutical composition of claim 65 to the subject having a cancer or a tumor, wherein the cancer or the tumor is selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

71. Use of an effective amount of a compound of any one of claims 1-64, or a pharmaceutically acceptable salt thereof, or a pharmaceutical composition of claim 65 in the manufacture of a medicament for treating a cancer or a tumor, wherein the cancer or the tumor is selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

72. Use of an effective amount of a compound of any one of claims 1-64, or a pharmaceutically acceptable salt thereof, or a pharmaceutical composition of claim 65 in the manufacture of a medicament for inhibiting replication of a malignant growth or a tumor, wherein the malignant growth or the tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

73. Use of an effective amount of a compound of any one of claims 1-64, or a pharmaceutically acceptable salt thereof, or a pharmaceutical composition of claim 65 in the manufacture of a medicament for treating a malignant growth or a tumor, wherein the malignant growth or the tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.
